(19) United States
(12) Patent Application Publication
(10) Pub. No.: US 2023/0414839 A1
(43) Pub. Date: Dec. 28, 2023

(54) COATED MEDICAL PRODUCT

(71) Applicant: HEMOTEQ AG, Würselen (DE)

(72) Inventors: Michael Hoffman, Roetgen (DE); Erika Hoffmann, Roetgen (DE); Günter Mathar, Stolberg (DE)

(21) Appl. No.: 18/036,664

(22) PCT Filed: Nov. 16, 2021
(86) PCT No.: PCT/EP2021/081898

§ 371 Date: May 12, 2023

(30) Foreign Application Priority Data
Nov. 16, 2020 (EP) EP 20 207 915.8
Sep. 16, 2021 (EP) EP 21 197 295.5

(51) Int. Cl. A61L 29/08; A61K 31/436; A61L 31/08; A61L 31/16; A61L 29/16

(52) U.S. Cl. CPC A61L 29/08; A61K 31/436; A61L 31/08; A61L 31/16; A61L 29/16; A61L 2300/63; A61L 2300/416; A61L 2300/802; A61L 2420/02; A61L 2300/606; A61L 2300/204; A61L 2300/608; A61L 2420/08

(57) ABSTRACT

The present invention relates to a suspension for coating of medical devices containing at least one tri-O-acylglycerol, at least one limus active agent in the form of microcrystals and at least one solvent in which the at least one tri-O-acylglycerol dissolves and in which the microcrystals of the at least one limus active agent do not dissolve. Furthermore, the present invention relates to a pmethod for preparing said suspension, a p method for coating a medical device with said suspension, and medical devices coated with at least one tri-O-acylglycerol and at least one microcrystalline limus active agent.

### FIELD OF INVENTION

The present invention relates to a suspension for coating of medical devices comprising at least one tri-O-acylglycerol, at least one microcrystalline limus active agent, and at least one solvent or solvent mixture, in which the at least one tri-O-acylglycerol dissolves and in which the microcrystals of the at least one limus active agent do not dissolve when the at least one tri-O-acylglycerol is present. The present invention further relates to methods for preparing said suspension, methods for coating medical devices, and medical devices coated with at least one tri-O-acylglycerol and at least one microcrystalline limus active agent.

### BACKGROUND OF THE INVENTION

Medical devices are used to take over functions that are missing in the body, to support the body's own functions or to transfer active substances locally with their help. Depending on the area of application, medical devices have either short- or long-term contact with an organism. The contact time can range from a few seconds to decades. If the use of a medical device becomes necessary, it is necessary to control the inevitable inflammatory processes that occur during wound healing in order to prevent overreactions of the immune system in the healing process.

When treating vasoconstrictions (stenoses) with mechanical or thermal procedures, such as implantation of vascular stents or balloon angioplasty, restenosis occurs as a frequent complication a few weeks after treatment. To prevent restenosis, stents and catheter balloons have been coated with active agents, especially antirestenotic agents. In the past, limus active agents such as rapamycin (sirolimus) or taxanes such as paclitaxel have proven to be successful active agents. Limus active agents bind reversibly to FKBP12 and suppress cell division, whereas taxanes such as paclitaxel bind irreversibly to mictrotubules and also suppress cell division.

“Drug-eluting stents” (DES) are known in the prior art in which, in addition to vessel dilation and the associated injury to the vessel wall, healing at the affected site is to be controlled with the aid of suitable active agents.

Medical devices that do not remain permanently in the body, such as biodegradable stents, are also known in the art. The biodegradable stents may additionally have a drug coating to provide the benefits of a long-term medical device. However, this approach is still under development.

In addition to stents, drug-eluting catheters, in particular balloon catheters, are also known in the prior art, which have the advantage that they only come into contact with the organism for a short period of time.

However, not only stents and catheters can be coated with active agents, although the requirements for coating of other medical devices naturally differ depending on the area of application.

The requirements for active agent-releasing coatings of catheters are particularly high, since a long-term, well-dosed and yet as quantitative as possible application of active agents beyond the very short retention time of the medical device represents a special challenge, especially in the case of the medical devices, which are used for a very short period of time, especially in the vascular area, whereby it must be ensured that, on the one hand, the active agent is not flushed away prematurely on its way to the target site or, for example, crumbles away during expansion and only an undefined or insufficient amount of active agent reaches the vessel wall. On the other hand, in the case of a coronary “drug-coated balloon” (DCB) as a medical device used for a short period of time, the very limited contact time of maximum 90 seconds must also be sufficient for the active agent to be transferred in the intended dosage from the balloon catheter to or into the vessel wall. The peripheral vascular system, e.g. in the leg artery, allows longer contact times of around 120 seconds and more, depending on which vessel is being treated, with the upper limit of contact time in peripheral vessels being a maximum of 5 minutes in the superficial femoral artery (AFS).

In the prior art, some active agent coatings of catheter balloons are known that circumvent these problems by accelerating the drug release or increasing the stability of the coating by using additional excipients in the coating (see WO2010/121840A2, WO2013/007653A1, WO2012/146681A1).

Particularly in the case of catheter balloons coated with limus compounds, another reason for the low efficacy, in addition to the low drug transfer, is the short retention time of the limus compound in the vessel wall.

It is known from the prior art that limus active agent crystals exhibit slower dissolution behavior than amorphous limus particles. Thus, higher drug concentrations were observed in the vessel wall one month after treatment with catheter balloons coated with a crystalline limus active agent compared with treatment with catheter balloons with an amorphous active agent coating (Clever et al., Circ. Cardiovasc. Interv. 2016, 9, 1-11; e003543).

Coatings with crystalline limus active agents are therefore desirable to ensure a prolonged retention time of the limus active agent in the vessel wall. However, the use of crystalline balloon coatings carries the risk of embolism (WO2011/147408A2), so amorphous coatings are still preferred for catheter balloons in the prior art.

The application of limus crystals by means of balloon dilation to the tissue to be treated has the advantage that the crystals act as drug depots and release the drug with a delay, whereas with amorphous drugs there is an immediate release after dilation. However, it has been shown that direct coating with limus crystals or coating with a pure crystal-containing suspension of limus active agent has the particular disadvantage that the limus crystals do not adhere sufficiently to the medical device surface. Another problem is that suspensions of particles larger than 1-5 μm tend to sediment rapidly, which subsequently makes uniform coating with microcrystalline limus active agent from suspensions much more difficult.

Active agent coatings with crystalline limus active agent for catheter balloons are known from the prior art, which attempt to circumvent these problems. However, the prior art methods for coating catheter balloons with crystalline limus compounds are costly and complicated.

The international patent application WO2013/022458A1 discloses a process for converting amorphous everolimus into its crystalline form by aging a supersaturated solution (suspension) over several days. The lower solubility of the crystalline compared to the amorphous form is exploited.

In the prior art, mainly limus crystal suspensions are proposed in which the size of the limus crystals is in the nanoscale of less than 1 μm. However, crystals of this size have the disadvantage that they cannot sufficiently ensure the desired extended retention time of the limus active agent in the vessel wall.

The international patent application WO2015/039969A1 discloses a coating process of balloon catheters with crystalline limus active agents. The crystalline limus active agents were either prepared beforehand and applied as a suspension to the balloon catheter, or crystallization was induced on the balloon by seed crystals. However, crystallization on the surface has the disadvantage that, in addition to crystallization, precipitation processes occur that lead to amorphous particles or agglomerates with a size of 100-300 μm, which cannot be dispersed by ultrasonic treatment. Such agglomerates with a size of more than 100 μm bear the risk of causing vessel occlusions distal to the dilatation site during dilatation and can thus pose a considerable risk to the patient. It is therefore particularly desirable to keep the number of such amorphous particles or agglomerates as low as possible.

Sufficiently strong, reproducible coatings of catheter balloons with limus crystals that exhibit adequate drug transfer upon dilation, could not be obtained by coating a catheter balloon with a solution consisting of a solvent and a limus active agent as well as by sputtering limus crystals onto the balloon surface followed by optional adhesion enhancement by “solvent bonding” and by crystallization of a dissolved limus active agent from a solution of the limus active agent in a solvent and a non-solvent.

The objective of the present invention is to provide coating formulations and coated medical devices, wherein the coating is flexible, adheres very well to the medical device surface, has an optimal size distribution of the active agent particles, and delivers the active agent as quantitatively as possible even within a very short residence time in the body, whereby the active agent can then also diffuse from the vessel wall into the cells over a much longer period of time.

With other words, the objective of the present invention is to provide compositions for coatings of short- and long-term medical devices, which adhere to the surface of the medical device as a coating in a stable yet flexible manner and, on the other hand, ensure the most complete and controlled possible transfer of active agent to the vessel wall or tissue in order to optimally support the healing process.

In particular, the objective of the present invention is to provide a coating of catheter balloons with crystalline limus compounds, wherein the coating adheres to the surface of the catheter balloons in a stable yet flexible manner and, on the other hand, ensures a complete as well as controlled drug transfer to the vessel wall or tissue during dilatation in order to optimally support the healing process.

This objective is solved by the technical teaching of the independent claims of the present invention. Further advantageous embodiments of the invention result from the dependent claims, the description, the figures as well as the examples.

### BRIEF DESCRIPTION

Surprisingly, it has been found that a suspension for coating of medical devices, in particular of catheter balloons, balloon catheters, stents and cannulas, containing a) at least one tri-O-acylglycerol selected from trioctanoylglycerol, trinonanoylglycerol, tridecanoylglycerol, and triundecanoylglycerol, and b) at least one limus active agent in the form of microcrystals, and c) a solvent or a solvent mixture in which the at least one tri-O-acylglycerol dissolves and in which the microcrystals of the at least one limus active agent do not dissolve or in which the microcrystals of the at least one limus active agent do not dissolve when the at least one tri-O-acylglycerol is present, solves the above objective.

It has now been found that a particularly advantageous crystal suspension of a microcrystalline limus active agent for coating of medical devices, in which the microcrystals of the limus active agent do not dissolve, can be provided if at least one tri-O-acylglycerol selected from trioctanoylglycerol, trinonanoylglycerol, tridecanoylglycerol, and triundecanoylglycerol is present in dissolved form in said suspension.

With the tri-O-acylglycerols according to the invention, it was surprisingly possible to prepare such stable suspensions of microcrystalline limus active agents that the microcrystals of the limus active agents are kept “in abeyance” and thus did not sediment. This was particularly surprising since suspensions of crystals in the micrometer range, i.e. crystals larger than 1-5 μm tend to sediment. The crystal suspension according to the invention is thus particularly advantageous for producing a uniform coating of microcrystals of a limus active agent on medical devices.

Another particular advantage of using at least one tri-O-acylglycerol selected from trioctanoylglycerol, trinonanoylglycerol, tridecanoylglycerol, and triundecanoylglycerol, is that the tri-O-acylglycerols according to the invention are capable of holding the microcrystals of the limus active agent on a medical device surface like “flexible adhesives”. The crystal suspension according to the invention is thus particularly advantageous for producing a uniform coating of microcrystals of a limus active agent on medical devices, in which the microcrystals of the limus active agent also adhere sufficiently to the medical device surface.

With further prior art tri-O-acylglycerols not according to the invention, the preparation of crystal suspensions according to the present invention was not possible. It could be shown that tri-O-acylglycerols not according to the invention cause or promote dissolution of the microcrystals of the limus active agent in the suspension. In addition, sedimentation of the microcrystals of the limus active agent occurred with tri-O-acylglycerols not according to the invention.

In addition, with further prior art tri-O-acylglycerols not according to the invention no uniform coatings of microcrystals of a limus active agent could be produced on medical devices, and in particular a lack of adhesion of the microcrystals of the limus active agent to the medical device surface has been observed. It could be shown that tri-O-acylglycerols not according to the invention cannot sufficiently “stick” and hold the microcrystals of the limus active agent on a medical device surface.

Thus, only with the tri-O-acylglycerols selected from trioctanoylglycerol, trinonanoylglycerol, tridecanoylglycerol, and triundecanoylglycerol or also with mixtures of these tri-O-acylglycerols, a crystal suspension of a microcrystalline limus active agent according to the invention could be prepared, in which the microcrystals of the limus active agent remain intact. A further particular advantage is that the microcrystals of the at least one limus active agent are floating in the suspension and are therefore uniformly distributed in the suspension, so that the limus active agent can be applied not only in the form of microcrystals but also uniformly on the medical device surface.

Medical devices that have been coated with a suspension according to the invention exhibit a coating of at least one tri-O-acylglycerol selected from trioctanoylglycerol, trinonanoylglycerol, tridecanoylglycerol, and triundecanoylglycerol, and microcrystals of the at least one limus active agent on the medical device surface. This coating is primarily characterized by a very good flexibility and an excellent adhesion to the medical device surface. Furthermore, this coating offers the advantageous property that even with a very short residence time in the body, the microcrystals of the at least one limus active agent can be quantitatively released, which can then diffuse from the vessel wall into the cells over a much longer period of time, in contrast to amorphous limus active agent particles.

A coating according to the invention can be provided on any medical device, preferred herein are catheter balloons, balloon catheters, stents and cannulas, particularly preferred are catheter balloons. The limus active agent amount and the limus active agent delivery rate or elution rate may vary according to the required specifications at the operation site, while the at least one tri-O-acylglycerol selected from trioctanoylglycerol, trinonanoylglycerol, tridecanoylglycerol, and triundecanoylglycerol, on the one hand, supports optimal transfer of the microcrystalline limus active agent into the tissue and, at the same time, ensures high flexibility and stability of the coating, thus guaranteeing that the microcrystalline limus active agent actually reaches the surrounding tissue in optimal concentration without losses.

The very good flexibility and adhesion of the coating according to the invention is particularly important for medical devices that have to undergo changes in shape, e.g. stents and catheter balloons. For example, inflation, deflation, folding and crimping require special stability requirements for a coating that is also exposed to friction and body fluids as well as flows during implantation. Setting the desired elution rate of the microcrystalline limus active agent and the most optimal transfer amount of microcrystalline limus active agent into the tissue are also solved with a catheter balloon coating (DCB) according to the invention. Further requirements to be considered, which the coating according to the invention fulfills without prejudice, relate to sterilization of the medical devices, shelf life, minimum shelf life, temperature resistance and the like.

Thus, the inventive coating on medical devices solves the important tasks that are demanded for a medical device used in the body in the long term and also in the short term.

### DETAILED DESCRIPTION

The present invention relates to a suspension for coating of medical devices, preferably catheter balloons, balloon catheters, stents and cannulas, the suspension containing:
a) at least one tri-O-acylglycerol selected from the group consisting of trioctanoylglycerol, trinonanoylglycerol, tridecanoylglycerol, and triundecanoylglycerol, and
b) at least one limus active agent in the form of microcrystals, and
c) a solvent or a solvent mixture in which the at least one tri-O-acylglycerol dissolves and in which the microcrystals of the at least one limus active agent do not dissolve.

Essential to the invention is the use of microcrystalline limus active agent and the presence of a suspension of the microcrystalline limus active agent wherein the suspension must contain at least one tri-O-acylglycerol selected from the group consisting of trioctanoylglycerol, trinonanoylglycerol, tridecanoylglycerol, and triundecanoylglycerol.

The term “coating formulation” or “active agent-containing composition”, as used herein, refers to a mixture of at least one limus active agent and a solvent or a solvent mixture and at least one tri-O-acylglycerol selected from the group consisting of trioctanoylglycerol, trinonanoylglycerol, tridecanoylglycerol, and triundecanoylglycerol, i.e., a solution, dispersion, suspension or emulsion. The term “formulation” is intended to indicate that it is a liquid mixture (suspension, emulsion, dispersion, solution). The term “coating formulation”, as used herein, thus represents the generic term for the terms “solution” or “coating solution”, “dispersion” or “coating dispersion”, “suspension” or “coating suspension” and “emulsion” or “coating emulsion”.

The term “solution” or “coating solution”, as used herein, generally refers to a homogeneous mixture consisting of two or more chemically pure substances. Solutions are not externally recognizable as such, since by definition they form only one phase and the solutes are uniformly distributed in the solvent.

The term “dispersion” or “coating dispersion”, as used herein, generally refers to a heterogeneous mixture of at least two substances that do not or hardly dissolve in each other or chemically combine with each other. In this case, one or more substances are finely dispersed as a disperse phase in another continuous substance, the so-called dispersion medium.

The term “emulsion” or “coating emulsion”, as used herein, generally refers to finely distributed mixture of two normally immiscible liquids without visible segregation. One liquid forms small droplets dispersed in the other liquid. The emulsion is a particular form of a dispersion.

The term “suspension” or “coating suspension”, as used herein, generally refers to a heterogeneous mixture of substances consisting of a finely distributed solid in a liquid. Thus, by definition, a “suspension” is not a homogeneous mixture and thus not a solution. The suspension is a specific form of a dispersion.

A “suspension” containing at least one limus active agent in the form of microcrystals is also referred to herein as a “crystal suspension”. According to the present invention, the finely distributed solid of the suspension herein is at least one microcrystalline limus active agent or microcrystals of at least one limus active agent. The liquid of the suspension herein is a solvent or a solvent mixture, wherein at least one tri-O-acylglycerol selected from the group consisting of trioctanoylglycerol, trinonanoylglycerol, tridecanoylglycerol, and triundecanoylglycerol is present in the dissolved form in the solvent or the solvent mixture.

Thus, a “suspension” according to the present invention relates to a heterogeneous mixture of substances of a liquid containing at least one tri-O-acylglycerol selected from the group consisting of trioctanoylglycerol, trinonanoylglycerol, tridecanoylglycerol, and triundecanoylglycerol, and solids finely distributed in this liquid, namely the microcrystals of the at least one limus active agent. Thus, according to the invention, the microcrystalline limus active agent is suspended in a liquid containing at least one dissolved tri-O-acylglycerol selected from the group consisting of trioctanoylglycerol, trinonanoylglycerol, tridecanoylglycerol, and triundecanoylglycerol.

The suspension according to the present invention is characterized in that neither sedimentation nor dissolution of the microcrystals of the at least one limus active agent occurs in the suspension. The suspension according to the present invention is also referred to herein as a “stable suspension”.

The suspension according to the invention may consist of the solvent or the solvent mixture and the microcrystalline limus active agent and the at least one dissolved tri-O-acylglycerol selected from the group consisting of trioctanoylglycerol, trinonanoylglycerol, tridecanoylglycerol, and triundecanoylglycerol. However, the suspension may contain up to 5.0% by weight of other additives based on the limus active agent, i.e. for 95 g of limus active agent, up to 5 g of additives may be contained in the suspension. Only in the case of antioxidants as additives, the amount of antioxidants may be up to 15% by weight, but the amount of antioxidants and all other additives may still not exceed 15% by weight, i.e. for 85 g of limus active ingredient, up to 15 g of antioxidants may be contained in the suspension. Thus, if 15% by weight of antioxidants are present in the suspension, then no other additives can be present. If, on the other hand, 10% by weight of antioxidants are present in the suspension, then other additives can also be present up to a maximum of 5.0% by weight.

Thus, the present invention also relates to a suspension for coating of medical devices, preferably selected from a catheter balloon, a catheter, a stent or a cannula, the suspension consisting of:
a) at least one tri-O-acylglycerol selected from the group consisting of trioctanoylglycerol, trinonanoylglycerol, tridecanoylglycerol, and triundecanoylglycerol, and
b) at least one limus active agent in the form of microcrystals, and
c) a solvent or a solvent mixture in which the at least one tri-O-acylglycerol dissolves and in which the microcrystals of the at least one limus active agent do not dissolve, und
d) up to 5.0% by weight, based on the limus active agent, of additives or up to 15.0% by weight, based on the limus active agent, of antioxidants as additives and up to 5.0% by weight, based on the limus active agent, of additives which are not antioxidants, the total amount of additives not exceeding 15.0% by weight, based on the limus active agent.

Suitable additives are the substances mentioned below, preferably antioxidants, polyvinylpyrrolidone (PVP) and flocculation inhibitors.

The antioxidants and preferably BHT are preferably present in the suspension in an amount of up to 12.0% by weight based on the limus active agent, more preferably up to 10.0% by weight based on the limus active agent, more preferably up to 9.0% by weight based on the limus active agent, more preferably up to 8.0% by weight based on the limus active agent and more preferably up to 7.0% by weight based on the limus active agent. Other additives, such as PVP or flocculation inhibitors, which do not belong to the antioxidants, can preferably be present in an amount of up to 4.0% by weight based on the limus active agent, preferably up to 3.0% by weight based on the limus active agent, further preferably up to 2.5% by weight based on the limus active agent, further preferably up to 2.0% by weight based on the limus active agent, further preferably up to 1.5% by weight based on the limus active agent and further preferably up to 1.0% by weight based on the limus active agent.

The term “tri-O-acylglycerol,” or short form “triacylglycerol”, as used herein, refers to a chemical compound of glycerol (glycerin) esterified with three fatty acids, i.e., triple esterified glycerols (glycerins). Triglyceride or glycerol triester are synonymous terms of tri-O-acylglycerol, the term tri-O-acylglycerol is recommended by IUPAC.

Tri-O-acylglycerols have the following general formula (1):

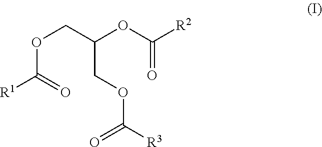

wherein R¹, R² and R³ represent alkyl or alkenyl residues. The structure of tri-O-acylglycerols is diverse, since R¹, R² and R³ allow many different fatty acids and thus a high number of possible combinations. All are non-polar, i.e. lipophilic. In the case of tri-O-acylglycerols, a further distinction can be made between medium-chain and long-chain tri-O-acylglycerols. Medium-chain tri-O-acylglycerols have fatty acids with an average length of 6 to 12 carbon atoms, and long-chain tri-O-acylglycerols have fatty acids with a length of 14 to 24 carbon atoms. Thereby, two types of tri-O-acylglycerols can arise: simple and mixed tri-O-acylglycerols. In simple tri-O-acylglycerols, the fatty acid residues R¹, R² and R³ are identical; in mixed ones, at least one of the fatty acid residues R¹, R² and R³ is different from the other two. Examples of medium-length fatty acids are caproic acid (hexanoic acid), enanthic acid (heptanoic acid), caprylic acid (octanoic acid), perlargonic acid (nonanoic acid), capric acid (decanoic acid), undecanoic acid and lauric acid (dodecanoic acid).

Unexpectedly, it could be shown that the chemical, physical and biological properties of tri-O-acylglycerols fully esterified with the medium-length fatty acids caprylic acid (octanoic acid), capric acid (decanoic acid), perlargonic acid (nonanoic acid) or undecanoic acid enable a uniform and sufficiently adhesive coating of medical devices with microcrystalline limus active agents in the first place. Moreover, only the glycerols fully esterified with caprylic acid (octanoic acid), capric acid (decanoic acid), perlargonic acid (nonanoic acid) or undecanoic acid it was possible to prepare a crystal suspension according to the invention.

Thus, the herein preferred tri-O-acylglycerols have the following general formula (1):

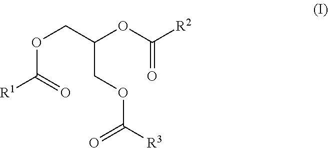

wherein R¹, R², and R³ are independently of each other selected from —CH₂(CH₂)₅CH₃, —CH₂(CH₂)₆CH₃, —CH₂(CH₂)₇CH₃ and —CH₂(CH₂)₈CH₃.

It could be shown that with mixed tri-O-acylglycerols in which R¹, R² and R³ are independently selected from —CH₂(CH₂)₅CH₃, —CH₂(CH₂)₆CH₃, —CH₂(CH₂)₇CH₃ and —CH₂(CH₂)₈CH₃, and wherein not all three R¹, R² and R³ are identical, stable crystal suspensions of microcrystalline limus active agents can also be prepared. However, these mixed tri-O-acylglycerols are more costly to prepare and are not cost effective, so they are not preferred herein.

Therefore, according to the present invention, all three residues R¹, R² and R³ are identical, i.e. R¹, R² and R³ are —CH₂(CH₂)₅CH₃, or R¹, R² and R³ are —CH₂(CH₂)₆CH₃, or R¹, R² and R³ are —CH₂(CH₂)₇CH₃, or R¹, R² and R³ are —CH₂(CH₂)₈CH₃.

In attempts to prepare crystal suspensions containing tri-O-acylglycerols, which are completely esterified with other medium-length fatty acids not according to the invention as mentioned above, in particular the shorter medium-length fatty acids such as caproic acid (tricaproic), but also the shorter monocarboxylic acids such as acetic acid (triacetin), it was not possible to prepare crystal suspensions according to the invention, because in the presence of these tri-O-acylglycerols not according to the invention, dissolution of the microcrystals of the limus active agent in the suspension occurred or was promoted. Dissolution of the microcrystals of the limus active agent in the suspension also occurred with glycerols only partially esterified with medium-length fatty acids, such as the mono-O-acylglycerols or di-O-acylglycerols. In addition, a sedimentation of the microcrystals of the limus active agent occurred with the tri-O-acylglycerols not according to the invention.

In complete absence of tri-O-acylglycerols in the suspension, sedimentation of the microcrystals of the at least one limus active agent occurred rapidly. Preparation of a stable suspension was not possible.

The presence of at least one dissolved tri-O-acylglycerol selected from the group consisting of trioctanoylglycerol, trinonanoylglycerol, tridecanoylglycerol, and triundecanoylglycerol in the suspension is thus essential for the crystal suspension according to the present invention.

Thus, it could be shown that the glycerol fully esterified with three octanoic acid molecules, the glycerol fully esterified with three decanoic acid molecules, the glycerol fully esterified with three nonanoic acid molecules or the glycerol fully esterified with three undecanoic acid, i.e. at least one tri-O-acylglycerol selected from the group consisting of trioctanoylglycerol, trinonanoylglycerol, tridecanoylglycerol, and triundecanoylglycerol, does not partially dissolve or does not dissolve microcrystalline limus active agents in a suspension.

Thus, with the tri-O-acylglycerols according to the invention a crystal suspension can be provided as a coating formulation in which the microcrystals of the limus active agent remain intact, suspended and uniformly distributed in the suspension, and no sedimentation of the microcrystals of the limus active agent or agglomeration of particles occurs, so that the limus active agent can be uniformly applied in microcrystalline form to a medical device surface.

Another particular advantage of using at least one tri-O-acylglycerol selected from the group consisting of trioctanoylglycerol, trinonanoylglycerol, tridecanoylglycerol, and triundecanoylglycerol is that the tri-O-acylglycerols according to the invention are capable of holding the microcrystals of the limus active agent on a medical device surface like a “flexible adhesive”, so that sufficient adhesion of the microcrystals of the limus active agent to a medical device surface can be provided.

The tri-O-acylglycerols according to the invention selected from the group consisting of trioctanoylglycerol, trinonanoylglycerol, tridecanoylglycerol, and triundecanoylglycerol, or a mixture of said tri-O-acylglycerols have the advantage that their melting points make them safe for use in the body. It has also been found that a melting point of below 37° C. is essential to ensure adequate adhesion of the microcrystals of the limus active agent to a medical device surface. Only the tri-O-acylglycerols according to the invention selected from the group consisting of trioctanoylglycerol, trinonanoylglycerol, tridecanoylglycerol, and triundecanoylglycerol, or mixtures of said tri-O-acylglycerols can hold microcrystalline limus active agents like a “glue”, thus guaranteeing optimum flexibility and loss-free transport to the operation site. Even the next higher homolog, tridodecanoylglycerol, has the disadvantage that it melts at 45-46° C.

With tri-O-acylglycerols not according to the invention, which are fully esterified with medium-length fatty acids or long-chain fatty acids not according to the invention as mentioned above, such as lauric acid, myristic acid or palmitic acid, it was not possible to provide a crystal suspension according to the invention, in which the microcrystals of the limus active agent float in the suspension and are uniformly distributed in the suspension. When using tri-O-acylglycerols not according to the invention, such as tridodecanoylglycerol, uniform coating with limus active agent in microcrystalline form on medical device surfaces was not possible. Coated catheter balloons with a coating of limus active agent in microcrystalline form and tridodecanoylglycerol clearly lacked a uniform coating, the surface was uneven, and the coating easily crumbled off during inflation.

In attempts to coat medical devices with suspensions containing microcrystals of a limus active agent and tri-O-acylglycerols not according to the invention present in dissolved form in the suspension, which are fully esterified with the medium-length fatty acids or long-chain fatty acids not according to the invention as mentioned above, such as lauric acid, myristic acid or palmitic acid, it was not possible to produce coatings in which the microcrystals of the limus active agent are uniformly distributed on the medical device surface and adhere sufficiently to the medical device surface. For coatings with microcrystals of a limus active agent and a tri-O-acylglycerol not according to the invention, such as tridodecanoylglycerol, a higher particle release was observed in the “crumble test”, compared to coatings of microcrystals of a limus active agent and at least one tri-O-acylglycerol selected from the group consisting of trioctanoylglycerol, trinonanoylglycerol, tridecanoylglycerol, and triundecanoylglycerol. The comparison clearly showed that for the coating according to the invention with microcrystals of a limus active agent and at least one tri-O-acylglycerol selected from the group consisting of trioctanoylglycerol, trinonanoylglycerol, tridecanoylglycerol, and triundecanoylglycerol the particle release for all measured particle sizes is very far below the particle release for coatings with microcrystals of a limus active agent and a tri-O-acylglycerol not according to the invention, such as tridodecanoylglycerol.

It could be also surprisingly shown that outstanding adhesion of the microcrystals of the limus active agent to the medical device surface is achieved in particular when the tri-O-acylglycerol according to the invention is present in dissolved form in the crystal suspension according to the invention and is applied to the medical device surface together or simultaneously with the microcrystals of the suspended at least one limus active agent in the suspension.

This outstanding adhesion of the microcrystals of the limus active agent to the medical device surface could not be reproduced when the medical device surface was first coated with a solution containing at least one tri-O-acylglycerol according to the invention and when microcrystals of the limus active agent were subsequently applied to said tri-O-acylglycerol layer. Also, sufficient adhesion of the microcrystals of the limus active agent was not found when the medical device surface was first coated with crystals of the limus active agent according to the prior art methods and then coated with a solution containing a tri-O-acylglycerol in which the microcrystalline limus active agent does not dissolve.

In the prior art, the lack of adhesion of the microcrystals of the limus active agent to the medical device surface has been the major drawback for the use of microcrystalline limus active agents for coating of medical devices. The present invention overcomes this drawback and provides a solution for providing coatings with microcrystalline limus active agents on medical device surfaces.

In the suspension according to the invention, the at least one tri-O-acylglycerol selected from the group consisting of trioctanoylglycerol, trinonanoylglycerol, tridecanoylglycerol, and triundecanoylglycerol is advantageously present in dissolved form, so that when a medical device is coated with a suspension according to the invention, a coating is obtained in which not only the microcrystals of the limus active agent are uniformly distributed, but also the at least one tri-O-acylglycerol is uniformly distributed throughout the coating. This is particularly advantageous if a coating of microcrystals of a limus active agent is to be provided with a greater layer thickness and wherein the suspension according to the invention is applied several times in succession. Then, the tri-O-acylglycerols according to the invention are uniformly distributed in such a coating and hold the microcrystals of the limus active agent like “flexible adhesive” on the medical device surface, but also among the microcrystals like “flexible adhesive”.

Thus, the suspension of the present invention offers the additional advantage that the adhesion of the microcrystals of the at least one limus active agent is also increased among each other. If the microcrystals of the at least one limus active agent are first applied without a tri-O-acylglycerol dissolved in the suspension and, in a subsequent step, coated with a tri-O-acylglycerol solution, the tri-O-acylglycerol solution cannot sufficiently penetrate under and between the microcrystals of the limus active agent and thus cannot sufficiently provide the technical effect of increased adhesion on the medical device surface and between the microcrystals of the at least one limus active agent.

With the provision of a suspension according to the present invention containing at least one dissolved tri-O-acylglycerol selected from the group trioctanoylglycerol, trinonanoylglycerol, tridecanoylglycerol, and triundecanoylglycerol, and at least one limus active agent in the form of microcrystals, the major problem of poor adhesion of the microcrystals of the limus active agent to the medical device surface has now surprisingly been solved.

The coating of medical devices with microcrystalline limus active agents in the presence of at least one tri-O-acylglycerol selected from the group consisting of trioctanoylglycerol, trinonanoylglycerol, tridecanoylglycerol, and triundecanoylglycerol also shows significantly reduced “crumbling” behavior compared to medical devices currently on the market, since they show a significantly reduced particle release compared to the medical products currently available on the market, especially in the area of drug-delivering balloon catheters, thus proving that the stability of a coating according to the present invention is equally increased. This results in particular from the advantage that the at least one tri-O-acylglycerol selected from the groups consisting of trioctanoylglycerol, trinonanoylglycerol, tridecanoylglycerol, and triundecanoylglycerol, firmly holds the microcrystals of the limus active agent on the medical device surface like “flexible adhesive”, resulting in a stable, non-brittle and flexible coating.

In addition, it could be shown that tri-O-acylglycerols selected from the group consisting of trioctanoylglycerol, trinonanoylglycerol, tridecanoylglycerol, and triundecanoylglycerol, or mixtures of said tri-O-acylglycerols have other advantages. Among other things, they enable optimal transfer of the microcrystalline limus active agent into a tissue. The coatings prepared with the suspension according to the invention have a smoother, uniform surface and uniform distribution of the microcrystals of the limus active agent on the medical surface than is the case with the known coated medical products available on the market. These advantageous properties of the at least one tri-O-acylglycerols selected from the group consisting of trioctanoylglycerol, trinonanoylglycerol, tridecanoylglycerol, and triundecanoylglycerol thus lead to optimal active agent transfer and elution into the tissue, as well as optimal active agent distribution at the implantation site.

The tri-O-acylglycerols selected from the group consisting of trioctanoylglycerol, trinonanoylglycerol, tridecanoylglycerol, and triundecanoylglycerol, or mixtures of these tri-O-acylglycerols thus fulfill the following important tasks, among others: They are the microcrystalline limus active agent carriers and thus influence the mechanical properties of the coating (“drug carrier”) such as adhesion to the medical device surface, ensure the lowest possible loss of microcrystalline limus active agent during implantation (“drug transit loss”), but also influence the particle size distribution of the coating and thus the “crumbling behavior” (“particle release”), by which is meant the brittleness or pliability and adaptability of the coating before and during implantation and the associated change in shape. The uniformity of the coating (“uniformity”) is considered to be another important parameter, since a uniform coating can also achieve a uniform distribution of the limus active agent microcrystals on the medical device surface and thus also a uniform distribution of the microcrystalline limus active agent into the surrounding tissue. In addition, as a “catalyst”, they accelerate or facilitate the active agent transfer of the microcrystals of the limus active agent into the surrounding tissue without altering the microcrystalline limus active agent and influencing its efficacy (“drug transfer promoter”).

The tri-O-acylglycerols according to the invention selected from the group consisting of trioctanoylglycerol, trinonanoylglycerol, tridecanoylglycerol, and triundecanoylglycerol, or mixtures of said tri-O-acylglycerols are thus particularly advantageous for coatings of medical devices with microcrystals of a limus active agent, since the resulting coatings adhere to the surface of the medical device without loss, undergo changes in the shape of the substrate, e.g. elongations, without problems and without premature detachment or even dissolution, but do not prematurely “lose” the embedded or applied at least one microcrystalline limus active agent. Thereby, the coating does not suffer any damage even in case of severe shape changes, e.g. due to folding, expansion and deflation of such a coated balloon catheter.

Thus, the objective of the present invention is solved with respect to sufficient active agent adhesion and active agent release of the microcrystalline limus active agent via the suspension according to the invention, which contains at least one tri-O-acylglycerol selected from the group consisting of trioctanoylglycerol, trinonanoylglycerol, tridecanoylglycerol, and triundecanoylglycerol in dissolved form.

The term “trioctanoylglycerol” or “tri-O-octanoylglycerol”, as used herein, refers to a tri-O-acylglycerol in which the glycerol is fully esterified with caprylic acid or octanoic acid. Synonymous names for trioctanoylglycerol known in the prior art are trioctanoylglyceride, glycerin trioctanoin, tricapryloylglycerin, octanoic acid-1,1′,1″-(1,2,3-propanetriyl)ester, glycerin tricaprylate, tricaprylyl glycerin, TG(8:0/8:0/8:0), glycerol tricaprylate, caprylic acid-1,2,3-propanetriyl ester, caprylin, octanoic acid triglyceride, tricapryl glyceride, tricaprylin, trioctanoyl glycerol, octanoic acid-1,2,3-propanetriyl ester, glycerol trioctanoate, 1,2,3-propanetriol trioctanoate, caprylic acid triglyceride, glycerol tricaprylate, caprylic triglyceride, tricaprilin, tricaprylyl glycerol, 1,2,3-trioctanoyl glycerol, glycerol trioctanoate and 1,2,3-tricapryloyl glycerol. Trioctanoylglycerol has CAS number 538-23-8, a molecular weight of 470.68 g/mol, and has the following structural formula:

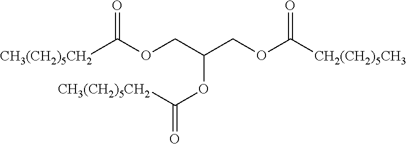

Octanoic acid is a carboxylic acid known by the trivial name caprylic acid and is a saturated fatty acid of the following structural formula:

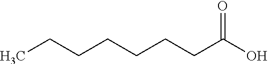

In preferred embodiments of the present invention, the suspension for coating of a medical device, preferably selected from a catheter balloon, a balloon catheter, a stent, or a cannula, comprises at least trioctanoylglycerol. In preferred embodiments of the present invention, the at least one tri-O-acylglycerol is thus preferably selected from trioctanoylglycerol.

The melting point of trioctanoylglycerol is in the range of 9-10° C. Trioctanoylglycerol exists under normal conditions (20° C., 101 hPa) as an odorless clear colorless to amber liquid. Trioctanoylglycerol is practically insoluble in water.

The term “trinonanoylglycerol” or “tri-O-nonanoylglycerol”, as used herein, refers to a tri-O-acylglycerol in which the glycerol is fully esterified with pelargonic acid or nonanoic acid. Synonymous names for tridecanoylglycerol known in the prior art are glycerol tripelargonate, trinonanoin, 1,2,3-trinonanoylglycerol, tripleargonin, 1,2,3-tripelargonoyl-glycerol. Trinonanoylglycerol has CAS number 126-53-4, a molecular weight of 512.76 g/mol and has the following structural formula:

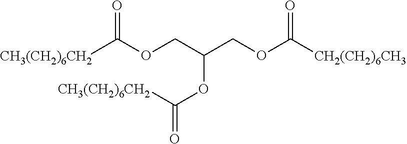

Nonanoic acid is a carboxylic acid known by the trivial name pelargonic acid and is a saturated fatty acid of the following structural formula:

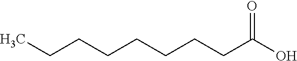

In preferred embodiments of the present invention, the suspension for coating of preferably selected from a catheter balloon, a balloon catheter, a stent, or a cannula, comprises at least trinonanoylglycerol. In preferred embodiments of the present invention, the at least one tri-O-acylglycerol is thus preferably selected from trinonanoylglycerol.

The melting point of trinonanoylglycerol is in the range of 8-9° C. Trinonanoylglycerol exists as a liquid under normal conditions (20° C., 101 hPa). Trinonanoylglycerol is practically insoluble in water.

The term “tridecanoylglycerol” or “tri-O-decanoylglycerol”, as used herein, refers to a tri-O-acylglycerol in which the glycerol is fully esterified with capric acid or decanoic acid. Synonymous names for tridecanoylglycerol known in the prior art are glycerol tris-(decanoate), 1,2,3-tricaprinoyl-glycerol, tricaprin, 1,2,3-tridecanoylglycerol, glycerol tridecanoate, tridecanoin. Tridecanoylglycerol has CAS number 621-71-6, a molecular weight of 554.84 g/mol, and has the following structural formula:

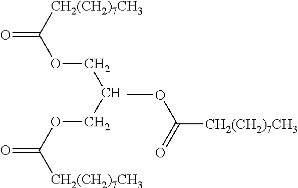

Decanoic acid is a carboxylic acid known by the trivial name capric acid and is a saturated fatty acid of the following structural formula:

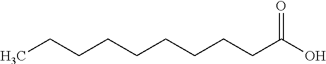

In preferred embodiments of the present invention, the suspension for coating of preferably selected from a catheter balloon, a balloon catheter, a stent, or a cannula, comprises at least tridecanoylglycerol. In preferred embodiments of the present invention, the at least one tri-O-acylglycerol is thus preferably selected from tridecanoylglycerol.

The melting point of tridecanoylglycerol is in the range of 31-33° C. Tridecanoylglycerol exists as a pale yellow solid under normal conditions (20° C., 101 hPa). Tridecanoylglycerol is practically insoluble in water.

The term “triundecanoylglycerol” or “tri-O-undecanoylglycerol”, as used herein, refers to a tri-O-acylglycerol in which the glycerol is fully esterified with undecanoic acid. Synonymous names for triundecanoylglycerol known in the prior art are glycerol triundecanoate, triundecanoin, 1,2,3-triundecanoylglycerol, triundecanin. The IUPAC name is 1,3-bis(undecanoyloxy)propan-2-yl-undecanoate. Triundecanoylglycerol has the CAS number 13552-80-2, a molecular weight of 596.9 g/mol, and has the following structural formula:

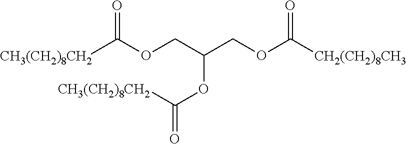

Undecanoic acid is a saturated fatty acid with the following structural formula:

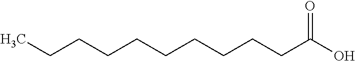

In preferred embodiments of the present invention, the suspension for coating of a medical device, preferably selected from a catheter balloon, a balloon catheter, a stent, or a cannula, comprises at least triundecanoylglycerol. In preferred embodiments of the present invention, the at least one tri-O-acylglycerol is thus preferably selected from triundecanoylglycerol.

The melting point of triundecanoylglycerol is in the range of 30-32° C. Triundecanoylglycerol exists as a solid under normal conditions (20° C., 101 hPa). Triundecanoylglycerol is practically insoluble in water.

Thus, the suspension of the present invention for coating of a medical device, preferably selected from a catheter balloon, a balloon catheter, a stent, or a cannula, contains at least one tri-O-acylglycerol selected from the group consisting of trioctanoylglycerol, trinonanoylglycerol, tridecanoylglycerol, and triundecanoylglycerol according to the invention.

In other words, the suspension of the present invention for coating of a medical device, preferably selected from a catheter balloon, a balloon catheter, a stent, or a cannula, contains according to the invention at least one tri-O-acylglycerol selected from tri-O-octanoylglycerol, tri-O-nonanoylglycerol, tri-O-decanoylglycerol, and tri-O-undecanoylglycerol.

With other words, the suspension of the present invention for coating of a medical device, preferably selected from a catheter balloon, a balloon catheter, a stent, or a cannula, contains according to the invention either a tri-O-acylglycerol selected from the group consisting of trioctanoylglycerol, trinonanoylglycerol, tridecanoylglycerol, and triundecanoylglycerol, or a mixture of at least two tri-O-acylglycerols selected from the group consisting of trioctanoylglycerol, trinonanoylglycerol, tridecanoylglycerol, and triundecanoylglycerol.

Still differently formulated, the suspension of the present invention for coating of a medical device, preferably selected from a catheter balloon, a balloon catheter, a stent, or a cannula, contains according to the invention either a tri-O-acylglycerol selected from the group consisting of trioctanoylglycerol, trinonanoylglycerol, tridecanoylglycerol, and triundecanoylglycerol, or a mixture of two, three or four tri-O-acylglycerols selected from the group consisting of trioctanoylglycerol, trinonanoylglycerol, tridecanoylglycerol, and triundecanoylglycerol.

Thus, the phrase “at least one tri-O-acylglycerol selected from the group consisting of trioctanoylglycerol, trinonanoylglycerol, tridecanoylglycerol, and triundecanoylglycerol” herein also refers to mixtures of the tri-O-acylglycerol selected from the group consisting of trioctanoylglycerol, trinonanoylglycerol, tridecanoylglycerol, and triundecanoylglycerol. Thus, the phrase “at least one tri-O-acylglycerol” includes wording such as “at least two tri-O-acylglycerols” “at least three tri-O-acylglycerol” “two tri-O-acylglycerols” “three tri-O-acylglycerols” and “four tri-O-acylglycerols”.

In some embodiments of the present invention, the suspension for coating of medical devices preferably selected from a catheter balloon, a balloon catheter, a stent, or a cannula, contains at least two tri-O-acylglycerols selected from the group consisting of trioctanoylglycerol, trinonanoylglycerol, tridecanoylglycerol, and triundecanoylglycerol.

In some embodiments of the present invention, the suspension for coating of medical devices preferably selected from a catheter balloon, a balloon catheter, a stent, or a cannula, contains at least three tri-O-acylglycerols selected from the group consisting of trioctanoylglycerol, trinonanoylglycerol, tridecanoylglycerol, and triundecanoylglycerol.

In some embodiments of the present invention, the suspension for coating medical devices preferably selected from a catheter balloon, a balloon catheter, a stent, or a cannula, contains trioctanoylglycerol, trinonanoylglycerol, tridecanoylglycerol, and triundecanoylglycerol. The tri-O-acylglycerols selected from the group consisting of trioctanoylglycerol, trinonanoylglycerol, tridecanoylglycerol, and triundecanoylglycerol according to the invention have a melting point below 37° C., so they are present in molten form at body temperature or melt or soften at body temperature.

In some embodiments of the present invention, it is preferred that tri-O-acylglycerols present as a liquid under normal conditions (20° C., 101 hPa), such as trioctanoylglycerol or trinonanoylglycerol, especially preferably trioctanoylglycerol, are used to prepare the suspensions according to the invention. Especially with trioctanoylglycerol excellent stable, non-brittle and flexible coatings could be obtained. In some embodiments, mixtures of trioctanoylglycerol, trinonanoylglycerol, tridecanoylglycerol, and triundecanoylglycerol containing at least trioctanoylglycerol and/or trinonanoylglycerol, particularly preferably trioctanoylglycerol, are also preferred, since the resulting mixtures are present as a liquid under normal conditions (20° C., 101 hPa). A particularly preferred mixture of tri-O-acylglycerols herein is a mixture of trioctanoylglycerol and tridecanoylglycerol.

In some further embodiments of the present invention, it is preferred that tri-O-acylglycerols present as solids under normal conditions (20° C., 101 hPa), such as tridecanoylglycerol, or triundecanoylglycerol, are used to prepare the suspensions according to the invention. Especially with tridecanoylglycerol, excellent stable, non-friable and flexible coatings could be obtained. Since body temperature is still higher than the melting points of these tri-O-acylglycerols, tridecanoylglycerol, or triundecanoylglycerol are present in molten form at body temperature during implantation, so that especially during inflation of medical devices, such as catheter balloons or stents, there are no disadvantages with regard to particle release and thus friability compared to tri-O-acylglycerols that are present as a liquid under normal conditions (20° C., 101 hPa), such as trioctanoylglycerol or trinonanoylglycerol.

In addition, if necessary or desired, the coated medical device can also be heated prior to implantation so that the tridecanoylglycerol, or triundecanoylglycerol melts or softens prior to implantation and is thus already in molten form at the beginning of implantation.

In some preferred embodiments of the present invention, the suspension for coating of a medical device, preferably selected from a catheter balloon, a balloon catheter, a stent, or a cannula thus contains at least one tri-O-acylglycerol selected from the group consisting of trioctanoylglycerol, trinonanoylglycerol, tridecanoylglycerol, and triundecanoylglycerol, further preferably at least one tri-O-acylglycerol selected from the group consisting of trioctanoylglycerol, trinonanoylglycerol and tridecanoylglycerol, still further preferably at least one tri-O-acylglycerol selected from trioctanoylglycerol and tridecanoylglycerol, further preferably at least one tri-O-acylglycerol selected from the group consisting of trioctanoylglycerol or at least one tri-O-acylglycerol selected from tridecanoylglycerol.

In some further preferred embodiments of the present invention, the suspension for coating of a medical device preferably selected from a catheter balloon, a balloon catheter, a stent, or a cannula contains a mixture of tri-O-acylglycerols selected from the group consisting of trioctanoylglycerol, trinonanoylglycerol, tridecanoylglycerol, and triundecanoylglycerol. In some of these preferred embodiments, the suspension for coating of medical devices preferably selected from a catheter balloon, a balloon catheter, a stent, or a cannula contains a mixture of trioctanoylglycerol, trinonanoylglycerol and tridecanoylglycerol, or a mixture of trioctanoylglycerol, trinonanoylglycerol and triundecanoylglycerol, or a mixture of trioctanoylglycerol, tridecanoylglycerol, and triundecanoylglycerol, or a mixture of trinonanoylglycerol, tridecanoylglycerol, and triundecanoylglycerol, further preferably a mixture of trioctanoylglycerol, trinonanoylglycerol and tridecanoylglycerol, or a mixture of trioctanoylglycerol, tridecanoylglycerol, and triundecanoylglycerol, still more preferably a mixture of trioctanoylglycerol, trinonanoylglycerol and tridecanoylglycerol.

In some preferred embodiments, the suspension for coating a of medical device, preferably selected from a catheter balloon, a balloon catheter, a stent or a cannula, contains a mixture of trioctanoylglycerol and tridecanoylglycerol, or a mixture of trioctanoylglycerol and trinonanoylglycerol, or a mixture of trioctanoylglycerol and triundecanoylglycerol, or a mixture of trinonanoylglycerol and tridecanoylglycerol, or a mixture of trinonanoylglycerol and triundecanoylglycerol, or a mixture of tridecanoylglycerol, and triundecanoylglycerol.

In some preferred embodiments of the present invention, the suspension for coating of a medical device, preferably selected from a catheter balloon, a balloon catheter, a stent, or a cannula, contains a mixture of tridecanoylglycerol, and trinonanoylglycerol, or a mixture of tridecanoylglycerol, and triundecanoylglycerol, or a mixture of tridecanoylglycerol, and trioctanoylglycerol, further preferably a mixture of tridecanoylglycerol, and trinonanoylglycerol, or a mixture of tridecanoylglycerol, and trioctanoylglycerol, and most preferably a mixture of tridecanoylglycerol, and trioctanoylglycerol.

In some preferred embodiments of the present invention, the suspension for coating of a medical device, preferably selected from a catheter balloon, a balloon catheter, a stent, or a cannula, contains a mixture of trioctanoylglycerol and trinonanoylglycerol, or a mixture of trioctanoylglycerol and triundecanoylglycerol, or a mixture of trioctanoylglycerol and tridecanoylglycerol, further preferably a mixture of trioctanoylglycerol and trinonanoylglycerol, or a mixture of trioctanoylglycerol and tridecanoylglycerol, and most preferably a mixture of trioctanoylglycerol and tridecanoylglycerol.

In most preferred embodiments of the present invention, the suspension for coating of a medical device, preferably selected from a catheter balloon, a balloon catheter, a stent, or a cannula, contains at least one tri-O-acylglycerol selected from the group consisting of trioctanoylglycerol and tridecanoylglycerol, or a mixture of trioctanoylglycerol and tridecanoylglycerol.

In some preferred embodiments of the present invention, the suspension for coating of a medical device, preferably selected from a catheter balloon, a balloon catheter, a stent, or a cannula, contains at least one tri-O-acylglycerol selected trioctanoylglycerol, or a mixture of trioctanoylglycerol and at least one tri-O-acylglycerol selected from the group consisting of trinonanoylglycerol, tridecanoylglycerol, and triundecanoylglycerol, preferably trinonanoylglycerol and tridecanoylglycerol, even more preferably tridecanoylglycerol.

In some preferred embodiments of the present invention, the suspension for coating of a medical device, preferably selected from a catheter balloon, a balloon catheter, a stent, or a cannula, contains trioctanoylglycerol, or a mixture of trioctanoylglycerol and at least one further tri-O-acylglycerol selected from trinonanoylglycerol, tridecanoylglycerol, and triundecanoylglycerol, preferably trinonanoylglycerol and tridecanoylglycerol, even more preferably tridecanoylglycerol.

In some preferred embodiments of the present invention, the suspension for coating of a medical device, preferably selected from a catheter balloon, a balloon catheter, a stent, or a cannula, contains at least one tri-O-acylglycerol selected from tridecanoylglycerol, or a mixture of tridecanoylglycerol, and at least one further tri-O-acylglycerol selected from the group consisting of trinonanoylglycerol, trioctanoylglycerol and triundecanoylglycerol, preferably trinonanoylglycerol and trioctanoylglycerol, even more preferably trioctanoylglycerol.

In some preferred embodiments of the present invention, the suspension for coating a medical device, preferably selected from a catheter balloon, a balloon catheter, a stent, or a cannula, contains tridecanoylglycerol, or a mixture of tridecanoylglycerol, and at least one further tri-O-acylglycerol selected from the group consisting of trinonanoylglycerol, trioctanoylglycerol and triundecanoylglycerol, preferably trinonanoylglycerol and trioctanoylglycerol, even more preferably trioctanoylglycerol.

The at least one tri-O-acylglycerol selected from the group consisting of trioctanoylglycerol, trinonanoylglycerol, tridecanoylglycerol, and triundecanoylglycerol preferably has at least a purity of ≥90%, preferably ≥95%, and more preferably ≥99%. A mixture of tri-O-acylglycerols selected from trioctanoylglycerol, trinonanoylglycerol, tridecanoylglycerol, and triundecanoylglycerol preferably has at least ≥90%, preferably ≥95% and more preferably ≥99% of the tri-O-acylglycerols selected from the group consisting of trioctanoylglycerol, trinonanoylglycerol, tridecanoylglycerol, and triundecanoylglycerol.

Particularly preferably, the suspension according to the invention does not contain any other tri-O-acylglycerols in addition to the at least one tri-O-acylglycerol selected from the group consisting of trioctanoylglycerol, trinonanoylglycerol, tridecanoylglycerol, and triundecanoylglycerol.

For example, trioctanoylglycerol and tridecanoylglycerol are also present as natural components in various vegetable oils, such as soybean oil, olive oil or coconut oil, or animal oils. However, these natural vegetable oils or animal oils also contain other saturated and also unsaturated tri-O-acylglycerols in various proportions not according to the invention or also other substances such as mono-O-acylglycerols, di-O-acylglycerols, fatty acids and lipids, so that natural vegetable oils or animal oils are not suitable for the production of crystal suspensions according to the invention. Vegetable oils or animal oils can be used for the preparation of crystal suspension according to the invention, provided that they consist of at least ≥90%, preferably ≥95% and particularly preferably of ≥99% of at least one tri-O-acylglycerol selected from the group consisting of trioctanoylglycerol, trinonanoylglycerol, tridecanoylglycerol, and triundecanoylglycerol.

Mixtures of tri-O-acylglycerols not according to the invention are therefore herein oils such as linseed oil, hemp oil, corn oil, walnut oil, rapeseed oil, soybean oil, sunflower oil, poppy seed oil, safflower oil, wheat germ oil, safflower oil, grape seed oil, evening primrose oil, borage oil, black cumin oil, algae oil, fish oil, cod liver oil, coconut oil and/or mixtures of the aforementioned oils.

For the preparation of the suspension according to the invention containing at least one tri-O-acylglycerol selected from the group consisting of trioctanoylglycerol, trinonanoylglycerol, tridecanoylglycerol, and triundecanoylglycerol, the at least one tri-O-acylglycerol is either used as a chemically pure substance for the preparation of the crystal suspension according to the invention or a mixture is used, that consists of at least ≥90%, preferably ≥95% and more preferably ≥99% of at least one tri-O-acylglycerol selected from the group consisting of trioctanoylglycerol, trinonanoylglycerol, tridecanoylglycerol, and triundecanoylglycerol. The at least one tri-O-acylglycerol selected from the group consisting of trioctanoylglycerol, trinonanoylglycerol, tridecanoylglycerol, and triundecanoylglycerol can of course also be obtained from a natural vegetable oil or animal oil, e.g. the tri-O-acylglycerols trioctanoylglycerol and tridecanoylglycerol, and mixtures of trioctanoylglycerol and tridecanoylglycerol, are commercially available under the trade names Captex® 8000 (trioctanoylglycerol), Captex® 1000 (tridecanoylglycerol), Captex® 300 (trioctanoylglycerol/tridecanoylglycerol), Captex® 355 (trioctanoylglycerol/tridecanoylglycerol), Miglyol® 810 (trioctanoylglycerol: Tridecanoylglycerol, approx. 70:30), and Miglyol® 812 (trioctanoylglycerol: tridecanoylglycerol, approx. 50:50). Such commercially available mixtures can be used to prepare a suspension of the present invention.

In some preferred embodiments of the present invention, the suspension for coating of a medical device, preferably selected from a catheter balloon, a balloon catheter, a stent, or a cannula, thus contains a mixture of at least two tri-O-acylglycerols selected from the group consisting of trioctanoylglycerol, trinonanoylglycerol, tridecanoylglycerol, and triundecanoylglycerol, wherein the mixture consists of at least ≥90%, preferably ≥95%, and more preferably ≥99% of at least two tri-O-acylglycerols selected from the group consisting of trioctanoylglycerol, trinonanoylglycerol, tridecanoylglycerol, and triundecanoylglycerol.

The term “weight percent” (abbreviation: wt. %), as used herein, refers to the proportion of a substance in a mixture or solution measured in grams per 100 g of mixture. The term weight percent, as used herein, is a designation for the mass fraction of a mixture.

The term “mass fraction”, as used herein, refers to a physicochemical quantity for the quantitative description of the composition of mixtures of substances. The mass of a considered mixture component is related to the sum of the masses of all mixture components, the mass fraction indicates the relative proportion of the mass of a considered mixture component in the total mass of the mixture.

In preferred embodiments, the mass fraction (m/m; [g]/[g]; m=mass) of tri-O-acylglycerol to the total mass of tri-O-acylglycerol and microcrystalline limus active agent in the suspension is preferably 5-40%, more preferably 10-30% and most preferably 20%. The mass of tri-O-acylglycerol here refers to the total mass of tri-O-acylglycerol selected from the group consisting of trioctanoylglycerol, trinonanoylglycerol, tridecanoylglycerol and triundecanoylglycerol, i.e. in the case of mixtures of tri-O-acylglycerols selected from trioctanoylglycerol, trinonanoylglycerol, tridecanoylglycerol, and triundecanoylglycerol, the mass fraction of the mass of the mixture is calculated from the total mass of tri-O-acylglycerol mixture and microcrystalline limus active agent.

Thus, to calculate the mass fraction of tri-O-acylglycerol to the total mass of tri-O-acylglycerol and microcrystalline limus active agent in the suspension, the quotient of the mass of tri-O-acylglycerol and the total mass of tri-O-acylglycerol and microcrystalline limus active agent is formed. For example, the mass fraction of tri-O-acylglycerol in a mixture of 4 g of limus active agent and 1 g of tri-O-acylglycerol is 20%.

In preferred embodiments, the mass fraction (m/m; [g]/[g]; m=mass) of tri-O-acylglycerol to the total mass of tri-O-acylglycerol and microcrystalline limus active agent in the suspension is preferably 0.1-50%, further preferably 1-40%, more preferably 10-30%, and most preferably 20%.

In preferred embodiments, the mass fraction (m/m; [g]/[g]; m=mass) of limus active agent to the total mass of tri-O-acylglycerol and microcrystalline limus active agent in the suspension is preferably 99.9-50%, further preferably 99-60%, more preferably 90-70%, and most preferably 80%.

Thus, the mass fractions of tri-O-acylglycerol and microcrystalline limus active agent to the total mass of tri-O-acylglycerol and microcrystalline limus active agent are preferably 0.1-50% tri-O-acylglycerol and 99.9-50% limus active agent, further preferably 1-40% tri-O-acylglycerol and 99-60% limus active agent, more preferably 10-30% tri-O-acylglycerol and 90-70% limus active agent and most preferably 20% tri-O-acylglycerol and 80% limus active agent.

If mixtures of tri-O-acylglycerols are used, the mass fraction of the mass of tri-O-acylglycerol mixture to the total mass of tri-O-acylglycerol mixture and microcrystalline limus active agent is determined. For example, the mass fraction of tri-O-acylglycerol mixture in a mixture of 4 g limus active agent and tri-O-acylglycerol mixture is 20%.

In preferred embodiments, the amount of tri-O-acylglycerol relative to the limus active agent is preferably 0.1-50 wt. %, further preferably 1-40 wt. %, more preferably 10-30 wt. %, and most preferably 20 wt. %.

In preferred embodiments, the amount of limus active agent relative to the tri-O-acylglycerol is preferably 99.9-50 wt. %, further preferably 99-60 wt. %, more preferably 90-70 wt. % and most preferably 80 wt. % in the suspension.

Thus, the tri-O-acylglycerol and the microcrystalline limus active agent in the suspension are preferably present at 0.1-50 wt. % tri-O-acylglycerol to 99.9-50 wt. % limus active agent, further preferably at 1-40 wt. % tri-O-acylglycerol to 99.60 wt. % limus active agent, particularly preferably with 10-30 wt. % tri-O-acylglycerol to 90-70 wt. % limus active agent and most preferably with 20 wt. % tri-O-acylglycerol and 80 wt. % limus active agent.

In preferred embodiments, the mass fraction (m/m; [g]/[g]; m=mass) of antioxidants to the total mass of antioxidants and microcrystalline limus active agent in the suspension is preferably 0.001-15.0%, further preferably 0.01-10.0%, more preferably 0.05-5.0%.

In preferred embodiments, the mass fraction (m/m; [g]/[g]; m=mass) of additives other than antioxidants in the total mass of additives and microcrystalline limus active agent in the suspension is preferably 0.001-1.0%, further preferably 0.01-2.5%, more preferably particularly preferably 0.05-5.0%.

In preferred embodiments, the mass fraction (m/m; [g]/[g]; m=mass) of antioxidants together with other additives that are not antioxidants in the total mass of antioxidants and the additives and microcrystalline limus active agent in the suspension is preferably 0.001-15.0%, further preferably 0.01-10.0%, particularly preferably 0.05-5.0%.

In preferred embodiments, the mass fraction (m/m; [g]/[g]; m=mass) of antioxidants together with further additives other than antioxidants in the total mass of antioxidants and the additives and microcrystalline limus active agent in the suspension is preferably 0.001-15.0%, further preferably 0.01-10.0%, particularly preferably 0.05-5.0%, wherein the mass fraction (m/m; [g]/[g]; m=mass) of additives other than antioxidants to the total mass of additives and microcrystalline limus active agent in the suspension is preferably 0.001-1.0%, further preferably 0.01-2.5%, more preferably particularly preferably 0.05-5.0%.

The term “mass ratio”, as used herein, refers to a physicochemical quantity for the quantitative description of the composition of mixtures of substances. The mass ratio indicates the ratio of the masses of two considered mixture components to each other.

The mass ratio (m/m; [g]/[g]; m=mass) of tri-O-acylglycerol to microcrystalline limus active agent is preferably 1:1000-1:1, more preferably 1:100-1:1.5, further preferably 1:10-1:3, and more preferably 1:5-1:4, and most preferably 1:4. The mass of tri-O-acylglycerol here refers to the total mass of tri-O-acylglycerol selected from the group consisting of trioctanoylglycerol, trinonanoylglycerol, tridecanoylglycerol, and triundecanoylglycerol, i.e. for mixtures of tri-O-acylglycerols selected from the group consisting of trioctanoylglycerol, trinonanoylglycerol, tridecanoylglycerol, and triundecanoylglycerol, the mass of the tri-O-acylglycerol mixture is used to calculate the mass ratio.

Thus, to calculate the mass ratio of tri-O-acylglycerol to microcrystalline limus active agent, the quotient of the mass of tri-O-acylglycerol and the mass of limus active agent is formed. For example, the mass ratio of tri-O-acylglycerol to microcrystalline limus active agent in a mixture of 1 g tri-O-acylglycerol and 4 g limus active agent is 1:4.

The mass ratio of tri-O-acylglycerol to microcrystalline limus active agent is further preferably 0.1-50%, more preferably 1-40%, more preferably 10-30%, and further preferably 20-25%, and most preferably 25%. In some preferred embodiments, the mass ratio of tri-O-acylglycerol to microcrystalline limus active agent is preferably 5-40%, more preferably 10-30%, and most preferably 20-25%.

Thus, to calculate the mass ratio of tri-O-acylglycerol to microcrystalline limus active agent, the quotient of the mass of tri-O-acylglycerol and the mass of limus active agent is formed. For example, the mass ratio of tri-O-acylglycerol to microcrystalline limus active agent in a mixture of 1 g tri-O-acylglycerol and 4 g limus active agent is 25%.

In preferred embodiments, the mass ratio (m/V; [g]/[100 mL]; m=mass; V=volume) of microcrystalline limus active agent to 100 mL suspension volume is 0.5-6%, more preferably 1-5%, even more preferably 2-4%, and most preferably 3%.

Thus, to calculate the mass ratio of microcrystalline limus active agent to 100 mL suspension volume, the quotient of the mass of microcrystalline limus active agent and 100 mL suspension volume is formed. For example, the mass ratio of microcrystalline limus active agent to 100 mL suspension volume in a suspension of 3 g limus active agent and 100 mL suspension volume is 3%.

Based on a volume of 1 L suspension volume, the mass ratio (m/V; [g]/[L]; m=mass; V=volume) of microcrystalline limus active agent to 1 L suspension volume is 0.5-6%, more preferably 1-5%, still more preferably 2-4%, and most preferably 3%.

Thus, to calculate the mass ratio of microcrystalline limus active agent to 1 L suspension volume, the quotient of the mass of microcrystalline limus active agent to 1 L suspension volume is formed. For example, the mass ratio of microcrystalline limus active agent to 1 L suspension volume in a suspension of 30 g limus active agent and 1 L suspension volume is 3%.

In preferred embodiments, the mass ratio (m/V; [g]/[100 mL]; m=mass; V=volume) of tri-O-acylglycerol to 100 mL suspension volume is 0.13-1.5%, more preferably 0.25-1.25%, even more preferably 0.5-1%, and most preferably 0.75%.

Thus, to calculate the mass ratio of tri-O-acylglycerol to 100 mL suspension volume, the quotient of the mass of tri-O-acylglycerol and 100 mL suspension volume is formed. For example, the mass ratio of tri-O-acylglycerol to 100 mL suspension volume in a suspension of 0.75g tri-O-acylglycerol and 100 mL suspension volume is 0.75%.

In other words, the mass ratio (m/V; [g]/[L]; m=mass; V=volume) of tri-O-acylglycerol to 1 L suspension volume is 0.13-1.5%, more preferably 0.25-1.25%, even more preferably 0.5-1% and most preferably 0.75%. Thus, to calculate the mass ratio of tri-O-acylglycerol to 1 L suspension volume, the quotient of the mass of tri-O-acylglycerol and 1 L suspension volume is formed. For example, the mass ratio of tri-O-acylglycerol to 1 L suspension volume in a suspension of 7.5g limus active agent and 1 L suspension volume is 0.75%.

The term “mass concentration”, as used herein, refers to a physicochemical quantity for the quantitative description of the composition of substance mixtures/mixed phases. Here, the mass of a considered mixture component is related to the total volume of the mixture phase.

In preferred embodiments, the mass concentration (m/V; [mg]/[mL]; m=mass; V=volume) of microcrystalline limus active agent in the suspension is preferably 5-60 mg/mL, more preferably 20-40 mg/mL, further preferably 20-30 mg/mL. In some preferred embodiments, the mass concentration of microcrystalline limus active agent in the suspension is 20-25 mg/mL. In some preferred embodiments, the mass concentration of microcrystalline limus active agent in the suspension is 25-30 mg/mL. In some preferred embodiments, the mass concentration of microcrystalline limus active agent in the suspension is 20 mg/mL. In some preferred embodiments, the mass concentration of microcrystalline limus active agent in the suspension is 25 mg/mL. In some preferred embodiments, the mass concentration of microcrystalline limus active agent in the suspension is 30 mg/mL.

In other words, the mass concentration (m/V; [kg]/[m³]; m=mass; V=volume) of microcrystalline limus active agent in the suspension is preferably 5-60 kg/m³, more preferably 20-40 kg/m³, more preferably 20-30 kg/m³. In some preferred embodiments, the mass concentration of microcrystalline limus active agent in the suspension is 20-25 kg/m³. In some preferred embodiments, the mass concentration of microcrystalline limus active agent in the suspension is 25-30 kg/m³. In some preferred embodiments, the mass concentration of microcrystalline limus active agent in the suspension is 20 kg/m³. In some preferred embodiments, the mass concentration of microcrystalline limus active agent in the suspension is 25 kg/m³. In some preferred embodiments, the mass concentration of microcrystalline limus active agent in the suspension is 30 kg/m³.

In preferred embodiments, the mass concentration (m/V; [mg]/[mL]; m=mass; V=volume) of t tri-O-acylglycerol in the suspension is preferably 1.3-15 mg/mL, more preferably 2.5-12.5 mg/mL, more preferably 5-10 mg/mL.

In some preferred embodiments, the mass concentration of tri-O-acylglycerol in the suspension is 6-9 mg/mL. In some preferred embodiments, the mass concentration of tri-O-acylglycerol in the suspension is 5.5-9.5 mg/mL. In some preferred embodiments, the mass concentration of tri-O-acylglycerol in the suspension is 7.5 mg/mL. In some preferred embodiments, the mass concentration of tri-O-acylglycerol in the suspension is 7 mg/mL. In some preferred embodiments, the mass concentration of tri-O-acylglycerol in the suspension is 6.5 mg/mL.

In some preferred embodiments, the mass concentration of tri-O-acylglycerol in the suspension is 4-6 mg/mL. In some preferred embodiments, the mass concentration of tri-O-acylglycerol in the suspension is 4.5-5.5 mg/mL. In some preferred embodiments, the mass concentration of tri-O-acylglycerol in the suspension is 4.5 mg/mL. In some preferred embodiments, the mass concentration of tri-O-acylglycerol in the suspension is 5 mg/mL. In some preferred embodiments, the mass concentration of tri-O-acylglycerol in the suspension is 5.5 mg/mL.

In preferred embodiments, the mass concentration (m/V; [mg]/[mL]; m=mass; V=volume) of microcrystalline limus active agent in the suspension is 5-60 mg/mL and of tri-O-acylglycerol 1.3-15 mg/mL, further preferred is a mass concentration of microcrystalline limus active agent in the suspension of 20-40 mg/mL and of tri-O-acylglycerol 2.5-12.5 mg/mL, further preferred is a mass concentration of microcrystalline limus active agent in the suspension of 20-30 mg/mL and of tri-O-acylglycerol 5-10 mg/mL.

The term “amount of substance concentration”, as used herein, refers to a physicochemical quantity for the quantitative description of the composition of substance mixtures/mixed phases. Here, the amount of substance of a considered mixture component is related to the total volume of the mixed phase.

In preferred embodiments, the molar concentration (n/V; [mmol]/[L]; n=amount of substance; V=volume) of microcrystalline limus active agent in the suspension is preferably 5-60 mmol/L, more preferably 20-40 mmol/L, more preferably 25-35 mmol/L.

In other words, the molar concentration (n/V; [mmol]/[L]; n=amount of substance; V=volume) of microcrystalline limus active agent in the suspension is preferably 5-60 mol/m³, more preferably 20-40 mol/m³, more preferably 25-35 mol/m³.

In some preferred embodiments, the molar concentration (n/V; [mmol]/[L]; n=amount of substance; V=volume) of tri-O-acylglycerol in the suspension is 2-35 mmol/L, more preferably 4-25 mmol/L, more preferably 10-20 mmol/L, more preferably 12-18 mmol/L.

In some preferred embodiments, the molar concentration (n/V; [mmol]/[L]; n=amount of substance; V=volume) of tri-O-acylglycerol the suspension is 2-35 mol/m³, more preferably 4-25 mol/m³, more preferably 10-20 mol/m³, more preferably 12-18 mol/m³.

The term “limus active agent” refers to the group of macrolide lactones comprising the active agents rapamycin (sirolimus) and rapamycin derivatives such as everolimus, umirolimus (Biolimus©), deforolimus, myolimus, novolimus, pimecrolimus, ridaforolimus, tacrolimus, temsirolimus, and zotarolimus.

According to the invention, the following substances can be used as limus active agents: Rapamycin, deforolimus, myolimus, novolimus, 28-O-methylrapamycin, C-22-methylrapamycin, C-49-methylrapamycin, 42-O-(2-ethoxyethyl)-rapamycin (Umirolimus, Biolimus® or Biolimus A9®), 40-O-(2-hydroxyethyl)rapamycin (Everolimus), 40-O-benzylrapamycin, 40-O-(4′-hydroxymethyl)benzylrapamycin, 40-O-[4′-(1,2-dihydroxyethyl)] benzylrapamycin, 40-O-allylrapamycin, 40-O-[3′-(2,2-dimethyl-1,3-dioxolan-4(S)-yl)-prop-2′-en-1′-yl]-rapamycin, (2′:E,4′S)-40-O-(4′,5′-dihydroxypent-2′-en-1′-yl)-rapamycin 40-O-(2-hydroxy)ethoxycarbonylmethylrapamycin, 40-O-(3-hydroxy)propylrapamycin 40-O-(6-hydroxy)hexylrapamycin 40-O-[2-(2-hydroxy)ethoxy]ethylrapamycin 40-O-[(3S)-2,2-dimethyldioxolan-3-yl]methylrapamycin, 40-O-[(2S)-2,3-dihydroxyprop-1-yl]-rapamycin, 40-O-(2-acetoxy)ethylrapamycin 40-O-(2-nicotinoyloxy)ethylrapamycin, 40-O-[2-(N-morpholino)acetoxy]ethylrapamycin 40-O-(2-N-imidazolylacetoxy)ethyl-rapamycin, 40-O-[2-(N-methyl-N′-piperazinyl)acetoxy]ethylrapamycin, 39-O-desmethyl-39,40-O,O-ethylenerapamycin, (26R)-26-dihydro-40-O-(2-hydroxy)ethylrapamycin, 40-O-(2-aminoethyl)rapamycin, 40-O-(2-acetaminoethyl)rapamycin 40-O-(2-nicotinamidoethyl)rapamycin, 40-O-(2-(N-methyl-imidazo-2′-ylcarbethoxamido)ethyl)rapamycin, 40-O-(2-ethoxycarbonylaminoethyl)rapamycin, 40-O-(2-tolylsulfonamidoethyl)rapamycin, 40-0-[2-(4′,5′-dicarboethoxy-1′,2′,3′-triazol-1′-yl)-ethyl]-rapamycin, 42-epi-(tetrazolyl)rapamycin (tacrolimus), 42-[3-hydroxy-2-(hydroxymethyl)-2-methylpropanoate]rapamycin (temsirolimus), (42S)-42-deoxy-42-(1H-tetrazol-1-yl)rapamycin (zotarolimus), (3S,4R,5S,8R,9E,12S, 14S,15R,16S,18R,19R,26aS)-3-{(E)-2-[(1R,3R,4S)-4-Chlor-3-methoxycyclohexyl]-1-methylvinyl}-8-ethyl-5,6,8,11,12,13,14,15,16,17,18,19,24,25,26,26a-hexadecahydro-5,19.dihhydroxy-14,16-dimethoxy-4,10,12,18-tetramethyl-15,19-epoxy-3H-pyrido[2,1-c][1,4]oxazacyclotricosin-1,7,20,21(4H,23H)-tetron (pimecrolimus), (1R,2R,4S)-4-[(2R)-2-[(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl dimethylphosphinate (ridaforolimus).

The use of limus active agents, which may be in the form of microcrystals, such as the limus active agents rapamycin (sirolimus), everolimus, zotarolimus, umirolimus, deforolimus, myolimus, novolimus, pimecrolimus, ridaforolimus, tacrolimus, and temsirolimus is preferred.

The limus active agent is preferably selected from the group comprising or consisting of rapamycin (sirolimus), everolimus, zotarolimus, umirolimus, deforolimus, myolimus, novolimus, pimecrolimus, ridaforolimus, tacrolimus, and temsirolimus, further preferred are rapamycin (sirolimus) and everolimus. In an even more preferred embodiment, the limus active agent is rapamycin (sirolimus). In a particularly preferred embodiment, the limus active agent is everolimus.

Rapamycin is also known as Rapamun or the International Nonproprietary Name (INN) Sirolimus as well as the IUPAC name [3S-[3R*[E(1S*,3S*,4S*)],4S*,5R*,8S*, 9E,12R*,14R*,15S*,16R*,18S*,19S*,26aR*]]-5,6,8,11,12,13,14,15,16,17,18,19,24, 25,26,26a-hexadeca-hydro-5,19-dihydroxy-3-[2-(4-hydroxy-3-methoxy-cyclohexyl)-1-methylethenyl]-14,16-dimethoxy-4,10,12,18-tetramethyl-8-(2-propenyl)-15,19-epoxy-3H-pyrido[2,1-c][1,4]-oxaazacyclo-tricosine-1,7,20,21(4H,23H)-tetrone monohydrate. Rapamycin has the following structural formula:

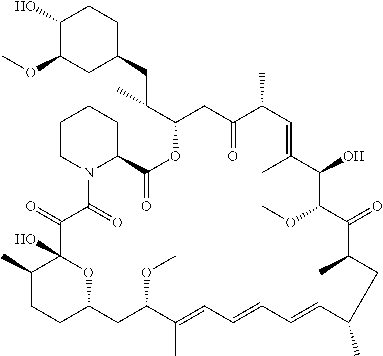 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

Everolimus is a derivative of rapamycin with the IUPAC name dihydroxy-12-[(2R)-1-[(1 S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.04,9]hexatriaconta-16,24,26,28-tetraen-2,3,10,14,20-penton.

Everolimus has the following structural formula:

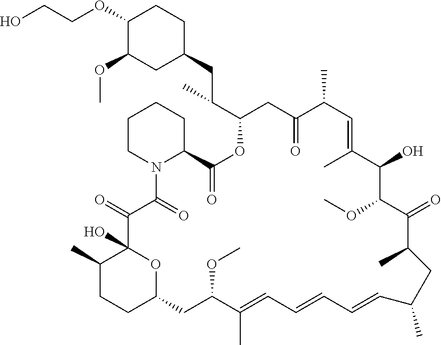 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

The term “microcrystals”, as used herein, refers to solids whose building blocks are regularly arranged in a crystal structure and have a size in the micrometer range. The term “micrometer range” as used herein corresponds to the range from 1 μm to 300 μm, where 1 μm corresponds to 10⁻⁶ m, 10⁻³ mm, or 1000 nm. Thus, the term microcrystals, as used herein, refers to crystals having a crystal size in the range of 1 μm to 300 μm.

The term “crystal size,” as used herein, refers to the length of the crystals along their largest dimension, i.e., along their longitudinal axis in the case of rod-shaped or needle-shaped crystals. Thus, the microcrystals, as defined herein, have a length in the range of 1 μm to 300 μm along their largest dimension.

The term “crystallinity,” as used herein, is the crystalline content of a compound, that is, the proportion of crystals of a compound to the total amount of that compound in crystalline and other forms.

The term “microcrystalline limus active agent”, as used herein, refers to a limus active agent that is in the form of microcrystals. Thus, the term “microcrystalline limus active agent” and “limus active agent in the form of microcrystals” are used interchangeably herein.

Crystallization processes for the preparation of limus active agents are known from the prior art. Generally, to crystallize limus active agents, a solution of a limus active agent can be prepared and the solubility of the limus active agent in the solution can be reduced. Common methods for reducing solubility include, for example, cooling, addition of an anti-solvent, and evaporation.

Crystallization by cooling: The limus active agent can be dissolved in a solvent at room temperature or higher temperature until saturation and brought to crystallization at lower temperature e.g. at 0° C. The crystal size distribution can be influenced by a controlled cooling rate. Both polar and non-polar organic solvents, such as toluene, acetonitrile, ethyl formate, isopropyl acetate, isobutyl acetate, ethanol, dimethyl formamide, anisole, ethyl acetate, methyl ethyl ketone, methyl isopropyl ketone, tetrahydrofuran, nitromethane, proprionitrile are suitable solvents for crystallization of limus active agents.

Crystallization by addition of seed crystals: The limus active agent is dissolved to saturation in a solvent and crystallization is initiated by the addition of seed crystals to achieve a controlled reduction of supersaturation.

Crystallization by addition of anti-solvent: The active agent is dissolved in a solvent and then a non-solvent or water is added. Two-phase mixtures are also possible here. Polar organic solvents such as acetone, acetonitrile, ethyl acetate, methanol, ethanol, isopropanol, butanol, butyl methyl ether, tetrahydrofuran, dimethyl formamide or dimethyl sulfoxide can be used as solvents for dissolving the limus active agent. Suitable non-solvents include pentane, hexane, cyclohexane or heptane. The solvent mixture can be allowed to stand for crystallization, stirred or slowly concentrated or evaporated in vacuo. The crystal size and crystallinity of the active agent can be influenced by controlled addition of the nonpolar solvent. Supersaturation should be slower to produce large crystals and faster to produce small crystals. Controlling the addition rate of the anti-solvent to control the crystal size is well known.

For the production of microcrystals, crystallization can also be assisted by ultrasound. It is generally known that crystal size can be influenced by means of ultrasound. Ultrasound can be used at the beginning of crystallization to initiate crystallization and nucleation, with further crystal growth then proceeding unhindered so that larger crystals can grow. On the other hand, the application of continuous ultrasonic sonication of a supersaturated solution leads to smaller crystals, as many nuclei are formed in this process, resulting in the growth of numerous small crystals. Another option is to use ultrasound in pulse mode to influence crystal growth in such a way that tailored crystal sizes are achieved.

Herein, preferred crystallization processes for the production of microcrystalline limus active agents are controlled crystallization to obtain microcrystals in native and intact state and to avoid possible damage, e.g. by milling or micronization.

Other processes known from the prior art, such as micronization, grinding or sieving, can also be used to provide the desired crystal sizes. One possibility is to grind the crystals, which can also be done during crystallization by wet grinding. Milling can be advantageous to obtain different crystal sizes i.e. a broader crystal size distribution. Milling allows for all desired sizes in the crystal size range. More uniform crystal sizes can be provided by performing, for example, a special sieving process after isolation and drying. Special sieving devices known from the prior art can be used for this purpose. In the sieving process, the limus active agent crystals can be sieved through a stack of sieves, for example, and divided into different size ranges.

Example images of the limus active agents rapamycin and everolimus in the form of microcrystals are shown in FIG. 2 to FIG. 9. FIGS. 2 and 3 show rapamycin in rod form with very narrow particle size distribution in the range of 10 μm to 30 μm. FIGS. 4 and 5 show rapamycin in the form of microcrystals in rod form with extremely narrow particle size distribution ranging from 15 μm to 30 μm. FIGS. 7 and 8 show rapamycin with particle size distribution ranging from 20 μm to 40 μm. FIGS. 6 and 7 show everolimus in needle form with a particle size distribution in the range of 20 μm to 40 μm. In FIGS. 2 to 9, it is clear that no larger crystals or agglomerates are present. It is also clear that everolimus is in the form of needles, while rapamycin is in the form of rhombohedral prisms.

It could be shown that particularly stable suspensions can be prepared with microcrystalline limus active agents if the suspensions contain at least one tri-O-acylglycerol selected from the group consisting of trioctanoylglycerol, trinonanoylglycerol, tridecanoylglycerol, and triundecanoylglycerol. According to the invention, the at least one limus active agent is in the form of microcrystals. Thus, the limus active agent is present in the form of microcrystals having a crystal size in the range of 1 μm to 300 μm. The microcrystals of the at least one limus active agent therefore have a crystal size between 1 μm to 300 μm.

The microcrystals of the limus active agent are not encapsulated and are not coated, e.g. with a polymer and are not modified on the surface. In addition, the microcrystals of limus active agent do not contain a polymer, polymer particles, metal, metal particles, ceramic or ceramic particles. Nor do they contain any other active pharmaceutical inactive agent or proteins, amino acid or nucleotides or other biopolymers.

The present invention therefore relates to a suspension comprising at least one microcrystalline limus active agent as defined herein. In the suspension, the at least one limus active agent is present in the form of microcrystals. The content of limus active agent dissolved in the solvent or solvent mixture in the suspension is less than 10%, preferably less than 5% and further preferably less than 2%, most preferably less than 1% based on the mass of limus active agent in the form of microcrystals used in the preparation of the suspension. It is therefore preferred that a maximum of 10%, preferably a maximum of 5% and further preferably a maximum of 2%, most preferably a maximum of 1% of the microcrystals of the at least one limus active agent dissolve in the suspension.

According to the invention, the suspension for coating of a medical device, preferably selected from a catheter balloon, a balloon catheter, a stent, or a cannula, contains a solvent or a solvent mixture in which the at least one tri-O-acylglycerol dissolves and in which the microcrystals of the at least one limus active agent do not dissolve.

The phrase “in which the microcrystals of the at least one limus active agent do not dissolve”, as used herein, means that preferably a maximum of 10%, preferably a maximum of 9%, further preferably a maximum of 8%, further preferably a maximum of 7%, further preferably a maximum of 6%, further preferably a maximum of 5%, still more preferably a maximum of 3%, further preferably a maximum of 2%, and most preferably a maximum of 1% of the microcrystals of the at least one limus active agent dissolve in the suspension. Preferably, of course, 100% of the microcrystals of the at least one limus active agent do not dissolve in the suspension.

It is further preferred that the solubility of the microcrystals of the at least one limus active agent in the solvent or solvent mixture of the suspension is <20 mg/mL, more preferably <15 mg/mL, more preferably <10 mg/mL, more preferably <9 mg/mL, more preferably <8 mg/mL, further preferably <7 mg/mL, further preferably <6 mg/mL, further preferably <5 mg/mL, further preferably <4 mg/mL, further preferably <3 mg/mL, further preferably <2 mg/mL, further preferably <1 mg/mL.

Surprisingly, it was found that microcrystals of limus active agents do not dissolve in a solution containing at least one tri-O-acylglycerol selected from the group consisting of trioctanoylglycerol, trinonanoylglycerol, tridecanoylglycerol, and triundecanoylglycerol. Thus, a crystal suspension can be prepared as a coating formulation in which the microcrystals of the limus active agent remain intact.

The crystals of the limus active agent should have a size of at least 1 μm. Crystals of less than 1 μm are too small, so they dissolve relatively quickly. In preparing the suspension of the present invention, it has been found that stable suspensions can be obtained only when the at least one limus active agent has substantially no crystals with a crystal size of less than 1 μm. In other words, the limus active agent is particularly preferably not in the form of nanocrystals. The term nanocrystals, as used herein, refers to crystals having a crystal size in the range of 1 nm to less than 1000 nm.

Preferably, at least 95%-97% of the at least one limus active agent, more preferably at least 95%-99%, further preferably at least 97%-99%, and particularly preferably at least 98%-99.9% of the at least one limus active agent is present in the form of microcrystals having a crystal size of at least 1 μm. In further preferred embodiments, 100% of the at least one limus active agent is present in the form of microcrystals with a crystal size of at least 1 μm.

It could also be shown that it is advantageous if the limus active agent in the form of microcrystals has a crystal size of at least 10 μm. Therefore, it is preferred that the at least one limus active agent has a small proportion of microcrystals with a crystal size of 1 μm-10 μm. It is particularly preferred that only a few crystals, i.e. significantly less than 10% of all crystals are smaller than 10 μm. In preferred embodiments, less than 10% of all microcrystals of the limus active agent are present with a crystal size in the range of less than 10 μm.

Preferably, at least 90% of the at least one limus active agent, preferably at least 90%-95% of the at least one limus active agent, more preferably at least 93%-98% of the at least one limus active agent, more preferably at least 95%-99% of the at least one limus active agent, and particularly preferably at least 98%-99.9% of the at least one limus active agent is present in the form of microcrystals having a crystal size of at least 10 μm.

It is further preferred that the microcrystals of the at least one limus active agent have a crystal size of at least 5 μm. It is therefore preferred that at least 90% of the at least one limus active agent, preferably at least 90%-95% of the at least one limus active agent, more preferably at least 93%-98% of the at least one limus active agent, further preferably at least 95%-99% of the at least one limus active agent, and particularly preferably at least 98%-99.9% of the at least one limus active agent is present in the form of microcrystals with a crystal size of at least 5 μm. Microcrystals with a crystal size in the range of smaller than 5 μm can dissolve faster and are therefore less preferred.

Still further preferred is that the microcrystals of the at least one limus active agent have a crystal size of at least 20 μm. It is therefore preferred that at least 90% of the at least one limus active agent, preferably at least 90%-95% of the at least one limus active agent, more preferably at least 93%-98% of the at least one limus active agent, further preferably at least 95%-99% of the at least one limus active agent and particularly preferably at least 98%-99.9% of the at least one limus active agent is present in the form of microcrystals having a crystal size of at least 20 μm.

In addition, preferably only a few microcrystals of the limus active agent, i.e. less than 40% and more preferably less than 30% or even less than 25%, are present with a crystal size in the range of 50 μm-300 μm. It is therefore preferred that a maximum of 40% of the at least one limus active agent, preferably a maximum of 30% of the at least one limus active agent, further preferably a maximum of 25% of the at least one limus active agent is present in the form of microcrystals with a particle size of 50 μm-300 μm. In further embodiments, it is preferred that a maximum of 20% of the at least one limus active agent, preferably a maximum of 15% of the at least one limus active agent, further preferably a maximum of 10% of the at least one limus active agent is present in the form of microcrystals having a particle size of 50 μm-300 μm. In particularly preferred embodiments, the microcrystals of the at least one limus active agent are substantially present with a crystal size of at most 50 μm.

Further preferably, very few microcrystals of the limus active agent are present, i.e. less than 10% and more preferably less than 5% or even less than 2%, and most preferably less than 1% with a crystal size in the range of 100 μm-300 μm. Microcrystals with a crystal size in the range of 100 μm-300 μm could form agglomerates and coalesce into larger particles, which may pose the risk of vascular occlusion. It is therefore particularly preferred if the proportion of microcrystals with a particle size in the range 100 μm-300 μm is as low as possible.

It is therefore preferred that at most 10% of the at least one limus active agent, preferably at most 5% of the at least one limus active agent, further preferably at most 2% of the at least one limus active agent, still more preferably at most 1% of the at least one limus active agent is present in the form of microcrystals having a particle size of 100 μm-300 μm. In further embodiments, it is preferred that at least 99%, preferably 99.5%, further preferably at least 99.7%, still further preferably at least 99.9% and most preferably 100% of the at least one limus active agent is present with a particle size of <100 μm. In preferred embodiments, the microcrystals of the at least one limus active agent are substantially present with a crystal size of at most 100 μm. In particularly preferred embodiments, the microcrystals of the at least one limus active agent are substantially present with a crystal size of at most 100 μm.

Preferably, the limus active agent is in the form of microcrystals with a crystal size of 1 μm to 100 μm.

It could be shown that microcrystals of the limus active agent with a crystal size in the range of 10 μm to 50 μm are well suited for providing a suspension according to the invention for coating of medical devices. It is therefore preferred that at least 70% of the at least one limus active agent, preferably at least 70%-80% of the at least one limus active agent, further preferably at least 80%-90% of the at least one limus active agent, further preferably at least 90%-95% of the at least one limus active agent, and particularly preferably at least 95%-99% of the at least one limus active agent is present in the form of microcrystals with a crystal size of 10 μm to 50 μm.

It could be shown that microcrystals of the limus active agent with a particle size in the range of 5 μm to 35 μm are suitable for providing a stable suspension for coating medical devices. It is therefore preferred that at least 70% of the microcrystals of the limus active agent are present with a crystal size in the range of 5 μm to 35 μm. It is therefore preferred that at least 70% of the limus active agent, preferably at least 70%-80% of the limus active agent, further preferably at least 80%-90% of the limus active agent is present in the form of microcrystals with a particle size ranging from 5 μm to 35 μm. It is further preferred that at least 70% of the at least one limus active agent, preferably at least 70%-80% of the at least one limus active agent, further preferably at least 80%-90% of the at least one limus active agent, further preferably at least 90%-95% of the at least one limus active agent, and particularly preferably at least 95%-99% of the at least one limus active agent is present in the form of microcrystals having a particle size of 5 μm to 35 μm.

In particularly preferred embodiments, the microcrystals of the limus active agent are present with a crystal size in the range of 20 μm to 40 μm. Preferably, therefore, at least 70% of the at least one limus active agent, preferably at least 70%-80% of the at least one limus active agent, further preferably at least 80%-90% of the at least one limus active agent, further preferably at least 90%-95% of the at least one limus active agent, and particularly preferably at least 95%-99% of the at least one limus active agent is present in the form of microcrystals having a crystal size ranging from 20 μm to 40 μm.

Preferably, the limus active agent has a crystallinity of at least 90% by weight, more preferably at least 92.5% by weight, more preferably at least 95% by weight, more preferably at least 97.5% by weight and most preferably at least 99% by weight.

The microcrystals of the at least one limus active agent are preferably microcrystals of at least one limus active agent selected from the group comprising or consisting of rapamycin, everolimus, zotarolimus, umirolimus, deforolimus, myolimus, novolimus, pimecrolimus, ridaforolimus, tacrolimus, and temsirolimus.

The microcrystals of the at least one limus active agent are preferably microcrystals of rapamycin or microcrystals of everolimus. Preferred herein are microcrystalline rapamycin and microcrystalline everolimus. Particularly preferred herein is microcrystalline everolimus.

Crystals with prismatic to acicular habit are one-dimensional elongated forms in which the length of the crystal is significantly greater than its diameter.

In preferred embodiments, the at least one limus active agent is rapamycin. Rapamycin crystallizes in the form of rhombohedral prisms. It is therefore preferred that at least 90%, more preferably at least 92.5%, more preferably at least 95%, more preferably at least 97.5%, and most preferably at least 99% of the microcrystals of rapamycin are in the form of rhombohedral prisms. It is therefore preferred that at least 90%, more preferably at least 92.5%, more preferably at least 95%, more preferably at least 97.5% and most preferably at least 99% of the microcrystals of rapamycin are prismatic. It is therefore preferred that at least 90%, more preferably at least 92.5%, more preferably at least 95%, more preferably at least 97.5% and most preferably at least 99% of rapamycin is in the form of prismatic microcrystals.

In preferred embodiments, the at least one limus active agent is everolimus. Everolimus crystallizes in the form of needles. It is therefore preferred that at least 90%, more preferably at least 92.5%, more preferably at least 95%, more preferably at least 97.5%, and most preferably at least 99% of the microcrystals of everolimus are in the form of needles. It is therefore preferred that at least 90%, more preferably at least 92.5%, more preferably at least 95%, more preferably at least 97.5% and most preferably at least 99% of the microcrystals of everolimus are needle-shaped. It is therefore preferred that at least 90%, more preferably at least 92.5%, more preferably at least 95%, more preferably at least 97.5%, and most preferably at least 99% of everolimus is in the form of needle-shaped microcrystals.

The term “solvent”, as used herein, refers to a substance that exists in the liquid state of aggregation at normal temperature (20° C.) and normal pressure (101 hPa; 1 bar, 1 atm) and that can dissolve or dilute gases, liquids or solids without chemical reactions taking place between the dissolved substance and the solvent. Liquids such as water and liquid organic substances are used as solvents to dissolve other substances.

The term “non-solvent,” as used herein, refers to a solvent that cannot dissolve or dissolve microcrystalline limus active agents, i.e. a solvent in which microcrystalline limus active agents are virtually insoluble, but in which the tri-O-acylglycerols selected from the group consisting of trioctanoylglycerol, trinonanoylglycerol, tridecanoylglycerol, and triundecanoylglycerol, or mixtures of said tri-O-acylglycerols, are soluble.

The solubility of a limus microcrystalline active agent in a non-solvent should be at most 1 mg/mL. Examples of solvents in which the solubility of microcrystalline limus active agents is at most 1 mg/mL are water and some nonpolar organic solvents such as saturated aliphatic hydrocarbons.

Examples of solvents in which the tri-O-acylglycerols trioctanoylglycerol, trinonanoylglycerol, tridecanoylglycerol, and triundecanoylglycerol or mixtures of said tri-O-acylglycerols are soluble include, but are not limited to non-polar organic solvents such as hexane, heptane, cyclohexane, toluene, but also polar organic solvents such as diethyl ether, ethyl acetate, acetone, isopropanol and ethanol.

Tri-O-acylglycerols are nonpolar, i.e. lipophilic, and are sparingly or virtually insoluble in very polar solvents such as water or glycerol. A suspension containing as solvent exclusively a very polar solvent such as water or glycerol is thus not according to the invention, since the tri-O-acylglycerols selected from the group consisting of trioctanoylglycerol, trinonanoylglycerol, tridecanoylglycerol, and triundecanoylglycerol or a mixture of said tri-O-acylglycerols do not exist in dissolved form in very polar solvents such as water or glycerol.

The term “non-solvent”, as used herein, thus refers to nonpolar organic solvents, particularly saturated aliphatic hydrocarbons. A “non-solvent” may therefore also be referred to as a “nonpolar organic non-solvent.” Non-polar organic solvents referred to herein as “non-solvent” therefore include saturated aliphatic hydrocarbons that are liquid at normal temperature (20° C.) and normal pressure (101 hPa; 1 bar, 1 atm), i.e. unbranched (linear) saturated hydrocarbons with the general molecular formula Cₙ H₂ₙ₊₂ with n=5 to 16, branched saturated hydrocarbons with the general molecular formula Cₙ H₂ₙ₊₂ with n=4 to 16, or cyclic saturated hydrocarbons with the general molecular formula Cₙ H₂ₙ with n=5 to 16. Examples of non-solvers include, but are not limited to, unbranched C₅₋₁₆ alkanes such as pentane, hexane, heptane, octane, nonane, decane, petroleum ether, branched C₅₋₁₆-alkanes (iso-alkanes) such as isopentane, isooctane, 2-methylpentane, 3-methylpentane, 2,2-dimethylbutane, 2,3-dimethylbutane, 2,2-dimethylpentane, 2-methylhexane, 3-methylhexane, 2,3-dimethylpentane, 2,4-dimethylpentane, 3,3-dimethylpentane, 3-ethylpentane, 2,2,4-trimethylpentane, 2,2,4-trimethylbutane, 2-methyloctane, 2-methylheptane, 3-methylheptane, 4-methylheptane, tetraethylmethane, C₅₋₁₆-cycloalkanes such as cyclopentane, cyclohexane, methylcyclopentane, tert-butylcyclohexane, methylcyclohexane, cycloheptane, cyclooctane, cyclononane, cyclodecane, 2,3-dimethylcyclobutane, 1,2-dimethylcyclobutane, decalin, pinane hexylcyclohexane, heptylcyclopentane, 1,4-dimethylcyclohexane, 1,1-dimethylcyclohexane, spiropentane, spirohexane, spiroheptane. Of course, mixtures of non-solvents can also be used.

Non-solvents suitable for the present invention are in the liquid state at normal temperature (20° C.) and normal pressure (101 hPa; 1 bar, 1 atm). Preferred non-solvents have a melting point of <20° C., more preferably <15° C., even more preferably <10° C. Preferred non-solvents also exhibit a boiling point of <200° C., further preferably <150° C., even more preferably <100° C. Preferred non-solvents also exhibit a boiling point of >25° C., further preferably >30° C., even more preferably of >40° C. Preferred non-solvents therefore have a boiling point between 25° C. and 200° C., further preferably between 30° C. and 150° C., still more preferably between 40° C. and 100° C. The indications on melting points and boiling points refer here to normal pressure (101 hPa; 1 bar, 1 atm).

Preferred non-solvents also have a vapor pressure at normal temperature (20° C.) of <600 hPa, more preferably <300 hPa, even more preferably <200 hPa. Preferred non-solvents further exhibit a vapor pressure at 20° C. of >1 hPa, more preferably >10 hPa, even more preferably >30 hPa. Preferred non-solvents therefore exhibit a vapor pressure normal temperature (20° C.) of between 1 hPa to 600 hPa, more preferably between 10 hPa and 300 hPa, still more preferably between 30 hPa and 200 hPa.

Preferred non-solvents have no permanent dipole moment, i.e., have a dipole moment of 0.0 to a maximum of 0.1 D (0.0-0.3·10⁻³⁰ Cm).

Preferred non-solvents exhibit a dielectric constant εᵣ of ≤2.5, more preferably ≤2.2, even more preferably ≤2.0 at normal temperature (20° C.). Preferred non-solvents exhibit an n-octanol-water partition coefficient log K_{OW} of >2.0, more preferably of ≥2.5, even more preferably of ≥3.0. Preferred non-solvents are therefore those that have a dielectric constant εᵣ of ≤2.5 and a log K_{OW} of >2.0 at normal temperature (20° C.), more preferably a dielectric constant εᵣ of ≤2.2 and a log K_{OW} of ≥2.5, still more preferably a dielectric constant εᵣ of ≤2.0 and a log K_{OW} of ≥3.0.

Preferred non-solvents also have a density at normal temperature (20° C.) of <0.95 g/mL, more preferably <0.9 g/mL, even more preferably <0.8 g/mL. Preferred non-solvents also have a viscosity at normal temperature (20° C.) of <2.0 mPa s, more preferably <1.5 mPa s, even more preferably <1.0 mPa s.

Thus, preferred herein are non-solvents that are in the liquid state of aggregation at normal temperature (20° C.) and normal pressure (101 hPa; 1 bar, 1 atm) and have a melting point of <20° C., more preferably <15° C., still more preferably <10° C., a boiling point of <200° C., more preferably <150° C., still more preferably of <100° C., resp. a boiling point of >25° C., more preferably >30° C., even more preferably of >40° C., a vapor pressure at 20° C. of <600 hPa, more preferably <300 hPa, even more preferably <200 hPa, respectively. a vapor pressure at 20° C. of >1 hPa, more preferably >10 hPa, still more preferably >30 hPa, a density of <0.95 g/mL, more preferably <0.9 g/mL, still more preferably <0.8 g/mL, a dipole moment of 0.0-0.1 D (0.0-0.3·10⁻³⁰ Cm), a viscosity at 20° C. of <2.0 mPa s, still more preferably <1.5 mPa s, even more preferably <1.0 mPa s, and in particular have a dielectric constant εᵣ at 20° C. of ≤2.5, more preferably ≤2.2, even more preferably ≤2.0, an n-octanol-water partition coefficient log K_{OW} of >2.0, more preferably ≥2.5, even more preferably 3.0.

An overview of orientation values (the values given are rounded values) of the dielectric constant and log K_{OW} of non-solvents suitable herein is shown in table 1. An overview of other parameters of some specific non-solvents is shown in table 2 (the values given are rounded values).

TABLE 1
Overview of dielectric constants and
logK_{OW} values of non-solvents (rounded values)

| Solvent | Dielectric constant ∈ᵣ (20° C.) | logK_{OW} |
|---|---|---|
| n-alkanes | approx. 1.80-2.00 | approx. 3-8 |
| iso-alkanes | approx. 1.80-1.95 | approx. 3-8 |
| cycloalkanes | approx. 1.90-2.20 | approx. 3-8 |

TABLE 2
Overview of some physical parameters of some non-solvents (rounded values).

| | Dielectric | Boiling | Melting | Vapor | |
|---|---|---|---|---|---|
| | constant ∈ᵣ | point | point | pressure | |
| Solvent | (20° C.) | (° C.) | (° C.) | (20° C., hPa) | logK_{OW} |
| Pentane | 1.84 | 36.1 | −129.7 | 573 | 3.2 |
| Hexane | 1.89 | 69 | −95 | 160 | 3.8 |
| Heptane | 1.92 | 98 | −90.6 | 48 | 4.5 |
| Octane | 1.95 | 126 | −57 | 14 | 5.2 |
| Nonan | 1.97 | 151 | −54 | 4.8 | 5.7 |
| Decan | 2.00 | 174 | −30 | 1.66 | 6.3 |
| Undecan | 2.01 | 196 | −26 | 0.55 | 6.5 |
| Isopentane | 1.85 | 28 | −160 | 761 | 3.2 |
| 2-Methylpentane | 1.89 | 60 | −154 | 227 | 3.6 |
| 2,2-Dimethylbutane | 1.87 | 50 | −100 | 348 | 3.8 |
| 2,2-Dimethylpentane | 1.91 | 79 | −123 | 111 | 3.8 |
| 2-Methylhexane | 1.92 | 90 | −118 | 53 | 3.7 |
| 2,3-Dimethylpentane | 1.94 | 90 | −135 | 72 | 3.8 |
| 2,2,4-Trimethylpentane | 1.94 | 98 | −107 | 53 | 4.1 |
| Cyclopentane | 1.97 | 49.3 | −93.9 | 346 | 3.0 |
| Cyclohexane | 2.02 | 80.7 | 6.5 | 104 | 3.4 |
| Methylcyclopentane | 1.99 | 71 | −143 | 18 | 3.4 |
| tert-butylcyclohexane | 2.08 | 167 | −41 | <20 | 4.0 |
| Methylcyclohexane | 2.02 | 101 | −127 | <20 | 3.9 |
| Cycloheptane | 2.08 | 119 | −8 | 22.3 | 4 |
| Cyclooctane | 2.12 | 150 | 13 | 5.5 | 4.5 |
| Cyclononan | 2.14 | 170 | 11 | <5 | 5.1 |
| Cyclodecane | 2.15 | 201 | 10 | <5 | 5.5 |

Preferred non-solvents with a melting point of <15° C., a dielectric constant εᵣ at 20° C. of <2.5, a log K_{OW} of ≥3.0, a boiling point of <200° C., a boiling point of >30° C., a vapor pressure at 20° C. between 1 hPa and 600 hPa include but are not limited to pentane, hexane, heptane, octane, nonane, decane, petroleum ether, isooctane, 2-methylpentane, 3-methylpentane, 2,2-dimethylbutane, 2,3-dimethylbutane, 2,2-dimethylpentane, 2-methylhexane, 3-methylhexane, 2,3-dimethylpentane, 2,4-dimethylpentane, 3,3-dimethylpentane, 3-ethylpentane, 2,2,4-trimethylpentane, 2,2,4-trimethylbutane, 2-methyloctane, 2-methylheptane, 3-methylheptane, 4-methylheptane, tetraethylmethane, cyclopentane, cyclohexane, methylcyclopentane, tert-butylcyclohexane, methylcyclohexane, cycloheptane, cyclooctane, cyclononane, cyclodecane, 2,3-dimethylcyclobutane, 1,2-dimethylcyclobutane, decalin, pinane. The melting points and boiling points given here refer to normal pressure (101 hPa; 1 bar, 1 atm).

Preferred non-solvents herein include pentane, cyclopentane, hexane, cyclohexane, heptane, octane, nonane and decane.

Further preferred nonsolvents having a melting point of <10° C., a dielectric constant εᵣ at 20° C. of ≤2.0, a log K_{OW} of ≥3.0, a boiling point of <150° C., a boiling point of >30° C., a vapor pressure at 20° C. between 10 hPa and 600 hPa include but are not limited to pentane, hexane, heptane, octane, petroleum ether, isooctane, 2-methylpentane, 3-methylpentane, 2,2-dimethylbutane, 2,3-dimethylbutane, 2,2 dimethylpentane, 2-methylhexane, 3-methylhexane, 2,3-dimethylpentane, 2,4-dimethylpentane, 3,3-dimethylpentane, 3-ethylpentane, 2,2,4-trimethylpentane, 2,2,4-trimethylbutane, 2-methyloctane, 2-methylheptane, 3-methylheptane, 4-methylheptane, tetraethylmethane, cyclopentane, cyclohexane, methylcyclopentane, methylcyclohexane, cycloheptane, 2,3-dimethylcyclobutane, 1,2-dimethylcyclobutane. The melting points and boiling points given here refer to normal pressure (101 hPa; 1 bar, 1 atm).

Still further preferred non-solvents having a melting point of <10° C., a dielectric constant εᵣ at 20° C. of <2.0, a log K_{OW} of ≥3.0, a boiling point of <100° C., a boiling point of >40° C., a vapor pressure at 20° C. between 30 hPa and 300 hPa include but are not limited to hexane, heptane, 2-methylpentane, 3-methylpentane, 2,2-dimethylpentane, 2-methylhexane, 3-methylhexane, 2,3-dimethylpentane, 2,4-dimethylpentane, 3,3-dimethylpentane, 3-ethylpentane, 2,2,4-trimethylpentane, 2,2,4-trimethylbutane, cyclohexane, cycloheptane, 2,3-dimethylcyclobutane, 1,2-dimethylcyclobutane. The melting points and boiling points given here refer to normal pressure (101 hPa; 1 bar, 1 atm).

Herein, particularly preferred non-solvents are hexane, cyclohexane and heptane.

The term “non-polar organic solvent”, as used herein, refers to a carbon-based solvent that is liquid at normal temperature (20° C.) and pressure (101 hPa; 1 bar, 1 atm), i.e., has at least a melting point of <20° C. Examples of non-polar organic solvents include, but are not limited to, carbon tetrachloride, pure hydrocarbon solvents such as, for example, pentane, cyclopentane, hexane, cyclohexane, heptane, octane, nonane or decane, aromatic solvents such as toluene, benzene, xylene.

Non-polar organic solvents, as defined herein, have a dielectric constant εᵣ at 20° C. of ≤10, more preferably of ≤5.0, more preferably ≤3.0, even more preferably of ≤2.0 and simultaneously an n-octanol-water partition coefficient log K_{OW} >2.0, more preferably of ≥2.5, even more preferably of ≥3.0. Thus, a solvent with a dielectric constant εᵣ at 20° C. of ≤10 and a log K_{OW} ≤2.0, particularly ≤1.5 does not constitute a nonpolar organic solvent herein. For example, 1,4-dioxane has a dielectric constant εᵣ at 20° C. of about 2.3, but a log K_{OW} of about −0.4, and thus does not represent a nonpolar organic solvent herein.

Preferred are nonpolar organic solvents that have a dielectric constant εᵣ of ≤10 at normal temperature (20° C.) and a log K_{OW} of >2.0, more preferably a dielectric constant εᵣ of ≤5 and a log K_{OW} of ≥2.5, still more preferably a dielectric constant εᵣ of ≤3.0 and a log K_{OW} of ≥3.0. In particular, nonpolar organic solvents that have a dielectric constant εᵣ of ≤2.0 and a log K_{OW} of >3.0 at normal temperature (20° C.) are preferred.

Non-polar organic solvents suitable for the present invention exist in the liquid state of aggregation at normal temperature (20° C.) and normal pressure (101 hPa; 1 bar, 1 atm). Preferred nonpolar organic solvents have a melting point of <20° C., more preferably <15° C., still more preferably <10° C. Preferred non-polar organic solvents also exhibit a boiling point of <200° C., further preferably <150° C., even more preferably <100° C. Preferred nonpolar organic solvents also exhibit a boiling point of >25° C., more preferably >30° C., even more preferably of >40° C. Preferred nonpolar organic solvents therefore have a boiling point of between 25° C. and 200° C., further preferably between 30° C. and 150° C., still more preferably between 40° C. and 100° C. The indications on melting points and boiling points refer here to normal pressure (101 hPa; 1 bar, 1 atm).

Preferred non-polar organic solvents also exhibit a vapor pressure at normal temperature (20° C.) of <600 hPa, more preferably <300 hPa, even more preferably <200 hPa. Preferred non-polar organic solvents further exhibit a vapor pressure at 20° C. of >1 hPa, more preferably >10 hPa, even more preferably >30 hPa. Preferred non-polar organic solvents therefore exhibit a vapor pressure normal temperature (20° C.) of between 1 hPa to 600 hPa, more preferably between 10 hPa and 300 hPa, still more preferably between 30 hPa and 200 hPa.

Examples of nonpolar organic solvents that have a dielectric constant εᵣ of <10 at normal temperature (20° C.) and a log K_{OW} of >2 include, but are not limited to, unbranched C₅₋₁₆-alkanes such as pentane, hexane, heptane, octane, nonane, decane, petroleum ether, branched C₅₋₁₆-alkanes such as isopentane, isooctane, 2 methylpentane, 3-methylpentane, 2,2-dimethylbutane, 2,3-dimethylbutane, 2,2-dimethylpentane, 2-methylhexane, 3-methylhexane, 2,3-dimethylpentane, 2,4-dimethylpentane, 3,3-dimethylpentane, 3-ethylpentane, 2,2,4-trimethylpentane, 2,2,4-trimethylbutane, 2-methyloctane, 2-methylheptane, 3-methylheptane, 4-methylheptane, tetraethylmethane, or C₅₋₁₆-cycloalkanes such as cyclopentane, cyclohexane, methylcyclopentane, tert-butylcyclohexane, methylcyclohexane, cycloheptane, cyclooctane, cyclononane, cyclodecane, 2,3-dimethylcyclobutane, 1,2-dimethylcyclobutane, decalin, pinane 1,4-dimethylcyclohexane, 1,1-dimethylcyclohexane, spiropentane, spirohexane, spiroheptane, ligroin, haloalkanes such as carbon tetrachloride, tetradecafluorohexane, aromatic hydrocarbons such as benzene, aromatic hydrocarbons with saturated aliphatic substituents such as toluene, o-xylene, m-xylene, p-xylene, mesitylene, 1-phenylbutane, 2-methyl-1-phenylpropane, 2-phenbutane, cumene, iso-butylbenzene, propylbenzene, hexylbenzene, iso-butylbenzene, halogenated aromatics such as chlorobenzene, fluorobenzene, p-dichlorobenzene, o-dichlorobenzene, hexafluorobenzene, bromobenzene, benzyl chloride, benzyl bromide or other substituted aromatics such as anisole and ethoxybenzene.

Thus, nonpolar organic solvents are preferred herein, which are in the liquid state of aggregation at normal temperature (20° C.) and normal pressure (101 hPa; 1 bar, 1 atm) and have a melting point of <20° C., more preferably <15° C., even more preferably <10° C., a boiling point of <200° C., more preferably <150° C., even more preferably <100° C., resp. a boiling point of >25° C., more preferably >30° C., even more preferably of >40° C., a vapor pressure at 20° C. of <600 hPa, more preferably <300 hPa, even more preferably <200 hPa, respectively a vapor pressure at 20° C. of >1 hPa, more preferably >10 hPa, still more preferably >30 hPa, a dipole moment of 0.0-0.5 D (0.0-1.7 10⁻³⁰ Cm), preferably 0.0-0.1 D (0.0-0.3 10⁻³⁰ Cm), and more preferably a dielectric constant εᵣ at 20° C. of ≤10, more preferably ≤5.0, even more preferably of ≤3.0, even more preferably of ≤2.0, and an n-octanol-water partition coefficient log K_{OW} of >2.0, more preferably of ≥2.5, even more preferably of ≥3.0.

The term “nonpolar organic solvent” as used herein preferably refers to aprotic nonpolar solvents, which are nonpolar due to the small differences in electronegativity between carbon and hydrogen and have no permanent dipole moment, less preferred are therefore halogenated aromatics such as chlorobenzene, fluorobenzene, p-dichlorobenzene, o-dichlorobenzene, bromobenzene, benzyl chloride, benzyl bromide or further substituted aromatics such as anisole, ethoxybenzene, which have a dipole moment of at least 1.0 D (3.3*10⁻³⁰ Cm) or a dielectric constant εᵣ at 20° C. of ≥3.

Preferred nonpolar organic solvents therefore have a dipole moment of 0.0-0.5 D (0.0-1.7·10⁻³⁰ Cm), and further preferred nonpolar organic solvents have no permanent dipole moment, i.e. have a dipole moment of 0.0-0.1 D (0.0-0.3=10⁻³⁰ Cm).

Aprotic nonpolar solvents are very lipophilic and very hydrophobic and are therefore preferred herein as nonpolar organic solvents. Representatives of aprotic nonpolar solvents preferred herein are alkanes, benzene and aromatics with aliphatic and aromatic substituents, perhalogenated hydrocarbons, e.g. carbon tetrachloride, hexafluorobenzene.

Other representatives of aprotic-nonpolar solvents are alkenes, alkynes, aromatics with unsaturated aliphatic substituents and other molecules with a completely symmetrical structure, such as tetramethylsilane or carbon disulfide. The aprotic nonpolar solvents such as the alkenes, alkynes, aromatics with unsaturated aliphatic substituents, and other molecules of completely symmetrical construction, such as tetramethylsilane or carbon disulfide, can be used herein as nonpolar organic solvents, but are less preferred. If such aprotic nonpolar solvents are used, it must be ensured that no chemical reactions occur between them and the microcrystalline limus active agent and the at least one tri-O-acylglycerol. A person skilled in the art is readily able to assess whether chemical reactions can occur between a particular solvent and a microcrystalline limus active agent or tri-O-acylglycerol, and whether a particular solvent is suitable for preparing a crystal suspension. The selection of a suitable solvent is therefore part of the routine work of a person skilled in the art.

Preferred non-polar organic solvents herein are therefore unbranched, branched and cyclic saturated aliphatic hydrocarbons, aromatic hydrocarbons and aromatic hydrocarbons with saturated aliphatic substituents and perhalogenated hydrocarbons.

Preferred non-polar organic solvents thus exhibit a dielectric constant εᵣ of ≤3, more preferably of ≤2.5, more preferably ≤2.2, even more preferably of ≤2.0 and an n-octanol-water partition coefficient log K_{OW} >2.0, more preferably of ≥2.5, even more preferably of ≥3.0 at normal temperature (20° C.) and normal pressure (101 hPa; 1 bar, 1 atm).

Thus, particularly preferred herein are nonpolar organic solvents that are in the liquid state of aggregation at normal temperature (20° C.) and normal pressure (101 hPa; 1 bar, 1 atm) and have a melting point of <20° C., more preferably <15° C., still more preferably <10° C., a boiling point of <200° C., more preferably <150° C., still more preferably <100° C., respectively a boiling point of >25° C., more preferably >30° C., even more preferably of >40° C., a vapor pressure at 20° C. of <600 hPa, more preferably <300 hPa, even more preferably <200 hPa, respectively. a vapor pressure at 20° C. of >1 hPa, more preferably >10 hPa, still more preferably >30 hPa, a dipole moment of 0.0-0.5 D (0.0-1.7·10⁻³⁰ Cm), preferably 0.0-0.1 D (0.0-0.3·10⁻³⁰ Cm), and a dielectric constant εᵣ at 20° C. of ≤3, more preferably ≤2.5, even more preferably of ≤2.2, even more preferably of ≤2.0, and an n-octanol-water partition coefficient log K_{OW} of >2.0, more preferably of ≥2.5, even more preferably of ≥3.0.

An overview of orientation values (the values given are rounded values) of the dielectric constants and log K_{OW} of nonpolar organic solvents is shown in Table 3.

TABLE 3
Overview of dielectric constants and logK_{OW} values
for nonpolar organic solvents (rounded values).

| | Dielectric | |
|---|---|---|
| | constant ∈ᵣ | |
| Solvent | (20° C.) | logK_{OW} |
| n-alkanes | approx. 1.8-2.0 | approx. 3-8 |
| iso-alkanes | approx. 1.8-1.9 | approx. 3-8 |
| Cycloalkanes | approx. 1.9-2.2 | approx. 3-8 |
| Benzene | approx. 2.3 | approx. 2.1 |
| Aromatics with | approx. 2.2-2.5 | approx. 2-6 |
| aliphatic radicals | | |
| Carbon tetrachloride | approx. 2.3 | approx. 2.8 |
| Halogenaromatics | approx. 5-10 | approx. 2-4 |
| Aromatics with | approx. 4-5 | approx. 2-3 |
| other residues | | |

An overview of some physical parameters of some specific examples of nonpolar organic solvents, in addition to those already shown in Table 2, is shown in Table 4 below (the values given are rounded values).

TABLE 4
Overview of some physical parameters for some
examples of nonpolar organic solvents.

| | Dielectric | Boiling | Melting | Steam | |
|---|---|---|---|---|---|
| | constant ∈ᵣ | point | point | pressure | |
| Solvent | (20° C.) | (° C.) | (° C.) | (20° C., hPa) | logK_{OW} |
| Benzene | 2.3 | 80 | 6 | 100 | 2.1 |
| Toluene | 2.4 | 111 | −95 | 29.1 | 2.7 |
| o-Xylene | 2.6 | 144 | −25 | <20 | 3.1 |
| m-Xylene | 2.4 | 139 | −48 | <20 | 3.2 |
| p-Xylene | 2.3 | 138 | 13 | 15 | 3.2 |
| Mesitylene | 2.4 | 165 | −45 | 2.69 | 3.4 |
| 1-Phenylbutane | 2.4 | 183 | −88 | 1.3 | 3.4 |
| 2-Methyl-1- | 2.4 | 173 | −51 | 1.8 | 3.4 |
| phenylpropane | | | | | |
| 2-Phenylbutane | 2.4 | 173 | −75 | 1.3 | 3.4 |
| 2-Methyl-2- | 2.4 | 169 | −58 | 2.2 | 3.4 |
| phenylpropane | | | | | |
| Cumol | 2.4 | 152 | −96 | 5.3 | 3.4 |
| Ethylbenzene | 2.4 | 136 | −95 | 9.8 | 3.2 |
| iso-butylbenzene | 2.2 | 77 | −23 | 1.8 | 4.1 |
| Carbon tetrachloride | 2.0 | 81 | 7 | 104 | 2.8 |
| Tetradecafluorohexane | 1.6 | 56 | −90 | 300 | 5.8 |
| Chlorobenzene | 5.6 | 132 | −46 | 18 | 2.9 |
| o-Dichlorobenzene | 9.9 | 180 | −17 | 1.3 | 3.4 |
| Fluorobenzene | 6.4 | 85 | −42 | 22.3 | 2.2 |
| Hexafluorobenzene | 2.1 | 81 | 4 | 77 | 2.6 |
| Anisol | 4.3 | 154 | −37 | 3.6 | 2.1 |
| Ethoxybenzene | 4.2 | 170 | −30 | 47 | 2.5 |

Preferred nonpolar organic solvents having a melting point of <20° C., a dielectric constant εᵣ at 20° C. of ≤3, a log K_{OW} >2.0, a boiling point of <200° C., a boiling point of >30° C., a vapor pressure at 20° C. between 1 hPa and 600 hPa include but are not limited to pentane, hexane, heptane, octane, nonane, decane, petroleum ether, isooctane, 2-methylpentane, 3-methylpentane, 2,2-dimethylbutane, 2,3-dimethylbutane, 2,2-dimethylpentane, 2-methylhexane, 3-methylhexane, 2,3-dimethylpentane, 2,4-dimethylpentane, 3,3-dimethylpentane, 3-ethylpentane, 2,2,4-trimethylpentane, 2,2,4-trimethylbutane, 2-methyloctane, 2-methylheptane, 3-methylheptane, 4-methylheptane, tetraethylmethane, cyclopentane, cyclohexane, methylcyclopentane, tert-butylcyclohexane, methylcyclohexane, 2,3-dimethylcyclobutane, cycloheptane, cyclooctane, cyclononane, cyclodecane, 1,2-dimethylcyclobutane, decalin, pinane, carbon tetrachloride, tetradecafluorohexane, hexafluorobenzene, benzene, toluene, o-xylene, m-xylene, p-xylene, mesitylene, ethylbenzene, 1 phenylbutane, 2-methyl-1-phenylpropane, 2-phenbutane, cumene, iso-butylbenzene, propylbenzene. Of course, mixtures of non-polar organic solvents can also be used.

Still further preferred nonpolar organic solvents having a melting point of <10° C., a dielectric constant εᵣ at 20° C. of <3, a log K_{OW} >2.0, a boiling point of <150° C., a boiling point of >30° C., a vapor pressure at 20° C. between 10 hPa and 600 hPa include but are not limited to pentane, hexane, heptane, octane, petroleum ether, isooctane, 2-methylpentane, 3-methylpentane, 2,2-dimethylbutane, 2,3-dimethylbutane, 2,2-dimethylpentane, 2-methylhexane, 3-methylhexane, 2,3-dimethylpentane, 2,4-dimethylpentane, 3,3-dimethylpentane, 3-ethylpentane, 2,2,4-trimethylpentane, 2,2,4-trimethylbutane, 2-methyloctane, 2-methylheptane, 3-methylheptane, 4-methylheptane, tetraethylmethane, cyclopentane, cyclohexane, methylcyclopentane, methylcyclohexane, cycloheptane, 2,3-dimethylcyclobutane, 1,2-dimethylcyclobutane, carbon tetrachloride, tetradecafluorohexane, hexafluorobenzene, benzene, toluene, o-xylene, m-xylene, p-xylene, ethylbenzene. Of course, mixtures of non-polar organic solvents can also be used.

Still further preferred nonpolar organic solvents having a melting point of <10° C., a dielectric constant εᵣ at 20° C. of ≤2.5, a log K_{OW} >2.0, a boiling point of <100° C., a boiling point of >40° C., a vapor pressure at 20° C. between 10 hPa and 300 hPa include but are not limited to hexane, heptane, 2-methylpentane, 3-methylpentane, 2,2-dimethylpentane, 2-methylhexane, 3-methylhexane, 2,3-dimethylpentane, 2,4-dimethylpentane, 3,3-dimethylpentane, 3-ethylpentane, 2,2,4-trimethylpentane, 2,2,4-trimethylbutane, cyclohexane, cycloheptane, 2,3-dimethylcyclobutane, 1,2-dimethylcyclobutane, carbon tetrachloride, tetradecafluorohexane, hexafluorobenzene, benzene. Of course, mixtures of non-polar organic solvents can also be used.

Still further preferred nonpolar organic solvents having a melting point of <10° C., a dielectric constant εᵣ at 20° C. of ≤2.5, a log K_{OW} >3.0, a boiling point of <100° C., a boiling point of >40° C., a vapor pressure at 20° C. between 10 hPa and 300 hPa include but are not limited to hexane, heptane, 2-methylpentane, 3-methylpentane, 2,2-dimethylpentane, 2-methylhexane, 3-methylhexane, 2,3-dimethylpentane, 2,4-dimethylpentane, 3,3-dimethylpentane, 3-ethylpentane, 2,2,4-trimethylpentane, 2,2,4-trimethylbutane, cyclohexane, cycloheptane, 2,3-dimethylcyclobutane, 1,2-dimethylcyclobutane, carbon tetrachloride. Of course, mixtures of non-polar organic solvents can also be used.

Preferred nonpolar organic solvents are also anhydrous, i.e., dried nonpolar organic solvents.

Preferred nonpolar organic solvents also have a density at normal temperature (20° C.) of <0.95 g/mL, more preferably <0.9 g/mL, even more preferably <0.8 g/mL. Thus, particularly preferred nonpolar organic solvents represent the non-solvents defined herein. Non-polar organic solvents particularly preferred herein are therefore hexane, cyclohexane and heptane.

Oils such as coconut oil, palm oil, peanut oil, cottonseed oil, canola oil, fish oil, soybean oil, flaxseed oil, olive oil are generally nonpolar and have a dielectric constant εᵣ at 20° C. of about 2-5. Oils such as castor oil, coconut oil, palm oil, peanut oil, cottonseed oil, canola oil, fish oil, soybean oil, flaxseed oil, olive oil are less preferred herein as nonpolar organic solvents. These oils are viscous and have a viscosity at 20° C. of about 30-160 mPa s. Non-polar organic solvents preferred herein have a viscosity at normal temperature (20° C.) of <2.0 mPa s, more preferably <1.5 mPa s, even more preferably <1.0 mPa s.

The term “polar organic solvent”, as used herein, refers to a carbon-based solvent that is liquid at normal temperature (20° C.) and pressure (101 hPa; 1 bar, 1 atm), i.e., has at least a melting point of <20° C. Examples of common polar organic solvents include, but are not limited to, acetonitrile, dimethyl sulfoxide, ethers such as dioxane, tetrahydrofuran (THF), diethyl ether, methyl tert-butyl ether (MTDC), ketones such as acetone, butanone or pentanone, alcohols such as methanol, ethanol, propanol, isopropanol, carboxylic acids such as formic acid, acetic acid, propionic acid, amides such as dimethylformamide (DMF) or dimethylacetamide, halogenated solvents such as chloroform, methylene chloride, and carboxylic acid esters such as methyl acetate, ethyl acetate.

Polar organic solvents, as defined herein, preferably exhibit an n-octanol-water partition coefficient log K_{OW} of <+2.0, preferably from −1.0 to +2.0, and more preferably from −0.5 to +1.5, further preferably from −0.4 to +1.4, even more preferably from −0.4 to +0.9. Further preferred polar organic solvents exhibit a dielectric constant εᵣ at 20° C. of >3, more preferably of 5.0. Preferred polar organic solvents also exhibit a dielectric constant εᵣ at 20° C. of <50, more preferably of <40, still more preferably of <35, most preferably of <30.

Organic solvents preferred herein thus have an n-octanol-water partition coefficient log K_{OW} of ≤+2.0, and a dielectric constant εᵣ at 20° C. of >3, still more preferably a log K_{OW} of −1.0 to +2.0 and a dielectric constant εᵣ at 20° C. of ≥5.0 and <40, more preferably a log K_{OW} of −0.5 to +1.5 and a dielectric constant εᵣ at 20° C. of ≥5.0 and <30, and most preferably a log K_{OW} of −0.4 to +1.4 and a dielectric constant εᵣ at 20° C. of ≥5.0 and <30. Preferred organic solvents thus exhibit an n-octanol-water partition coefficient log K_{OW} of −1.0 to +2.0 and a dielectric constant εᵣ at 20° C. of >3.0 to 40, even more preferably a log K_{OW} of −1.0 to +2.0 and a dielectric constant εᵣ at 20° C. of 5.0 to 40, even more preferably a log K_{OW} of −1.0 to +2.0 and a dielectric constant εᵣ at 20° C. from 5.0 to 35, and most preferably a log K_{OW} from −0.5 to +1.5 and a dielectric constant εᵣ at 20° C. from 5.0 to 30. Thus, a solvent with a dielectric constant εᵣ at 20° C. of >3 and a log K_{OW} >2.0 is not a polar organic solvent herein. For example, chlorobenzene has a dielectric constant εᵣ at 20° C. of about 5.6 but a log K_{OW} of about 2.9 and thus does not constitute a polar organic solvent herein.

Polar organic solvents suitable for the present invention exist in the liquid state of aggregate at normal temperature (20° C.) and normal pressure (101 hPa; 1 bar, 1 atm). Preferred polar organic solvents have a melting point of <20° C., more preferably <15° C., still more preferably <10° C. Preferred polar organic solvents also exhibit a boiling point of <200° C., further preferably <150° C., even more preferably <100° C. Preferred polar organic solvents also exhibit a boiling point of >25° C., more preferably >30° C., even more preferably of >40° C. Preferred polar organic solvents thus exhibit a boiling point of between 25° C. and 200° C., further preferably between 30° C. and 150° C., still more preferably between 40° C. and 100° C. The indications on melting points and boiling points refer here to normal pressure (101 hPa; 1 bar, 1 atm).

Preferred polar organic solvents also exhibit a vapor pressure at normal temperature (20° C.) of <600 hPa, more preferably <300 hPa, even more preferably <200 hPa. Preferred polar organic solvents further exhibit a vapor pressure at 20° C. of >1 hPa, more preferably >10 hPa, even more preferably >30 hPa. Preferred polar organic solvents thus exhibit a vapor pressure at normal temperature (20° C.) of between 1 hPa to 600 hPa, more preferably between 10 hPa and 300 hPa, still more preferably between 30 hPa and 200 hPa.

Preferred polar organic solvents exhibit a dipole moment of ≥1.0 D (3.3·10⁻³⁰ Cm), further preferred polar organic solvents exhibit a dipole moment of <3.0 D (9.9·10⁻³⁰ Cm), even more preferred ≤2.0 D (6.6·10⁻³⁰ Cm).

The term “polar organic solvent”, as used herein, refers to aprotic polar solvents and protic solvents. In aprotic polar solvents, the molecule is asymmetrically substituted so that the molecule has a dipole moment. Examples of aprotic polar solvents include ethers, esters, acid anhydrides, ketones, e.g. acetone, tertiary amines, pyridine, furan, thiophene, asymmetric halogenated hydrocarbons, nitromethane, dimethylformamide (DMF), dimethyl sulfoxide (DMSO), dimethyl carbonate, tetramethyl urea, tetraethyl urea, dimethyl propylene urea (DMPU), 1,3-dimethyl-2-imidazolidinone (DMEU). The most important protic solvent is water. Examples of other protic solvents are alcohols, aldehydes and carboxylic acids. Thus, protic solvents are water, methanol, ethanol and other alcohols, primary and secondary amines, carboxylic acids such as formic acid and acetic acid, formamide.

Preferred polar organic solvents include, in particular, the aprotic polar solvents, as these are generally miscible with the non-polar organic solvents as defined herein, in particular the non-solvents as defined herein, in any mixing ratio.

An overview of orientation values (the values given are rounded values) of the dielectric constants and log K_{OW} for some polar organic solvents is shown in Table 5.

TABLE 5
Overview of dielectric constants and logK_{OW} for
some polar organic solvents (rounded values)

| | Dielectric | |
|---|---|---|
| Solvent | constant ∈ᵣ | |
| (rounded values) | (20° C.) | logK_{OW} |
| Halogenalkanes | approx. 3-11 | approx. +1.0-+1.98 |
| Alkylether | approx. 4-8 | approx. −0.4-+1.98 |
| Alkyl ketones | approx. 15-25 | approx. −0.5-+1.5 |
| Pyridine | approx. 9-13 | approx. +0.6 |
| Alkylnitrile | approx. 25-38 | approx. −0.3-+0.5 |
| Alkylamines | approx. 3-7 | approx. −0.8 |
| Alkyldiols | approx. 30-50 | approx. −0.9 |
| Higher alkyl alcohols | approx. 5-17 | approx. +0.8-≥+2.0 |
| Lower alkyl alcohols | ca. 19-32 | approx. −0.7-+0.3 |
| Aldehydes | approx. 9-18 | approx. −4.0 |
| Formic acid | approx. 50 | approx. −0.5 |
| Higher carboxylic acids | approx. 2-6 | approx. −0.2 |
| Alkyl ester | approx. 3-9 | approx. +0.0-≥+2.0 |
| Water | approx. 80 | approx. −1.4 |
| DMF | approx. 37 | approx. −1.0 |
| DMSO | approx. 47 | approx. −1.4 |

An overview of some physical parameters (the values given are rounded values) of some polar organic solvents is shown in Table 6 below.

TABLE 6
Overview of some physical parameters for
some examples of polar organic solvents.

| | Dielectric | Boiling | Melting | Steam | |
|---|---|---|---|---|---|
| | constant ∈ᵣ | point | point | pressure | |
| Solvent | (20° C.) | (° C.) | (° C.) | (20° C., hPa) | logK_{OW} |
| Dichloromethane | 9.0 | 40 | −95 | 475 | +1.3 |
| Chloroform | 4.8 | 61 | −64 | 210 | +2.0 |
| Diethyl ether | 4.3 | 35 | −117 | 586 | +0.9 |
| Dipropyl ether | 3.3 | 90 | −122 | 73 | +2.0 |
| 2-Pentanone | 15.4 | 101 | −78 | | +0.9 |
| Tetrahydrofuran (THF) | 7.6 | 66 | −−109 | 173 | +0.5 |
| 1,4-Dioxane | 2.3 | 101 | −12 | 38 | −0.4 |
| Acetone | 20.7 | 56 | −95 | 246 | −0.2 |
| Acetonitrile | 37 | 82 | −44 | 94 | −0.3 |
| Nitromethane | 37 | 101 | −29 | 36 | −0.4 |
| Acetic acid | 6.2 | 118 | −17 | 15.8 | −0.2 |
| Methanol | 32.8 | 65 | −98 | 129 | −0.7 |
| Ethanol | 24.9 | 78 | −114 | 58 | −0.3 |
| 1-Propanol | 20.2 | 97 | −126 | 104 | +0.3 |
| iso-propanol | 19.9 | 82 | −88 | 44 | +0.1 |
| Ethylene glycol | 37.0 | 197 | −16 | 22.3 | −1.4 |
| Glycerin | 47.0 | 290 | 18 | 77 | −1.8 |
| Methyl acetate | 7.1 | 57 | −99 | 228 | +0.2 |
| Ethyl acetate | 6.0 | 77 | −84 | 98 | +0.7 |
| n-Propyl acetate | 5.6 | 102 | −95 | 33 | +1.4 |
| DMF | 37 | 153 | −60 | 3.8 | −1.0 |
| Dimethylacetamide | 38 | 166 | −20 | 3.3 | −0.8 |
| N-methylpyrrolidone | 32 | 203 | −24 | 0.3 | −0.5 |
| DMSO | 47 | 189 | 18 | 0.6 | −1.4 |
| Water | 80 | 100 | 0 | 23 | |

Preferred polar organic solvents are tetrahydrofuran, acetone, methanol, ethanol, n-propanol, iso-propanol chloroform, methylene chloride (dichloromethane) and ethyl acetate (ethyl acetate).

Further preferred polar organic solvents are tetrahydrofuran, acetone, ethanol, n-propanol, iso-propanol and ethyl acetate.

Most preferred are the physiologically largely harmless solvents ethanol, iso-propanol and ethyl acetate. Ethyl acetate is particularly preferred.

Of course, mixtures of polar organic solvents can also be used.

Water is considered a very polar organic solvent, but should be avoided because water-containing coatings are difficult to dry. In addition, the tri-O-acylglycerols according to the invention are poorly soluble or virtually insoluble in very polar solvents such as water. A suspension containing only water as solvent is not according to the invention, since the tri-O-acylglycerols selected from the group consisting of trioctanoylglycerol, trinonanoylglycerol, tridecanoylglycerol, and triundecanoylglycerol cannot exist in dissolved form in water. However, water-containing solvent mixtures of water-miscible polar organic solvents in which the at least one tri-O-acylglycerol selected from the group consisting of trioctanoylglycerol, trinonanoylglycerol, tridecanoylglycerol, and triundecanoylglycerol are present in dissolved form can also be provided, e. g. solvent mixtures such as water/methanol (25:75), water/isopropanol (35:65) or also water/acetonitrile (15:85). Nevertheless, anhydrous solvent mixtures are preferred herein.

Particularly preferred are therefore anhydrous suspensions for coating a medical device, preferably selected from a catheter balloon, a balloon catheter, a stent, or a cannula, containing at least one tri-O-acylglycerol selected from the group consisting of trioctanoylglycerol, trinonanoylglycerol, tridecanoylglycerol, and triundecanoylglycerol and at least one limus active agent in the form of microcrystals; and a solvent or a solvent mixture, wherein the at least one tri-O-acylglycerol is dissolved in the at least one solvent or the solvent mixture, and wherein the microcrystals of the at least one limus active agent do not dissolve in the solvent or the solvent mixture containing the dissolved at least one tri-O-acylglycerol.

An “anhydrous suspension” as used herein contains no more than 20% by volume of water, preferably less than 20% by volume, more preferably less than 10% by volume, more preferably less than 5% by volume, more preferably less than 3% by volume, more preferably less than 2% by volume, more preferably less than 1.5% by volume, still more preferably less than 1% by volume, still more preferably less than 0.5% by volume, and most preferably less than 0.1% by volume of water based on the total volume of the suspension.

In some preferred embodiments, the suspension of the present invention comprises a solvent mixture wherein the at least one tri-O-acylglycerol dissolves and the microcrystals of the at least one limus active agent do not dissolve. Preferred solvent mixtures herein are mixtures of at least one non-polar organic solvent, preferably at least one non-solvent and at least one polar organic solvent. Non-polar organic solvent or non-solvent and polar organic solvent must be miscible with each other and preferably miscible with each other in any ratio to give a homogeneous mixture.

The non-polar organic solvents, as defined herein, are generally immiscible with water and other highly polar organic solvents such as short chain alcohols such as methanol in any ratio. Non-polar organic solvents that are immiscible with water include, but are not limited to, benzene, carbon tetrachloride, cyclohexane, heptane, hexane, isooctane, pentane, toluene, and xylene. Thus, in particular, the non-solvents as defined herein are immiscible with water. Solvent mixtures containing at least one non-solvent therefore particularly preferably do not contain water as a mixture component. Furthermore, some non-polar organic solvents such as xylene, cyclohexane, heptane, hexane, isooctane and pentane are not miscible in any ratio even with very polar organic solvents such as dimethyl sulfoxide and dimethyl formamide. In addition, the non-solvents as defined herein, such as cyclohexane, heptane, hexane, isooctane and pentane are also immiscible with polar organic solvents such as acetonitrile and methanol.

Solvent mixtures of at least one non-solvent, as defined herein, and at least one polar organic solvent therefore particularly preferably contain polar organic solvents having a log K_{OW} of −0.5 to +1.5 and a dielectric constant εᵣ at 20° C. of <30, further preferably a log K_{OW} of −0.4 to +1.4 and a dielectric constant εᵣ at 20° C. of <30.

In some embodiments, the volume ratio in the suspension between the nonpolar organic solvent and the polar organic solvent is between 25:75 to 75:25, preferably between 30:70 to 70:30, and more preferably between 35:65 and 65:35.

Preferably, the volume ratio in the suspension between the nonpolar organic solvent and the polar organic solvent is between 99:1 to 65:35, preferably between 95:5 to 70:30, and more preferably between 80:20 and 65:35, more preferably between 90:10 and 80:15 and most preferably 85:15.

Further preferred are solvent mixtures containing at least 50% by volume nonpolar organic solvent, more preferably at least 55% by volume nonpolar organic solvent, more preferably at least 60% by volume nonpolar organic solvent, more preferably at least 65% by volume nonpolar organic solvent, more preferably at least 70% by volume nonpolar organic solvent, more preferably at least 75% by volume nonpolar organic solvent, and most preferably at least 80% by volume nonpolar organic solvent.

Further preferred are solvent mixtures containing at least 50% by volume non-solvent, more preferably at least 55% by volume non-solvent, more preferably at least 60% by volume non-solvent, more preferably at least 65% by volume non-solvent, more preferably at least 70% by volume non-solvent, more preferably at least 75% by volume non-solvent and most preferably at least 80% by volume non-solvent.

In preferred embodiments, the solvent mixture therefore contains at least one non-polar organic solvent having a dielectric constant εᵣ at 20° C. of ≤10 and an n-octanol-water partition coefficient log K_{OW} of >2.0, more preferably having a dielectric constant εᵣ at 20° C. of ≤5.0 and an n-octanol-water partition coefficient log K_{OW} of ≥2.5, more preferably having a dielectric constant εᵣ at 20° C. of ≤3.0 and an n-octanol-water partition coefficient log K_{OW} of ≥3.0, and most preferably having a dielectric constant εᵣ at 20° C. of ≤2.0.

In further preferred embodiments, the solvent mixture contains at least 50% by volume, more preferably at least 55% by volume, more preferably at least 60% by volume, more preferably at least 65% by volume, more preferably at least 70% by volume, more preferably at least 75% by volume and most preferably at least 80% by volume of % non-polar organic solvent having a dielectric constant εᵣ at 20° C. of ≤10 and an n-octanol-water partition coefficient log K_{OW} of >2.0, more preferably having a dielectric constant εᵣ at 20° C. of <5.0, and an n-octanol-water partition coefficient log K_{OW} of ≥2.5, more preferably having a dielectric constant εᵣ at 20° C. of ≤3.0 and an n-octanol water partition coefficient log K_{OW} of ≥3.0 and most preferably having a dielectric constant εᵣ at 20° C. of ≤2.0.

A preferred combination of polar organic solvent and non-polar organic solvent is, for example, ethanol and cyclohexane or ethyl acetate and heptane.

Preferred are solvent mixtures of at least one polar organic solvent having a log K_{OW} of −1.0 to +2.0 and a dielectric constant εᵣ at 20° C. of 3.0 to 40, still more preferably a log K_{OW} of −1.0 to +2.0 and a dielectric constant εᵣ at 20° C. of 5.0 to 40, still more preferably a log K_{OW} of −1.0 to +2.0 and a dielectric constant εᵣ at 20° C. of 5.0 to 35, more preferably a log K_{OW} of −0.5 to +1.5 and a dielectric constant εᵣ at 20° C. of 5.0 to 30, and most preferably a log K_{OW} of −0.4 to +0.9 and a dielectric constant εᵣ at 20° C. of 5.0 to 30, and a nonpolar organic solvent having a dielectric constant εᵣ at 20° C. of ≤10 and an n-octanol-water partition coefficient log K_{OW} of >2.0, more preferably having a dielectric constant εᵣ at 20° C. of ≤5.0 and an n-octanol-water partition coefficient log K_{OW} of ≥2.5, more preferably having a dielectric constant εᵣ at 20° C. of ≤3.0 and an n-octanol-water partition coefficient log K_{OW} of ≥3.0, and most preferably having a dielectric constant εᵣ at 20° C. of ≤2.0.

Preferred are solvent mixtures of at least one polar organic solvent selected from the group comprising or consisting of tetrahydrofuran, acetone, methanol, ethanol, n-propanol, iso-propanol, chloroform, dichloromethane, ethyl acetate, preferably tetrahydrofuran, acetone, ethanol, n-propanol, iso-propanol, and ethyl acetate, further preferably ethanol, iso-propanol, and ethyl acetate, and a nonpolar organic solvent having a dielectric constant εᵣ at 20° C. of ≤10 and an n-octanol-water partition coefficient log K_{OW} of >2.0, more preferably having a dielectric constant εᵣ at 20° C. of ≤5.0, and an n-octanol-water partition coefficient log K_{OW} of ≥2.5, more preferably having a dielectric constant εᵣ at 20° C. of from ≤3.0 and an n-octanol-water partition coefficient log K_{OW} of ≥3.0, and most preferably having a dielectric constant εᵣ at 20° C. of ≤2.0.

A particularly preferred combination of polar organic solvent and nonpolar organic solvent is a solvent mixture of ethyl acetate and a nonpolar organic solvent as defined herein having a dielectric constant εᵣ at 20° C. of ≤10 and an n-octanol-water partition coefficient log K_{OW} of >2.0, more preferably having a dielectric constant εᵣ at 20° C. of <5.0 and an n-octanol-water partition coefficient log K_{OW} of ≥2.5, more preferably having a dielectric constant εᵣ at 20° C. of ≤3.0 and an n-octanol-water partition coefficient log K_{OW} of ≥3.0, and most preferably having a dielectric constant εᵣ at 20° C. of ≤2.0.

An even more preferred combination of polar organic solvent and non-polar organic solvent is a solvent mixture of ethyl acetate and a non-solvent as defined herein having a dielectric constant εᵣ at 20° C. of ≤2.0.

Preferred combinations of polar organic solvent and nonpolar organic solvent are ethyl acetate and a nonpolar organic solvent selected from the group comprising or consisting of pentane, hexane, heptane, octane, nonane, decane, petroleum ether, isooctane, 2-methylpentane, 3-methylpentane, 2,2-dimethylbutane, 2,3-dimethylbutane, 2,2-dimethylpentane, 2-methylhexane, 3-methylhexane, 2,3-dimethylpentane, 2,4-dimethylpentane, 3,3-dimethylpentane, 3 ethylpentane, 2,2, 4-trimethylpentane, 2,2,4-trimethylbutane, 2-methyloctane, 2-methylheptane, 3-methylheptane, 4-methylheptane, tetraethylmethane, cyclopentane, cyclohexane, methylcyclopentane, tert-butylcyclohexane, methylcyclohexane, 2,3 dimethylcyclobutane, cycloheptane, cyclooctane, cyclononane, cyclodecane, 1,2-dimethylcyclobutane, decalin, pinane, carbon tetrachloride, tetradecafluorohexane, hexafluorobenzene, benzene, toluene, o-xylene, m-xylene, p-xylene, mesitylene, ethylbenzene, 1-phenylbutane, 2-methyl-1-phenylpropane, 2-phenbutane, cumene, iso-butylbenzene, propylbenzene, preferably pentane, hexane, heptane, octane, petroleum ether, isooctane, 2-methylpentane, 3-methylpentane, 2,2-dimethylbutane, 2,3-dimethylbutane, 2,2-dimethylpentane, 2-methylhexane, 3-methylhexane, 2,3-dimethylpentane, 2,4-dimethylpentane, 3,3-dimethylpentane, 3-ethylpentane, 2,2,4-trimethylpentane, 2,2,4-trimethylbutane, 2-methyloctane, 2-methylheptane, 3-methylheptane, 4-methylheptane, tetraethylmethane, cyclopentane, cyclohexane, methylcyclopentane, methylcyclohexane, cycloheptane, 2,3-dimethylcyclobutane, 1,2-dimethylcyclobutane, carbon tetrachloride, tetradecafluorohexane, hexafluorobenzene, benzene, toluene, o-xylene, m-xylene, p-xylene, ethylbenzene, further preferably hexane, heptane, 2-methylpentane, 3-methylpentane, 2,2-dimethylpentane, 2-methylhexane, 3-methylhexane, 2,3-dimethylpentane, 2,4-dimethylpentane, 3,3-dimethylpentane, 3-ethylpentane, 2,2,4-trimethylpentane, 2,2,4-trimethylbutane, cyclohexane, cycloheptane, 2,3-dimethylcyclobutane, 1,2-dimethylcyclobutane, carbon tetrachloride, tetradecafluorohexane, hexafluorobenzene, benzene, still further preferably hexane, heptane, 2-methylpentane, 3-methylpentane, 2,2-dimethylpentane, 2-methylhexane, 3-methylhexane, 2,3-dimethylpentane, 2,4-dimethylpentane, 3,3-dimethylpentane, 3-ethylpentane, 2,2,4-trimethylpentane, 2,2,4-trimethylbutane, cyclohexane, cycloheptane, 2,3-dimethylcyclobutane, 1,2-dimethylcyclobutane, carbon tetrachloride, and most preferably hexane, heptane and cyclohexane.

K_{OW} is the n-octanol-water partition coefficient. The K_{OW} is thus the partition coefficient of a substance in the two-phase system of n-octanol and water. The log K_{OW} is the decadic logarithm of the K_{OW}. The log K_{OW} is also known as log P in English-speaking countries. The K_{OW} serves as a measure of the relationship between lipophilicity and hydrophilicity of a substance. The value is greater than one if a substance is more soluble in lipophilic solvents such as n-octanol, less than one if it is more soluble in water.

n-Octanol-water partition coefficients log K_{OW} of various solvents are well known to those skilled in the art, see for example James Sangster, “Octanol-Watter Partition Coefficients of Simple Organic Compounds,” J. Phys. Chem. Ref. Data 1989, Vol. 18, No. 3, pp. 1111-1227, which is incorporated herein by reference in its entirety. Polar organic solvents are defined herein as those having a log K_{OW} of −1.0 to +2.0 and preferably of −0.5 to +1.5. Non-solvents or non-polar organic solvents are referred to herein as those having a log K_{OW} ≥2.8 preferably a log K_{OW} ≥3.3 or a log K_{OW} from +2.8 to +7.5 and preferably from +3.3 to +7.0.

Measurement methods for determining n-octanol-water partition coefficients are also well known to those skilled in the art, see for example James Sangster, “Octanol-Watter Partition Coefficients of Simple Organic Compounds”, J. Phys. Chem. Ref. Data 1989, Vol. 18, No. 3, pp. 1111-1227, in the section “Methods of Measurement”. A practical determination of the log K_{OW} value can be carried out in such a way that the respective solvent with a known concentration c_{0b}^{water} is introduced into aqueous solution at a known volume overlaid with a precisely measured volume of octanol V^{Octanyl} and intensively mixed. Phase separation is then waited for and the octanol phase is separated. To ensure that no further volume change occurs during phase mixing, the octanol used is saturated in advance with water and the water used is saturated in advance with octanol. The log K_{OW} is positive for lipophilic and negative for hydrophilic solvents.

TABLE 7
Overview logK_{OW} values of some polar organic solvents

| Solvent | logK_{OW} | Solvent | logK_{OW} |
|---|---|---|---|
| Acetonitrile | −0.34 | Acetone | −0.24 |
| Dimethyl sulfoxide | −1.35 | 2-Butanone | 0.29 |
| Dioxane | −0.42 | 2-Pentanone | 0.84 |
| Tetrahydrofuran | 0.46 | Methanol | −0.74 |
| (THF) | | | |
| Ethyl acetate | 0.73 | Ethanol | −0.30 |
| Diethyl ether | 0.89 | 1-Propanol | 0.25 |
| Methyl tert-butyl | 0.94 | 2-Propanol | 0.05 |
| ether (MTDC) | | | |
| Dimethylformamide | −1.01 | 1-Butanol | 0.84 |
| (DMF) | | | |
| Dimethylacetamide | −0.77 | tert-butyl alcohol | 0.35 |
| Methylene chloride | 1.25 | Acetic acid | −0.17 |
| Chloroform | 1.97 | Propionic acid | 0.32 |
| Formic acid | −0.54 | | |

TABLE 8
Overview of logK_{OW}-values of some nonpolar organic solvents

| Solvent | logK_{OW} | Solvent | logK_{OW} |
|---|---|---|---|
| Toluene | 2.73 | Pentane | 3.45 |
| Benzene | 2.13 | Cyclopentane | 3.00 |
| Cyclohexane | 2.86 | Hexane | 4.00 |
| Nonan | 5.65 | Heptane | 4.50 |
| Decan | 6.25 | Octane | 5.15 |
| o-Xylene | 3.12 | Mesitylene | 3.42 |
| m-Xylene | 3.20 | Petroleum ether | 4.20 |
| p-Xylene | 3.15 | | |

Between the log K_{OW} value of the polar organic solvent and the log K_{OW} value of the nonpolar organic solvent there should be at least a difference of 1.0, preferably at least 1.5, and more preferably at least 2.0.

To determine the K_{OW} of a mixture of nonpolar organic solvents, the K_{OW} values of the individual nonpolar organic solvents are weighted according to the volume fraction of the mixture and the mean value is determined from the weighted K_{OW} values.

The dielectric constant (relative permittivity, formula symbol εᵣ) is a physical substance constant that can be used to describe certain properties of solvents. Solvents with high dielectric constant are good solvents for ionic and other polar compounds, those with low dielectric constant are better solvents for non-polar compounds. The term “dielectric constant” is also referred to in the prior art as permittivity, dielectric conductivity, dielectricity, or dielectric function. The relative permittivity εᵣ of a medium, also called permittivity or dielectric constant, is the dimensionless ratio of the permittivity E to the permittivity ε₀ of the vacuum. For gaseous, liquid and solid matter, εᵣ >1. Measurement methods for determining the relative permittivity are well known to those skilled in the art. There are many methods that have been developed for measuring the dielectric constant. For example, the open coaxial probe method is suitable for liquids. In this method, the probe is immersed in the liquids and the reflection coefficient is measured and used to determine the dielectric constant.

According to the present invention, the suspension according to the invention contains a solvent or a solvent mixture. According to the invention, at least one tri-O-acylglycerol selected from the group consisting of trioctanoylglycerol, trinonanoylglycerol, tridecanoylglycerol, and triundecanoylglycerol is dissolved in the solvent or in the solvent mixture. According to the invention, the suspension therefore contains a solvent or a solvent mixture in which at least one tri-O-acylglycerol selected from the group consisting of trioctanoylglycerol, trinonanoylglycerol, tridecanoylglycerol, and triundecanoylglycerol is dissolved, wherein the microcrystals of the at least one limus active agent do not dissolve or no longer dissolve in the presence of the at least one tri-O-acylglycerol.

Thus, by definition, the solvent or solvent mixture forms a solution with the dissolved at least one tri-O-acylglycerol selected from trioctanoylglycerol, trinonanoylglycerol, tridecanoylglycerol, and triundecanoylglycerol and thus constitutes a homogeneous mixture. The solution of at least one tri-O-acylglycerol selected from trioctanoylglycerol, trinonanoylglycerol, tridecanoylglycerol, and triundecanoylglycerol in the solvent or solvent mixture thus has only one phase and the dissolved at least one tri-O-acylglycerol selected from trioctanoylglycerol, trinonanoylglycerol, tridecanoylglycerol, and triundecanoylglycerol is uniformly distributed in the solvent or solvent mixture.

Thus, the suspension according to the present invention is a combination of:
1) a solution of at least one tri-O-acylglycerol selected from the group consisting of trioctanoylglycerol, trinonanoylglycerol, tridecanoylglycerol, and triundecanoylglycerol in a solvent or a solvent mixture, and
2) at least one limus active agent in the form of microcrystals, wherein the microcrystals of the at least one limus active agent do not dissolve in the solution according to 1).

The at least one limus active agent in the form of microcrystals is finely distributed as a solid, i.e. “suspended”, in the solution of at least one tri-O-acylglycerol selected from the group consisting of trioctanoylglycerol, trinonanoylglycerol, tridecanoylglycerol, and triundecanoylglycerol in at least one solvent or a solvent mixture. Thus, according to the invention, the microcrystals of the at least one limus active agent are “suspended” in the solution of at least one tri-O-acylglycerol selected from the group consisting of trioctanoylglycerol, trinonanoylglycerol, tridecanoylglycerol, and triundecanoylglycerol in a solvent or a solvent mixture.

Thus, the present invention also relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol;
b) at least one limus active agent in the form of microcrystals; and
c) a solvent or a mixture of solvents,
   wherein the at least one tri-O-acylglycerol is dissolved in the solvent or solvent mixture, and
   wherein the microcrystals of the at least one limus active agent do not dissolve in the solvent or solvent mixture containing the dissolved at least one tri-O-acylglycerol.

With other words, the present invention relates to a suspension for coating of a medical device, preferably selected from a catheter balloon, a balloon catheter, a stent, or a cannula, the suspension containing:
a) a solution of at least one of trioctanoylglycerol, trinonanoylglycerol, tridecanoylglycerol, and triundecanoylglycerol; and
b) at least one limus active agent in the form of microcrystals, the microcrystals being suspended in said solution.

With still other words, the present invention relates to a suspension for coating of a medical device, preferably selected from a catheter balloon, a balloon catheter, a stent, or a cannula, the suspension containing:
a) a solution of at least one of trioctanoylglycerol, trinonanoylglycerol, tridecanoylglycerol, and triundecanoylglycerol, and
b) microcrystals of at least one limus active agent suspended in said solution.

The present invention therefore relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals and
c) a solvent or a solvent mixture, in which the at least one tri-O-acylglycerol dissolves and in which the microcrystals of the at least one limus active agent do not dissolve,
   wherein the microcrystals of the at least one limus active agent have a crystal size in the range of 1 μm to 300 μm.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
   wherein the at least one limus active agent has a crystallinity of at least 90% by weight.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
   wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
wherein the at least one limus active agent is selected from rapamycin (sirolimus), and everolimus.

The present invention relates to a suspension for coating a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
wherein the microcrystals of the at least one limus active agent have a crystal size in the range of 1 μm to 300 μm,
   wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
   wherein the at least one limus active agent has a crystallinity of at least 90% by weight,
   wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
   wherein the microcrystals of the at least one limus active agent have a crystal size in the range of 1 μm to 300 μm,
   wherein the at least one limus active agent is selected from rapamycin (sirolimus) and everolimus.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
   wherein the at least one limus active agent has a crystallinity of at least 90% by weight,
   wherein the at least one limus active agent is selected from rapamycin (sirolimus), and everolimus.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
   wherein the microcrystals of the at least one limus active agent have a crystal size in the range of 1 μm to 300 μm,
   wherein the at least one limus active agent has a crystallinity of at least 90% by weight,
   wherein the at least one limus active agent is selected from rapamycin (sirolimus), and everolimus.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
wherein the at least one tri-O-acylglycerol and the at least one limus active agent are present in a mass ratio of 10%-30% tri-O-acylglycerol to 90%-70% limus active agent.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
wherein the microcrystals of the at least one limus active agent have a crystal size in the range of 1 μm to 300 μm,
   wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus zotarolimus,
   wherein the at least one tri-O-acylglycerol and the at least one limus active agent are present in a mass ratio of 10%-30% tri-O-acylglycerol to 90%-70% limus active agent.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
   wherein the at least one limus active agent has a crystallinity of at least 90% by weight,
   wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus,
   wherein the at least one tri-O-acylglycerol and the at least one limus active agent are present in a mass ratio of 10%-30% tri-O-acylglycerol to 90%-70% limus active agent.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
wherein the microcrystals of the at least one limus active agent have a crystal size in the range of 1 μm to 300 μm,
   wherein the at least one limus active agent is selected from rapamycin (sirolimus), and everolimus,
   wherein the at least one tri-O-acylglycerol and the at least one limus active agent are present in a mass ratio of 10%-30% tri-O-acylglycerol to 90%-70% limus active agent.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
   wherein the at least one limus active agent has a crystallinity of at least 90% by weight,
   wherein the at least one limus active agent is selected from rapamycin (sirolimus) and everolimus,
   wherein the at least one tri-O-acylglycerol and the at least one limus active agent are present in a mass ratio of 10%-30% tri-O-acylglycerol to 90%-70% limus active agent.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
wherein the microcrystals of the at least one limus active agent have a crystal size in the range of 1 μm to 300 μm,
   wherein the solvent is a non-solvent having a dielectric constant εᵣ at 20° C. of <2.0 or the solvent mixture contains at least 50% by volume of a non-solvent having a dielectric constant εᵣ at 20° C. of <2.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent mixture, in which the at least one tri-O-acylglycerol dissolves and in which the microcrystals of the at least one limus active agent do not dissolve,
wherein the microcrystals of the at least one limus active agent have a crystal size in the range of 1 μm to 300 μm,
   wherein the solvent mixture is a mixture of at least one polar organic solvent having an n-octanol-water partition coefficient log K_{OW} of from −0.5 to +1.5 and a dielectric constant εᵣ at 20° C. of from 5.0 to 30 and at least one non-polar organic solvent having a dielectric constant εᵣ at 20° C. of from ≤3.0 and an n-octanol-water partition coefficient log K_{OW} of ≥3.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent mixture, in which the at least one tri-O-acylglycerol dissolves and in which the microcrystals of the at least one limus active agent do not dissolve,
wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus,
   wherein the solvent mixture is a mixture of at least one polar organic solvent having an n-octanol-water partition coefficient log K_{OW} of from −0.5 to +1.5 and a dielectric constant εᵣ at 20° C. of from 5.0 to 30 and at least one non-polar organic solvent having a dielectric constant εᵣ at 20° C. of from ≤3.0 and an n-octanol-water partition coefficient log K_{OW} of ≥3.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, temsirolimus and zotarolimus,
   wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
wherein the at least one limus active agent is selected from rapamycin (sirolimus), and everolimus,
   wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent mixture, in which the at least one tri-O-acylglycerol dissolves and in which the microcrystals of the at least one limus active agent do not dissolve,
wherein the at least one limus active agent is selected from rapamycin (sirolimus) and everolimus,
   wherein the solvent mixture is a mixture of at least one polar organic solvent having an n-octanol-water partition coefficient log K_{OW} of −0.5 to +1.5 and a dielectric constant εᵣ at 20° C. of 5.0 to 30 and at least one non-polar organic solvent having a dielectric constant εᵣ at 20° C. of ≤3.0 and an n-octanol-water partition coefficient log K_{OW} of ≥3.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
wherein the microcrystals of the at least one limus active agent have a crystal size in the range of 1 μm to 300 μm,
   wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus,
   wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

The present invention relates to a suspension for coating of a medical device, preferably selected from a catheter balloon, a balloon catheter, a stent, or a cannula, the suspension containing: a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent mixture, in which the at least one tri-O-acylglycerol dissolves and in which the microcrystals of the at least one limus active agent do not dissolve,
   wherein the microcrystals of the at least one limus active agent have a crystal size in the range of 1 μm to 300 μm,
   wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus,
   wherein the solvent mixture is a mixture of at least one polar organic solvent having an n-octanol-water partition coefficient log K_{OW} of from −0.5 to +1.5 and a dielectric constant εᵣ at 20° C. of from 5.0 to 30 and at least one non-polar organic solvent having a dielectric constant εᵣ at 20° C. of from ≤3.0 and an n-octanol-water partition coefficient log K_{OW} of ≥3.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
wherein the at least one limus active agent has a crystallinity of at least 90% by weight,
   wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus,
   wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent mixture, in which the at least one tri-O-acylglycerol dissolves and in which the microcrystals of the at least one limus active agent do not dissolve,
wherein the at least one limus active agent has a crystallinity of at least 90% by weight,
   wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus,
   wherein the solvent mixture is a mixture of at least one polar organic solvent having an n-octanol-water partition coefficient log K_{OW} of from −0.5 to +1.5 and a dielectric constant εᵣ at 20° C. of from 5.0 to 30 and at least one non-polar organic solvent having a dielectric constant εᵣ at 20° C. of from ≤3.0 and an n-octanol-water partition coefficient log K_{OW} of ≥3.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
wherein the microcrystals of the at least one limus active agent have a crystal size in the range of 1 μm to 300 μm,
   wherein the at least one limus active agent is selected from rapamycin (sirolimus) and everolimus,
   wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent mixture, in which the at least one tri-O-acylglycerol dissolves and in which the microcrystals of the at least one limus active agent do not dissolve,
wherein the microcrystals of the at least one limus active agent have a crystal size in the range of 1 μm to 300 μm,
   wherein the at least one limus active agent is selected from rapamycin (sirolimus) and everolimus,
   wherein the solvent mixture is a mixture of at least one polar organic solvent having an n-octanol-water partition coefficient log K_{OW} of from −0.5 to +1.5 and a dielectric constant εᵣ at 20° C. of from 5.0 to 30 and at least one non-polar organic solvent having a dielectric constant εᵣ at 20° C. of from ≤3.0 and an n-octanol-water partition coefficient log K_{OW} of ≥3.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent mixture, in which the at least one tri-O-acylglycerol dissolves and in which the microcrystals of the at least one limus active agent do not dissolve,
wherein the at least one limus active agent has a crystallinity of at least 90% by weight,
   wherein the at least one limus active agent is selected from rapamycin (sirolimus) and everolimus,
   wherein the solvent mixture is selected from ethanol and cyclohexane or ethyl acetate and heptane.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
wherein the microcrystals of the at least one limus active agent have a crystal size in the range of 1 μm to 300 μm,
   wherein the at least one limus active agent has a crystallinity of at least 90% by weight,
   wherein the at least one limus active agent is selected from rapamycin (sirolimus), and everolimus,
   wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent mixture, in which the at least one tri-O-acylglycerol dissolves and in which the microcrystals of the at least one limus active agent do not dissolve,
wherein the microcrystals of the at least one limus active agent have a crystal size in the range of 1 μm to 300 μm,
   wherein the at least one limus active agent has a crystallinity of at least 90% by weight,
   wherein the at least one limus active agent is selected from rapamycin (sirolimus) and everolimus,
   wherein the solvent mixture is a mixture of at least one polar organic solvent having an n-octanol-water partition coefficient log K_{OW} of from −0.5 to +1.5 and a dielectric constant εᵣ at 20° C. of from 5.0 to 30 and at least one non-polar organic solvent having a dielectric constant εᵣ at 20° C. of from ≤3.0 and an n-octanol-water partition coefficient log K_{OW} of ≥3.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
wherein the at least one tri-O-acylglycerol and the at least one limus active agent are present in a mass ratio of 10%-30% tri-O-acylglycerol to 90%-70% limus active agent,
   wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent mixture, in which the at least one tri-O-acylglycerol dissolves and in which the microcrystals of the at least one limus active agent do not dissolve,
wherein the at least one tri-O-acylglycerol and the at least one limus active agent are present in a mass ratio of 10%-30% tri-O-acylglycerol to 90%-70% limus active agent,
   wherein the solvent mixture is a mixture of at least one polar organic solvent having an n-octanol-water partition coefficient log K_{OW} of from −0.5 to +1.5 and a dielectric constant εᵣ at 20° C. of from 5.0 to 30 and at least one non-polar organic solvent having a dielectric constant εᵣ at 20° C. of from ≤3.0 and an n-octanol-water partition coefficient log K_{OW} of ≥3.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
wherein the microcrystals of the at least one limus active agent have a crystal size in the range of 1 μm to 300 μm,
   wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus,
   wherein the at least one tri-O-acylglycerol and the at least one limus active agent are present in a mass ratio of 10%-30% tri-O-acylglycerol to 90%-70% limus active agent,
   wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent mixture, in which the at least one tri-O-acylglycerol dissolves and in which the microcrystals of the at least one limus active agent do not dissolve,
wherein the microcrystals of the at least one limus active agent have a crystal size in the range of 1 μm to 300 μm,
   wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus,
   wherein the at least one tri-O-acylglycerol and the at least one limus active agent are present in a mass ratio of 10%-30% tri-O-acylglycerol to 90%-70% limus active agent,
   wherein the solvent mixture is a mixture of at least one polar organic solvent having an n-octanol-water partition coefficient log K_{OW} of from −0.5 to +1.5 and a dielectric constant εᵣ at 20° C. of from 5.0 to 30 and at least one non-polar organic solvent having a dielectric constant εᵣ at 20° C. of from ≤3.0 and an n-octanol-water partition coefficient log K_{OW} of ≥3.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
wherein the at least one limus active agent has a crystallinity of at least 90% by weight,
   wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus,
   wherein the at least one tri-O-acylglycerol and the at least one limus active agent are present in a mass ratio of 10%-30% tri-O-acylglycerol to 90%-70% limus active agent,
   wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent mixture, in which the at least one tri-O-acylglycerol dissolves and in which the microcrystals of the at least one limus active agent do not dissolve,
wherein the at least one limus active agent has a crystallinity of at least 90% by weight,
   wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus,
   wherein the at least one tri-O-acylglycerol and the at least one limus active agent are present in a mass ratio of 10%-30% tri-O-acylglycerol to 90%-70% limus active agent,
   wherein the solvent mixture is a mixture of at least one polar organic solvent having an n-octanol-water partition coefficient log K_{OW} of from −0.5 to +1.5 and a dielectric constant εᵣ at 20° C. of from 5.0 to 30 and at least one non-polar organic solvent having a dielectric constant εᵣ at 20° C. of from ≤3.0 and an n-octanol-water partition coefficient log K_{OW} of ≥3.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
wherein the microcrystals of the at least one limus active agent have a crystal size in the range of 1 μm to 300 μm,
   wherein the at least one limus active agent is selected from rapamycin (sirolimus), and everolimus,
   wherein the at least one tri-O-acylglycerol and the at least one limus active agent are present in a mass ratio of 10%-30% tri-O-acylglycerol to 90%-70% limus active agent,
   wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent mixture, in which the at least one tri-O-acylglycerol dissolves and in which the microcrystals of the at least one limus active agent do not dissolve,
wherein the microcrystals of the at least one limus active agent have a crystal size in the range of 1 μm to 300 μm,
   wherein the at least one limus active agent is selected from rapamycin (sirolimus) and everolimus,
   wherein the at least one tri-O-acylglycerol and the at least one limus active agent are present in a mass ratio of 10%-30% tri-O-acylglycerol to 90%-70% limus active agent,
   wherein the solvent mixture is a mixture of at least one polar organic solvent having an n-octanol-water partition coefficient log K_{OW} of from −0.5 to +1.5 and a dielectric constant εᵣ at 20° C. of from 5.0 to 30 and at least one non-polar organic solvent having a dielectric constant εᵣ at 20° C. of from ≤3.0 and an n-octanol-water partition coefficient log K_{OW} of ≥3.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve,
wherein the at least one limus active agent has a crystallinity of at least 90% by weight,
   wherein the at least one limus active agent is selected from rapamycin (sirolimus) and everolimus,
   wherein the at least one tri-O-acylglycerol and the at least one limus active agent are present in a mass ratio of 10%-30% tri-O-acylglycerol to 90%-70% limus active agent,
   wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

The present invention relates to a suspension for coating of a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent mixture, in which the at least one tri-O-acylglycerol dissolves and in which the microcrystals of the at least one limus active agent do not dissolve,
wherein the at least one limus active agent has a crystallinity of at least 90% by weight,
   wherein the at least one limus active agent is selected from rapamycin (sirolimus) and everolimus,
   wherein the at least one tri-O-acylglycerol and the at least one limus active agent are present in a mass ratio of 10%-30% tri-O-acylglycerol to 90%-70% limus active agent,
   wherein the solvent mixture is a mixture of at least one polar organic solvent having an n-octanol-water partition coefficient log K_{OW} of −0.5 to +1.5 and a dielectric constant εᵣ at 20° C. of 5.0 to 30 and at least one non-polar organic solvent having a dielectric constant εᵣ at 20° C. of ≤3.0 and an n-octanol-water partition coefficient log K_{OW} of ≥3.0.

Thus, the present invention preferably relates to a suspension for coating a medical device, preferably selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture, in which the at least one tri-O-acylglycerol dissolves and in which the microcrystals of the at least one limus active agent do not dissolve when the at least one tri-O-acylglycerol is present,
   wherein the suspension contains 1-6% limus active agent.

In some further embodiments, the coating suspension may consist only of the three ingredients a) at least one tri-O-acylglycerol selected from the group consisting of trioctanoylglycerol, trinonanoylglycerol, tridecanoylglycerol, and triundecanoylglycerol or a mixture of trioctanoylglycerol, trinonanoylglycerol, tridecanoylglycerol, and/or triundecanoylglycerol; b) at least one limus active agent in the form of microcrystals; and c) at least one solvent or a solvent mixture in which the at least one tri-O-acylglycerol dissolves and in which the microcrystals of the at least one limus active agent do not dissolve.

Additives

In addition to the above-mentioned at least one tri-O-acylglycerol selected from the group consisting of trioctanoylglycerol, trinonanoylglycerol, tridecanoylglycerol, and triundecanoylglycerol or a mixture of trioctanoylglycerol, trinonanoylglycerol, tridecanoylglycerol, and/or triundecanoylglycerol, the suspension according to the invention may also contain one or more additives. In particular, the one or more additives are preferably dissolved in the suspension.

In preferred embodiments, the suspension according to the invention does not contain polymers, oligomers, metals or metal particles, organometallic compounds and salts. In some embodiments, the medical device coating suspension according to the invention is therefore free of polymers, oligomers, metals or metal particles, organometallic compounds, and salts.

In preferred embodiments, an antioxidant may be present as an additive in the suspension according to the invention. An antioxidant may be added to the suspension for the purpose of preserving the at least one limus active agent.

Suitable antioxidants include butylhydroxyltoluene (BHT), butylhydroxyanisole, ascorbyl palmitate, ascorbyl stearate, tocopherol acetate, ascorbic acid, tocopherols and tocotrienols (e.g. alpha-tocopherol), carotenoids such as B-carotene, zeaxanthin, lycopene and lutein, vitamin C, nordihydroguaretic acid, probucol, propyl gallate, secondary plant compounds (flavonoids) such as catechin, gallocatechin, epicatechin, epigallocatechin gallate, taxifolin, isoliquiritigenin, xanthohumol, morin, quercetin (glycoside rutin and methyl ether isorhamnetin), kaempferol, myricetin, fisetin, aureusidin, luteolin, apigenin, hesperetin, naringenin, eriodictyol, genistein, daidzein, licoricidin anthocyanins, allicin, astaxanthin glutathione, resveratrol, derivatives therof and combinations thereof.

Preferred antioxidants are therefore butylated hydroxytoluene (BHT), which prevents or delays rancidity especially in fat phases after contact with air, butylated hydroxyanisole (BHA), tocopherols, carotenoids, flavonoids, and of course also mixtures of the antioxidants.

Butylhydroxytoluene (BHT) is particularly preferred as an antioxidant.

If one or more antioxidants are added to the suspension, their total content is calculated to be between 0.001-15.0 wt. % with respect to limus active agent, preferably 0.01-10.0 wt. % and particularly preferably 0.05-5.0 wt. %.

In some embodiments, a flocculation inhibitor may be present in the suspension of the invention as an additive that may prevent sedimentation of the microcrystals of the at least one limus active agent in the suspension. Suitable flocculation inhibitors include, but are not limited to, polysorbates such as Tween 80. Flocculation inhibitors are preferably used in the preparation of crystal suspensions at very low active agent levels.

For example, a 3% suspension containing everolimus with uniform distribution of the crystals can be prepared without a flocculation inhibitor; from about 1.5-1.0% suspension (w/v) and lower, the addition of flocculation inhibitors can be advantageous, as these additionally prevent sedimentation of the microcrystals and thus continue to allow uniform coating.

If one or more flocculation inhibitors are added to the suspension, the additional amount that can keep the microcrystals in suspension must be determined individually for the respective limus active agent. The total amount of microcrystalline limus active agent in the suspension is preferably very low between 1.0-0.001 wt. %, further preferred are 0.5-0.005 wt. %, and especially preferred are 0.1-0.01 wt. %.

It is also possible to add another non-polymeric excipient to the solution as a matrix. For example, contrast agents or contrast agent analogs are suitable, as are biocompatible organic substances that also improve or do not negatively change the coating properties.

In some embodiments, the suspension of the present invention may also contain one or more polymers as an additive, such as polyvinylpyrrolidone (PVP). Suitable polymer additives are polymers that can be dissolved in organic solvents, especially non-polar solvents. Very hydrophilic, water-soluble polymers that are hardly soluble or virtually insoluble in organic solvents are not preferred. In addition, care must be taken to ensure that the microcrystals of the at least one limus active agent do not become attached or dissolved in the presence of a polymer dissolved in the suspension. Polymers for coatings of medical devices are known from the prior art. The person skilled in the art is thus easily able to select a suitable polymer additive.

However, polymer-free suspensions for coating of medical devices are particularly preferred herein.

Thus, the present invention relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent.

Suitable additives are the substances mentioned below, preferably antioxidants, polyvinylpyrrolidone (PVP) and flocculation inhibitors.

Thus, the present invention relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the microcrystals of the at least one limus active agent have a crystal size ranging from 1 μm to 300 μm.

Thus, the present invention relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the at least one limus active agent has a crystallinity of at least 90% by weight.

Thus, the present invention relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the at least one limus active agent is selected from rapamycin (sirolimus), and and everolimus.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the microcrystals of the at least one limus active agent have a crystal size ranging from 1 μm to 300 μm,
wherein the at least one limus active agent is selected from rapamycin (sirolimus), and everolimus.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the at least one limus active agent has a crystallinity of at least 90% by weight,
wherein the at least one limus active agent is selected from rapamycin (sirolimus) and everolimus.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the microcrystals of the at least one limus active agent have a crystal size ranging from 1 μm to 300 μm,
wherein the at least one limus active agent has a crystallinity of at least 90% by weight,
wherein the at least one limus active agent is selected from rapamycin (sirolimus) and everolimus.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the at least one tri-O-acylglycerol and the at least one limus active agent are present at a mass fraction of 10%-30% tri-O-acylglycerol to 90%-70% limus active agent.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the microcrystals of the at least one limus active agent have a crystal size ranging from 1 μm to 300 μm,
wherein the at least one limus active agent is selected from rapamycin (sirolimus) and everolimus,
wherein the at least one tri-O-acylglycerol and the at least one limus active agent are present at a mass fraction of 10%-30% tri-O-acylglycerol to 90%-70% limus active agent.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the at least one limus active agent has a crystallinity of at least 90% by weight,
wherein the at least one limus active agent is selected from rapamycin (sirolimus) and everolimus,
wherein the at least one tri-O-acylglycerol and the at least one limus active agent are present at a mass fraction of 10%-30% tri-O-acylglycerol to 90%-70% limus active agent.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the microcrystals of the at least one limus active agent have a crystal size ranging from 1 μm to 300 μm,
wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the microcrystals of the at least one limus active agent have a crystal size ranging from 1 μm to 300 μm,
wherein the solvent mixture is a mixture of at least one polar organic solvent having an n-octanol-water partition coefficient log K_{OW} of −0.5 to +1.5 and a dielectric constant εᵣ at 20° C. of 5.0 to 30 and at least one non-polar organic solvent having a dielectric constant εᵣ at 20° C. of ≤3.0 and an n-octanol-water partition coefficient log K_{OW} of ≥3.0.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the at least one limus active agent is selected from rapamycin (sirolimus) and everolimus,
wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the at least one limus active agent is selected from rapamycin (sirolimus) and everolimus,
wherein the solvent mixture is a mixture of at least one polar organic solvent having an n-octanol-water partition coefficient log K_{OW} of −0.5 to +1.5 and a dielectric constant εᵣ at 20° C. of 5.0 to 30 and at least one non-polar organic solvent having a dielectric constant εᵣ at 20° C. of ≤3.0 and an n-octanol-water partition coefficient log K_{OW} of ≥3.0.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the microcrystals of the at least one limus active agent have a crystal size ranging from 1 μm to 300 μm,
wherein the at least one limus active agent is selected from rapamycin (sirolimus) and everolimus,
wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the microcrystals of the at least one limus active agent have a crystal size ranging from 1 μm to 300 μm,
wherein the at least one limus active agent is selected from rapamycin (sirolimus) and everolimus,
wherein the solvent mixture is a mixture of at least one polar organic solvent having an n-octanol-water partition coefficient log K_{OW} of −0.5 to +1.5 and a dielectric constant εᵣ at 20° C. of 5.0 to 30 and at least one non-polar organic solvent having a dielectric constant εᵣ at 20° C. of ≤3.0 and an n-octanol-water partition coefficient log K_{OW} of ≥3.0.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the at least one limus active agent has a crystallinity of at least 90% by weight,
wherein the at least one limus active agent is selected from rapamycin (sirolimus) and everolimus,
wherein the solvent mixture is selected from ethanol and cyclohexane or ethyl acetate and heptane.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the microcrystals of the at least one limus active agent have a crystal size ranging from 1 μm to 300 μm,
wherein the at least one limus active agent has a crystallinity of at least 90% by weight,
wherein the at least one limus active agent is selected from rapamycin (sirolimus) and everolimus,
wherein the solvent is a non-solvent having a dielectric constant εᵣ at 20° C. of ≤2.0, or the solvent mixture contains at least 50% by volume of a non-solvent having a dielectric constant εᵣ at 20° C. of ≤2.0.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the microcrystals of the at least one limus active agent have a crystal size ranging from 1 μm to 300 μm,
wherein the at least one limus active agent has a crystallinity of at least 90% by weight,
wherein the at least one limus active agent is selected from rapamycin (sirolimus) and everolimus,
wherein the solvent mixture is a mixture of at least one polar organic solvent having an n-octanol-water partition coefficient log K_{OW} of −0.5 to +1.5 and a dielectric constant εᵣ at 20° C. of 5.0 to 30 and at least one non-polar organic solvent having a dielectric constant εᵣ at 20° C. of ≤3.0 and an n-octanol-water partition coefficient log K_{OW} of ≥3.0.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the at least one tri-O-acylglycerol and the at least one limus active agent are present at a mass fraction of 10%-30% tri-O-acylglycerol to 90%-70% limus active agent,
wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the at least one tri-O-acylglycerol and the at least one limus active agent are present at a mass fraction of 10%-30% tri-O-acylglycerol to 90%-70% limus active agent,
wherein the solvent mixture is a mixture of at least one polar organic solvent having an n-octanol-water partition coefficient log K_{OW} of −0.5 to +1.5 and a dielectric constant εᵣ at 20° C. of 5.0 to 30 and at least one non-polar organic solvent having a dielectric constant εᵣ at 20° C. of ≤3.0 and an n-octanol-water partition coefficient log K_{OW} of ≥3.0.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the microcrystals of the at least one limus active agent have a crystal size ranging from 1 μm to 300 μm,
wherein the at least one limus active agent is selected from rapamycin (sirolimus) and everolimus,
wherein the at least one tri-O-acylglycerol and the at least one limus active agent are present at a mass fraction of 10%-30% tri-O-acylglycerol to 90%-70% limus active agent,
wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the microcrystals of the at least one limus active agent have a crystal size ranging from 1 μm to 300 μm,
wherein the at least one limus active agent is selected from rapamycin (sirolimus) and everolimus,
wherein the at least one tri-O-acylglycerol and the at least one limus active agent are present at a mass fraction of 10%-30% tri-O-acylglycerol to 90%-70% limus active agent,
wherein the solvent mixture is a mixture of at least one polar organic solvent having an n-octanol-water partition coefficient log K_{OW} of −0.5 to +1.5 and a dielectric constant εᵣ at 20° C. of 5.0 to 30 and at least one non-polar organic solvent having a dielectric constant εᵣ at 20° C. of ≤3.0 and an n-octanol-water partition coefficient log K_{OW} of ≥3.0.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the at least one limus active agent has a crystallinity of at least 90% by weight,
wherein the at least one limus active agent is selected from rapamycin (sirolimus) and everolimus,
wherein the at least one tri-O-acylglycerol and the at least one limus active agent are present at a mass fraction of 10%-30% tri-O-acylglycerol to 90%-70% limus active agent,
wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

Thus, the present invention preferably relates to a suspension for coating of a medical device, preferably selected from a catheter balloon, a balloon catheter, a stent or a cannula, the suspension consisting of:
(a) at least one tri-O-acylglycerol selected from the group consisting of trioctanoylglycerol, trinonanoylglycerol, tridecanoylglycerol, and triundecanoylglycerol; and
b) at least one limus active agent in the form of microcrystals, wherein the limus active agent is selected from the group consisting of rapamycin (sirolimus), everolimus, zotarolimus, umirolimus, deforolimus, myolimus, novolimus, pimecrolimus, ridaforolimus, tacrolimus and temsirolimus, and
c) a solvent or a solvent mixture in which the at least one tri-O-acylglycerol dissolves and in which the microcrystals of the at least one limus active agent do not dissolve when the at least one tri-O-acylglycerol is present, and
d) up to 5.0% by weight, based on the limus active agent, of additives or up to 15.0% by weight, based on the limus active agent, of antioxidants as additives and up to 5.0% by weight, based on the limus active agent, of additives which are not antioxidants, wherein the total amount of additives does not exceed 15.0% by weight, based on the limus active agent,
wherein the at least one limus active agent has a crystallinity of at least 90% by weight,
wherein the at least one limus active agent is selected from rapamycin (sirolimus) and everolimus,
wherein the at least one tri-O-acylglycerol and the at least one limus active agent are present at a mass fraction of 10%-30% tri-O-acylglycerol to 90%-70% limus active agent,
wherein the solvent mixture is a mixture of at least one polar organic solvent having an n-octanol-water partition coefficient log K_{OW} of −0.5 to +1.5 and a dielectric constant εᵣ at 20° C. of 5.0 to 30 and at least one non-polar organic solvent having a dielectric constant εᵣ at 20° C. of ≤3.0 and an n-octanol-water partition coefficient log K_{OW} of ≥3.0.

Method for Preparing the Suspension

The present invention further relates to a method for preparing a suspension for coating a medical device, preferably selected from a catheter balloon, a balloon catheter, a stent, or a cannula, comprising the following steps:
a) dissolving at least one tri-O-acylglycerol selected from the group consisting of trioctanoylglycerol, trinonanoylglycerol, tridecanoylglycerol, and triundecanoylglycerol in a solvent or solvent mixture;
b) addition of at least one limus active agent in the form of microcrystals to the solution of step a) or addition of the solution of step a) to at least one limus active agent in the form of microcrystals,
wherein the microcrystals of the at least one limus active agent do not dissolve in the solution of step a).

In other words, the present invention further relates to a method for preparing a suspension for coating of a medical device, preferably selected from a catheter balloon, a balloon catheter, a stent, or a cannula, comprising the following steps:
a) dissolving at least one tri-O-acylglycerol selected from the group consisting of trioctanoylglycerol, trinonanoylglycerol, tridecanoylglycerol, and triundecanoylglycerol in a solvent or solvent mixture;
b) preparing a suspension of at least one limus active agent in the form of microcrystals and the solution of step a),
wherein the microcrystals of the at least one limus active agent do not dissolve in the solution of step a).

Preferably, the at least one limus active agent has a crystallinity of at least 90% by weight. Preferably, the microcrystals of the at least one limus active agent have a crystal size in the range of 1 μm to 300 μm, more preferably a crystal size of at most 100 μm, more preferably a crystal size in the range of 10 μm to 50 μm. More preferably, the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus. Even more preferably, the at least one limus active agent is selected from rapamycin (sirolimus) and everolimus. More preferably, the at least one limus active agent is everolimus. Preferably, the solvent is a non-solvent having a dielectric constant εᵣ at 20° C. of ≤2.0 or the solvent mixture contains at least 50% by vol. % of a non-solvent having a dielectric constant εᵣ at 20° C. of ≤2.0. Preferably, the solvent mixture is a mixture of at least one polar organic solvent with an n-octanol-water partition coefficient log K_{OW} of −0.5 to +1.5 and a dielectric constant εᵣ at 20° C. of 5.0 to 30, and at least one nonpolar organic solvent having a dielectric constant εᵣ at 20° C. of from ≤3.0 and an n-octanol water partition coefficient log K_{OW} of ≥3.0.

With other words, the present invention relates to a method for preparing a suspension for coating of a medical device, preferably selected from a catheter balloon, a balloon catheter, a stent, or a cannula, comprising the following steps:
(a) providing a solution of at least one tri-O-acylglycerol selected from the group consisting of trioctanoylglycerol, trinonanoylglycerol, tridecanoylglycerol, and triundecanoylglycerol in a solvent or a solvent mixture;
b) providing at least one limus active agent in the form of microcrystals,
c) preparing a suspension by combining the solution according to step a) and the at least one limus active agent in the form of microcrystals according to step b),
wherein the microcrystals of the at least one limus active agent according to step b) do not dissolve in the solution according to step a).

Preferably, the at least one limus active agent has a crystallinity of at least 90% by weight. Preferably, the microcrystals of the at least one limus active agent have a crystal size in the range of 1 μm to 300 μm, more preferably a crystal size of at most 100 μm, more preferably a crystal size in the range of 10 μm to 50 μm. More preferably, the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus. Even more preferably, the at least one limus active agent is selected from rapamycin (sirolimus) and everolimus. More preferably, the at least one limus active agent is everolimus. Preferably, the solvent is a non-solvent having a dielectric constant εᵣ at 20° C. of ≤2.0 or the solvent mixture contains at least 50% by vol. % of a non-solvent having a dielectric constant εᵣ at 20° C. of ≤2.0. Preferably, the solvent mixture is a mixture of at least one polar organic solvent with an n-octanol-water partition coefficient log K_{OW} of −0.5 to +1.5 and a dielectric constant εᵣ at 20° C. of 5.0 to 30, and at least one nonpolar organic solvent having a dielectric constant εᵣ at 20° C. of from ≤3.0 and an n-octanol water partition coefficient log K_{OW} of ≥3.0.

It has proven essential to provide the at least one limus active agent in the form of microcrystals and to add the at least one limus active agent in the form of microcrystals together with the solution of the at least one tri-O-acylglycerol in the solvent or solvent mixture so that a stable crystal suspension is formed. If, on the other hand, the microcrystals of the limus active agent are first added to the solvent or solvent mixture and only then the at least one tri-O-acylglycerol is added, no suspension of the microcrystals of the limus active agent according to the invention is formed, from which firmly adhering coatings on medical devices can be produced.

The sequence of steps for preparing the crystal suspension of the limus active agent is therefore essential and cannot be interchanged. Similarly, unsuitable coatings are formed when an attempt is made to produce limus active agent crystals from a solution of the limus active agent in a solvent or solvent mixture containing the at least one tri-O-acylglycerol after coating on the surface of the medical device.

The present invention also relates to a method for preparing a suspension for coating of a medical device, preferably selected from a catheter balloon, a balloon catheter, a stent, or a cannula, comprising the following steps:
a′) dissolving at least one tri-O-acylglycerol selected from the group consisting of trioctanoylglycerol, trinonanoylglycerol, tridecanoylglycerol, and triundecanoylglycerol in a solvent, preferably in a polar organic solvent,
a″) adding a non-polar organic solvent, preferably a non-solvent, to the solution from step a′); and optionally homogenizing and filtering,
b) addition of at least one limus active agent in the form of microcrystals to the solution from step a″) or addition of the solution from step a″) to at least one limus active agent in the form of microcrystals,
wherein the microcrystals of the at least one limus active agent are not soluble in the solution of step a″).

The present invention also relates to a method for preparing a suspension for coating of a medical device, preferably selected from a catheter balloon, a catheter balloon, a balloon catheter, a stent, or a cannula, comprising the following steps:
a′) dissolving at least one tri-O-acylglycerol selected from the group consisting of trioctanoylglycerol, trinonanoylglycerol, tridecanoylglycerol, and triundecanoylglycerol in a solvent, preferably in a polar organic solvent,
a″) adding a non-polar organic solvent, preferably a non-solvent, to the solution from step a′); and optionally homogenizing and filtering,
b) providing at least one limus active agent in the form of microcrystals,
c) preparing a suspension by combining the solution according to step a″) and the at least one limus active agent in the form of microcrystals according to step b),
   wherein the microcrystals of the at least one limus active agent according to step b) do not dissolve in the solution according to step a″).

Preferably, the at least one limus active agent has a crystallinity of at least 90% by weight. Preferably, the microcrystals of the at least one limus active agent have a crystal size in the range of 1 μm to 300 μm, more preferably a crystal size of at most 100 μm, more preferably a crystal size in the range of 10 μm to 50 μm. More preferably, the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus. Even more preferably, the at least one limus active agent is selected from rapamycin (sirolimus) and everolimus. More preferably, the at least one limus active agent is everolimus. Preferably, the non-solvent has a dielectric constant εᵣ at 20° C. of ≤2.0. Preferably, the solution contains at least 50 vol. % non-solvent having a dielectric constant εᵣ at 20° C. of ≤2.0. Preferably, the solvent mixture is a mixture of at least one polar organic solvent with an n-octanol-water partition coefficient log K_{OW} of −0.5 to +1.5 and a dielectric constant εᵣ at 20° C. of 5.0 to 30, and at least one nonpolar organic solvent having a dielectric constant εᵣ at 20° C. of from ≤3.0 and an n-octanol water partition coefficient log K_{OW} of ≥3.0.

Coating Method

The present invention also relates to a method of coating of a medical device, preferably selected from a catheter balloon, a balloon catheter, a stent, or a cannula, with a suspension, comprising the following steps:
a) providing a medical device with a medical device surface,
b) providing a suspension comprising at least one tri-O-acylglycerol selected from the group consisting of trioctanoylglycerol, trinonanoylglycerol, tridecanoylglycerol, and triundecanoylglycerol, at least one limus active agent in the form of microcrystals, and a solvent or a solvent mixture in which the at least one tri-O-acylglycerol dissolves and in which the microcrystals of the at least one limus active agent do not dissolve or do not dissolve when the at least one tri-O-acylglycerol is present; and
c) applying the suspension to the medical device surface by means of a syringe method, pipetting method, capillary method, fold spraying method, dipping method, spraying method, dragging method, thread dragging method, drop dragging method or rolling method.

The present invention also relates to a method of coating of a medical device, preferably selected from a catheter balloon, a balloon catheter, a stent, or a cannula, with a suspension, comprising the following steps:
a) providing a medical device with a medical device surface, optionally with a pre-treated surface (conditioning of the surface), wherein the medical device surface is uncoated or coated;
b) providing a suspension comprising at least one tri-O-acylglycerol selected from the group consisting of trioctanoylglycerol, trinonanoylglycerol, tridecanoylglycerol, and triundecanoylglycerol, at least one limus active agent in the form of microcrystals, and a solvent or a solvent mixture in which the at least one tri-O-acylglycerol dissolves and in which the microcrystals of the at least one limus active agent do not dissolve or do not dissolve when the at least one tri-O-acylglycerol is present; and
c) applying the suspension to the surface of the medical device by means of a syringe method, pipetting method, capillary method, fold spray method, dipping method, spraying method, dragging method, thread dragging method, drop dragging method or rolling method.

In preferred embodiments, the method further comprises a step d) drying the coating after step c).

Preferred herein are special coating methods for coating of medical devices in which the medical device can be coated with a defined amount of microcrystalline limus active agent, wherein in said coating methods a coating device with a volume measuring device for the targeted delivery of a defined amount of the coating suspension according to the invention onto the medical device surface by means of a dispensing device is preferably used.

Any device capable of providing a defined amount of coating suspension or measuring or indicating the dispensed amount of coating suspension can serve as a volume measuring device. Volume measuring devices are therefore in the simplest case scales, scaled pipettes, scaled burettes, scaled containers, scaled cavities as well as pumps, valves, syringes or other piston-shaped containers, which are capable of providing or conveying or dispensing a defined quantity of coating suspension. Thus, the volume measuring device serves to either provide or dispense a defined amount of a coating suspension or to measure and/or display a dispensed amount of coating suspension. Thus, the volume measuring device serves to determine or measure the amount of coating suspension and thus of microcrystalline limus active agent transferred from the dispensing device to the medical device surface.

The most important aspect of the coating device, however, is the dispensing device, which can be designed as a nozzle, a plurality of nozzles, a thread, a network of threads, a piece of textile, a leather strip, a sponge, a ball, a syringe, a needle, a cannula or a capillary. Depending on the design of the dispensing device, slightly modified coating methods result, all of which are based on the basic principle of transferring a measurable or defined amount of microcrystalline limus active agent to the surface of the medical device without loss. In this way, a coating with a defined active agent concentration or active agent amount of microcrystalline limus active agent and thus a reproducible coating is provided. Various terms are used herein to distinguish the methods, namely, syringe method, pipetting method, capillary method, fold spray method, dipping method, spraying method, dragging method, thread dragging method, droplet dragging method, or rolling method, which are the preferred embodiments of the present invention.

As a particularly preferred coating method of medical devices with crystal suspensions, the droplet dosing technique using the microdosing method such as the pipetting method or droplet dragging method is used herein. These can be used to obtain particularly uniform coatings with likewise uniform active agent concentration of the microcrystalline limus active agent on the medical device surface, as long as it is ensured that the microcrystals of the limus active agent remain uniformly distributed.

The studies on the recovery rate of active agent on balloon catheters divided into equal segments confirm the uniformity of the coating and thus the success of using a crystal suspension (see example 7) as well as a 100% recovery rate.

In addition, to ensure uniform distribution of the crystals, a pneumatic swivel can be used to agitate the suspension during the coating process to prevent possible sedimentation, which can be advantageous as a precautionary measure for crystal contents of less than 2% (w/v).

On the other hand, for any pretreatment of the medical device surface, e.g. conditioning or applying a base coat, the other common coating methods such as spraying, dipping, brushing, pipetting, drop dragging, rolling, spinning, in situ deposition, screen printing, vapor deposition or spraying can also be used. The above methods can also be combined.

Coated Medical Device

The suspension of the present invention is particularly suitable for providing coated medical devices having an active agent-releasing coating comprising at least one tri-O-acylglycerol selected from the group consisting of trioctanoylglycerol, trinonanoylglycerol, tridecanoylglycerol, and triundecanoylglycerol, and at least one limus active agent in the form of microcrystals.

The present invention therefore also relates to coated medical devices, in particular medical devices selected from a catheter balloon, a balloon catheter, a stent, or a cannula, having a coating comprising at least one tri-O-acylglycerol selected from the group consisting of trioctanoylglycerol, trinonanoylglycerol, tridecanoylglycerol, and triundecanoylglycerol, and at least one limus active agent in the form of microcrystals.

The present invention therefore further relates to coated medical devices, in particular medical devices selected from a catheter balloon, a balloon catheter, a stent, or a cannula, having a coating consisting of at least one tri-O-acylglycerol selected from the group consisting of trioctanoylglycerol, trinonanoylglycerol, tridecanoylglycerol, and triundecanoylglycerol, and at least one limus active agent in the form of microcrystals.

The term “coating” is intended to include not only a coating on the medical device surface but also a filling or coating of folds, cavities, pores, microneedles or other fillable spaces on or between or within the material as well as, in the case of expandable, folded or collapsed medical devices, deflated, partially inflated and fully inflated or unfolded or partially unfolded medical devices.

The term “on the medical device surface”, as used herein, preferably means that an application is made directly to the medical device surface, i.e., directly to the material of the medical device. For example, if the medical device is made of polyamide, this means that the application is made to the polyamide from which the medical device is made. If the medical device is made of, for example, polyamide and is subsequently coated with a polymer, then the application would not be made on the medical device surface.

It is preferred if the entire medical device surface is uniformly coated. Furthermore, it is preferred if there is a uniform distribution of the microcrystalline limus active agent on the medical device surface. However, the surface can also be only partially coated or coated differently at different points (e.g., different coating thicknesses, different coatings, different active agent concentrations, only selected delimited areas, etc.).

The term “medical device”, as used herein, refers to articles or substances that serve to detect, prevent, monitor, treat or alleviate diseases, but achieve this purpose primarily (“intended primary effect”) by physical means rather than by pharmacological/immunological means or by metabolic action. However, the physical effect of medical devices may well be supported by pharmacological, immunological or metabolic effects. Medical devices can be divided into medical devices for long-term use and medical devices for short-term use, depending on whether the medical device has short-term or long-term contact with the organism. All medical devices that are intended to remain in the body are considered to be for long-term use. Medical devices with less long-term to very short-term use are medical devices that can be removed after a certain period of time and are used for a limited period of time.

Examples of long-term medical devices include, but are not limited to, non-biodegradable, biostable stents, implants, joint implants, vascular prostheses, brain pacemakers (such as those used in used for Parkinson's disease), artificial hearts, port catheters, vision implants, ocular lens replacements, retinal replacements, vitreous replacements, corneas, dental implants, cochlear implants, reconstructive implants, cranial reconstructions, bone replacements, penile prostheses, sphincter prostheses and the like.

Examples of less long-term to very short-term medical devices include, but are not limited to, all forms and types of catheters, balloon catheters, angioplasty catheters, bladder catheters, breathing tubes, venous catheters, cannulas of all types, needles, winged cannulas (butterflies), drug depots, fixations, e.g., for surgical treatment of bone fractures, artificial accesses, tubes, sutures, staples, and the like.

The term “balloon” or “catheter balloon” generally refers to any expandable and re-compressible as well as temporarily implantable medical device, which is generally used in conjunction with a catheter. The term “catheter balloon” as used herein refers to the dilatable portion, i.e., the balloon of a balloon catheter. “Balloon catheter” refers to a dilatation balloon catheter. Balloon catheter is a medical term for catheters that have a balloon attached to them. Examples of balloon catheters include, but are not limited to angioplasty balloon catheters used in percutaneous transluminal angioplasty to dilate and open narrowed or occluded blood vessels, bladder catheters, thrombectomy catheters used in treatment in vascular surgery, neuroradiology, and cardiology in the treatment of embolized and secondarily thrombosed peripheral arteries, but also used in neurothrombectomy in stroke therapy, embolectomy catheters used in vascular surgery for removal of fresh and soft emboli in the peripheral arterial system, Fogarty catheters, double balloon catheters, balloon catheters used in pneumology, micro-balloon catheters.

In some embodiments of the present invention, the medical device is selected from the group comprising or consisting of a catheter balloon, a balloon catheter, an angioplasty catheter, a bladder catheter, a stent, an implant, a joint implant, a vascular prosthesis, a port catheter, a visual prosthesis, an ocular implant, a dental implant, a cochlear implant, a reconstructive implant, a penile prosthesis, a sphincter prosthesis, a cardiac pacemaker, a brain pacemaker, a breathing tube, a venous catheter, a cannula, a needle, a winged cannula (butterfly), an artificial access, a tube, sutures, and a medical staple.

In particularly preferred embodiments of the present invention, the medical device is selected from the group comprising or consisting of a catheter balloon, a balloon catheter, a stent, or a cannula. Thus, in these embodiments, the medical device is preferably selected from the group comprising or consisting of a catheter balloon, a balloon catheter, an angioplasty catheter, a bladder catheter, a port catheter, a vein catheter, a peripheral catheter, a coronary catheter, an embolectomy catheter, a thrombectomy catheter, a neurothrombectomy catheter, a stent, a bioresorbable stent, a cannula, a hypodermic needle, a winged (butterfly) cannula, a peripheral vein indwelling cannula, and an epidural cannula. Further preferably, the medical device is selected from the group comprising or consisting of a catheter balloon, a balloon catheter, an angioplasty catheter, and a stent. Still further preferably, the medical device is selected from the group comprising or consisting of a catheter balloon, a balloon catheter, and an angioplasty catheter. More preferably, the medical device is a catheter balloon.

Catheter balloons, regardless of their field of application, can be made of common biocompatible flexible materials, in particular polymers as described further below and in particular polyamide, such as PA 12, polyester, polyurethanes, polyacrylates, polyethers, Pebax, etc. but also of combinations of suitable polymers, e.g. of superimposed layers of these materials, as well as of copolymers of these materials, blends and combinations of the embodiments of layers and copolymers and their blends.

An implant can be made of common biocompatible materials such as medical stainless steel, titanium, chromium, vanadium, tungsten, molybdenum, gold, iron, nitinol, magnesium, iron, zinc, alloys of the aforementioned metals, ceramics as well as polymeric biostable or bioresorbable material such as e.g. PTFE, polysulfones, polyvinylpyrrolidone, polyamide, e.g. PA 12, polyester, polyurethane, polyacrylates, polyethers, silicone, PMMA, combinations thereof, etc. The materials are either bioinert, biostable and/or biodegradable, and the implant is expandable, compressible or non-shape-changing.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the microcrystals of the at least one limus active agent have a crystal size in the range of 1 μm to 300 μm.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the at least one limus active agent has a crystallinity of at least 90% by weight.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the at least one limus active agent is selected from rapamycin (sirolimus) and everolimus.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the microcrystals of the at least one limus active agent have a crystal size in the range of 1 μm to 300 μm,
wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the at least one limus active agent has a crystallinity of at least 90% by weight,
wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus and temsirolimus.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the microcrystals of the at least one limus active agent have a crystal size in the range of 1 μm to 300 μm,
wherein the at least one limus active agent is selected from rapamycin (sirolimus) and everolimus.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the at least one limus active agent has a crystallinity of at least 90% by weight,
wherein the at least one limus active agent is selected from rapamycin (sirolimus) and everolimus.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the microcrystals of the at least one limus active agent have a crystal size in the range of 1 μm to 300 μm,
wherein the at least one limus active agent has a crystallinity of at least 90% by weight,
   wherein the at least one limus active agent is selected from rapamycin (sirolimus) and everolimus.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the at least one tri-O-acylglycerol and the at least one limus active agent are present in a mass ratio of 10%-30% tri-O-acylglycerol to 90%-70% limus active agent.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the microcrystals of the at least one limus active agent have a crystal size in the range of 1 μm to 300 μm,
wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus,
wherein the at least one tri-O-acylglycerol and the at least one limus active agent are present in a mass ratio of 10%-30% tri-O-acylglycerol to 90%-70% limus active agent.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the at least one limus active agent has a crystallinity of at least 90% by weight,
wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus,
wherein the at least one tri-O-acylglycerol and the at least one limus active agent are present in a mass ratio of 10%-30% tri-O-acylglycerol to 90%-70% limus active agent.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the microcrystals of the at least one limus active agent have a crystal size in the range of 1 μm to 300 μm,
wherein the at least one limus active agent is selected from rapamycin (sirolimus) and everolimus,
wherein the at least one tri-O-acylglycerol and the at least one limus active agent are present in a mass ratio of 10%-30% tri-O-acylglycerol to 90%-70% limus active agent.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the at least one limus active agent has a crystallinity of at least 90% by weight,
wherein the at least one limus active agent is selected from rapamycin (sirolimus) and everolimus,
wherein the at least one tri-O-acylglycerol and the at least one limus active agent are present in a mass ratio of 10%-30% tri-O-acylglycerol to 90%-70% limus active agent.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the microcrystals of the at least one limus active agent have a crystal size in the range of 1 μm to 300 μm,
wherein at least 70% of the limus active agent is in the form of microcrystals having a crystal size in the range of 10 μm to 50 μm.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the at least one limus active agent has a crystallinity of at least 90% by weight,
wherein at least 70% of the limus active agent is in the form of microcrystals having a crystal size in the range of 10 μm to 50 μm.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus and temsirolimus,
wherein at least 70% of the limus active agent is in the form of microcrystals having a crystal size in the range of 10 μm to 50 μm.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the at least one limus active agent is selected from rapamycin (sirolimus) and everolimus,
wherein at least 70% of the limus active agent is in the form of microcrystals having a crystal size in the range of 10 μm to 50 μm.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the microcrystals of the at least one limus active agent have a crystal size in the range of 1 μm to 300 μm,
wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, temsirolimus, and zotarolimus,
wherein at least 70% of the limus active agent is in the form of microcrystals having a crystal size in the range of 10 μm to 50 μm.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the at least one limus active agent has a crystallinity of at least 90% by weight,
wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus,
wherein at least 70% of the limus active agent is in the form of microcrystals having a crystal size in the range of 10 μm to 50 μm.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the microcrystals of the at least one limus active agent have a crystal size in the range of 1 μm to 300 μm,
wherein the at least one limus active agent is selected from rapamycin (sirolimus) and everolimus,
wherein at least 70% of the limus active agent is in the form of microcrystals having a crystal size in the range of 10 μm to 50 μm.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the at least one limus active agent has a crystallinity of at least 90% by weight,
wherein the at least one limus active agent is selected from rapamycin (sirolimus) and everolimus,
wherein at least 70% of the limus active agent is in the form of microcrystals having a crystal size in the range of 10 μm to 50 μm.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the microcrystals of the at least one limus active agent have a crystal size in the range of 1 μm to 300 μm,
wherein the at least one limus active agent has a crystallinity of at least 90% by weight,
wherein the at least one limus active agent is selected from rapamycin (sirolimus) and everolimus,
wherein at least 70% of the limus active agent is in the form of microcrystals having a crystal size in the range of 10 μm to 50 μm.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the at least one tri-O-acylglycerol and the at least one limus active agent are present in a mass ratio of 10%-30% tri-O-acylglycerol to 90%-70% limus active agent,
wherein at least 70% of the limus active agent is in the form of microcrystals having a crystal size in the range of 10 μm to 50 μm.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the microcrystals of the at least one limus active agent have a crystal size in the range of 1 μm to 300 μm,
wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus,
wherein the at least one tri-O-acylglycerol and the at least one limus active agent are present in a mass ratio of 10%-30% tri-O-acylglycerol to 90%-70% limus active agent,
wherein at least 70% of the limus active agent is in the form of microcrystals having a crystal size in the range of 10 μm to 50 μm.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the at least one limus active agent has a crystallinity of at least 90% by weight,
wherein the at least one limus active agent is selected from the group comprising or consisting of rapamycin (sirolimus), everolimus, zotarolimus, umirolimus, deforolimus, myolimus, novolimus, pimecrolimus, ridaforolimus, tacrolimus, and temsirolimus,
wherein the at least one tri-O-acylglycerol and the at least one limus active agent are present in a mass ratio of 10%-30% tri-O-acylglycerol to 90%-70% limus active agent,
wherein at least 70% of the limus active agent is in the form of microcrystals having a crystal size in the range of 10 μm to 50 μm.

The present invention preferably relates to a medical device selected from a catheter balloon, a balloon catheter, a stent, or a cannula, coated with at least one tri-O-acylglycerol selected from the group consisting of trioctanoylglycerol, trinononoylglycerol, tridecanoylglycerol, and triundecanoylglycerol, and at least one limus active agent in the form of microcrystals,
wherein the microcrystals of the at least one limus active agent have a crystal size in the range of 1 μm to 300 μm,
wherein the at least one limus active agent is selected from rapamycin (sirolimus) and everolimus,
wherein the at least one tri-O-acylglycerol and the at least one limus active agent are present in a mass ratio of 10%-30% tri-O-acylglycerol to 90%-70% limus active agent,
wherein at least 70% of the limus active agent is in the form of microcrystals having a crystal size in the range of 10 μm to 50 μm.

The present invention preferably relates to a medical device selected from a catheter balloon, a balloon catheter, a stent, or a cannula coated with at least one tri-O-acylglycerol selected from the group consisting of trioctanoylglycerol, trinononoylglycerol, tridecanoylglycerol, and triundecanoylglycerol and at least one limus active agent in the form of microcrystals,
wherein the at least one limus active agent has a crystallinity of at least 90% by weight,
wherein the at least one limus active agent is selected from rapamycin (sirolimus) and everolimus,
wherein the at least one tri-O-acylglycerol and the at least one limus active agent are present in a mass ratio of 10%-30% tri-O-acylglycerol to 90%-70% limus active agent,
wherein at least 70% of the limus active agent is in the form of microcrystals having a crystal size in the range of 10 μm to 50 μm.

For the preparation of the coatings, suspensions according to the invention are used which contain the limus active agent in the form of microcrystals together with at least one dissolved tri-O-acylglycerol selected from the group consisting of trioctanoylglycerol, trinonanoylglycerol, tridecanoylglycerol, and triundecanoylglycerol in a solvent or solvent mixture.

The present invention therefore relates to a medical device selected from a catheter balloon, a balloon catheter, a stent or a cannula coated with a suspension containing:
a) at least one tri-O-acylglycerol selected from the group consisting of trioctanoylglycerol, trinonanoylglycerol, tridecanoylglycerol, and triundecanoylglycerol;
b) at least one limus active agent in the form of microcrystals; and
c) a solvent or a solvent mixture wherein the at least one tri-O-acylglycerol dissolves and in which the microcrystals of the at least one limus active agent do not dissolve or do not dissolve when at least one tri-O-acylglycerol is present.

The present invention therefore relates to a medical device selected from a catheter balloon, a balloon catheter, a stent or a cannula, obtainable according to a method comprising the following steps:
a) providing a medical device selected from a catheter balloon, a balloon catheter, a stent, or a cannula, having a medical device surface;
b) providing a suspension comprising a tri-O-acylglycerol selected from the group consisting of trioctanoylglycerol, trinonanoylglycerol, tridecanoylglycerol, and triundecanoylglycerol dissolved in a solvent or a solvent mixture and at least one limus active agent in the form of microcrystals, wherein the microcrystals of the at least one limus active agent do not dissolve in the solvent or the solvent mixture or do not dissolve when the at least one tri-O-acylglycerol is present;
c) applying the suspension to the surface of the medical device by means of a syringe method, pipetting method, capillary method, fold spray method, dipping method, spraying method, dragging method, thread dragging method, drop dragging method, or rolling method,
d) drying the coating

In some embodiments of the present invention, a medical device having a medical device surface may be provided that has a base coating on the medical device surface. In such embodiments, the suspension according to the invention comprising at least one —O-acylglycerol selected from the group consisting of trioctanoylglycerol, trinonanoylglycerol, tridecanoylglycerol, and triundecanoylglycerol and at least one limus active agent in the form of microcrystals are applied on this base coating.

For example, the medical device surface may additionally be provided with a hemocompatible, athrombogenic layer as a base coating applied by covalent immobilization of semisynthetic heparin derivatives such as desulfated, reacetylated heparin or chitosan derivatives such as N-carboxymethylated, partially N-acetylated chitosan.

If necessary or advantageous, the implant surface can be pretreated e.g. by surface activation e.g. via plasma process, temperature treatment, wetting with suitable solvents, DLC coating (“diamond like carbon”), Teflon coating, or siliconization, etc. It could be shown that wetting with a suitable solvent has a positive effect on adhesion.

Likewise, a polymeric base coating with biodegradable and/or biostable polymers can be realized. These polymeric coatings can of course also contain additives, e.g. further active agents or mixtures of active agents, metals, salts etc. Suitable active agents or combinations of active agents include anti-inflammatory, cytostatic, cytotoxic, antiproliferative, anti-microtubule, antiangiogenic, antirestenotic (anti-restenosis), antifungal, antineoplastic, antimigrative, athrombogenic and antithrombogenic substances.

If the limus active agent in the form of microcrystals is not applied directly to or on the medical device surface, suitable biocompatible substances of synthetic, semisynthetic and/or native origin biostable and/or biodegradable polymers, or polysaccharides can be used as a carrier or matrix.

The following can be named as polymers that are generally biologically stable and only slowly biodegradable: polyacrylic acid and polyacrylates such as polymethyl methacrylate, polybutyl methacrylate, polyacrylamide, polyacrylonitrile, polyamides, polyetheramides, polyethyleneamine, polyimides, polycarbonates, polycarbourethanes, polyvinyl ketones, polyvinyl halides, polyvinylidene halides, polyvinyl ethers, polyvinyl aromatics, polyvinyl esters, polyvinyl pyrollidones, polyoxymethylenes, polyethylene, polypropylene, polytetrafluoroethylene, polyurethanes, polyolefin elastomers, Polyisobutylenes, EPDM rubbers, fluorosilicones, carboxymethyl chitosan, polyethylene terephthalate, polyvalerates, carboxymethyl cellulose, cellulose, rayon, rayon triacetate, cellulose nitrates, cellulose acetates, hydroxyethyl cellulose, cellulose butyrates, cellulose acetate butyrates, ethyl vinyl acetate copolymers, polysulfones, polyether sulfones, epoxy resins, ABS resins, EPDM rubbers, silicone prepolymers, silicones such as polysiloxanes, polyvinyl halogens and copolymers, cellulose ethers, cellulose triacetates, chitosan, chitosan derivatives, polymerizable oils such as linseed oil and copolymers. e.g. linseed oil, and copolymers and/or mixtures thereof.

As generally biodegradable, biodegradable or resorbable polymers can be used, for example: Polyvalerolactones, poly-ε-decalactones, polylactides, polyglycolides, copolymers of polylactides and polyglycolides, poly-ε-caprolactone, polyhydroxybutyric acid, polyhydroxybutyrates, polyhydroxyvalerates, polyhydroxybutyrate-co-valerates, poly(1,4-dioxane-2,3-diones), poly(1,3-dioxan-2-one), poly-para-dioxanones, polyanhydrides such as polymaleic anhydrides, polyhydroxymethacrylates, fibrin, polycyanoacrylates, polycaprolactone dimethylacrylates, poly-b-maleic acid, polycaprolactone butyl acrylates, multiblock polymers such as e.g. of oligocaprolactone diols and oligodioxanone diols, polyether ester multiblock polymers such as PEG and poly(butylene terephthalate, polypivotolactones, polyglycolic acid trimethyl carbonate, polycaprolactone glycolides, poly(g-ethylglutamate), poly(DTH-iminocarbonate), poly(DTE-co-DT-carbonate), Poly(bisphenol A-iminocarbonate), polyorthoester, polyglycol acid trimethyl carbonate, polytrimethyl carbonate, polyiminocarbonate, poly(N-vinyl) pyrolidone, polyvinyl alcohols, polyesteramides, glycolated polyesters, polyphosphoesters, polyphosphazenes, poly[p-carboxyphenoxy)propane], polyhydroxypentanoic acid, polyethylene oxide-propylene oxide, soft polyurethanes, polyurethanes with amino acid residues in the backbone, polyether esters such as polyethylene oxide, polyalkenoxalates, polyorthoesters and their copolymers, carrageenans, fibrinogen, starch, collagen, protein-based polymers, polyamino acids, synthetic polyamino acids, zein, modified zein, polyhydroxyalkanoates, pectic acid, actinic acid, modified and unmodified fibrin and casein, carboxymethyl sulfate, albumin, hyaluronic acid, heparan sulfate, heparin, chondroitin sulfate, dextran, b-cyclodextrins, copolymers with PEG and polypropylene glycol, gum arabic, guar, gelatin, collagen, collagen-N-hydroxysuccinimide, lipids and lipoids, polymerizable oils with a low degree of crosslinking, modifications and copolymers and/or mixtures of the above substances.

### DESCRIPTION OF THE FIGURES

FIG. 1 a) cross-sectional view of a circumferentially coated and partially folded balloon; b) microcrystalline structure of everolimus coating under SEM at 1000× magnification.

FIG. 2 shows at 200× magnification rapamycin in the form of microcrystals in rod shape with very narrow particle size distribution mainly in the range of 10 μm to 30 μm.

FIG. 3 shows at 1000× magnification rapamycin in the form of microcrystals in regular rod shape with very narrow particle size distribution mainly in the range of 10 μm to 30 μm.

FIG. 4 shows at 200× magnification rapamycin in the form of microcrystals in almost identical rod shape with extremely narrow particle size distribution mainly in the range of 15 μm to 30 μm. No larger crystals or agglomerates are visible.

FIG. 5 shows at 1000× magnification rapamycin in the form of microcrystals in almost perfectly regular rod shape with very narrow particle size distribution mainly in the range of 15 μm to 30 μm. The shape of rhombohedral prisms can be seen very clearly.

FIG. 6 shows at 200× magnification everolimus in the form of microcrystals in needle shape with extremely narrow particle size distribution mainly in the range of 20 μm to 40 μm. No larger crystals or agglomerates are visible.

FIG. 7 shows at 1000× magnification everolimus in the form of microcrystals in needle shape with extremely narrow particle size distribution mainly in the range of 20 μm to 40 μm. The needle shape can be seen very clearly.

FIG. 8 shows at 1000× magnification rapamycin in the form of microcrystals in almost identical rod shape with extremely narrow particle size distribution mainly in the range of 20 μm to 40 μm. No larger crystals or agglomerates are visible.

FIG. 9 shows at 1000× magnification rapamycin in the form of microcrystals in almost perfectly regular rod shape with very narrow particle size distribution mainly in the range of 20 μm to 40 μm. The shape of rhombohedral prisms can be seen very clearly.

FIG. 10 shows the microcrystalline structure of the rapamycin coating with microcrystals of rapamycin substantially in rhombohedral prisms under SEM at 1000× magnification.

FIG. 11 shows the microcrystalline structure of the rapamycin coating with milled microcrystals of rapamycin under SEM at 1000× magnification.

FIG. 12 shows the model formed from silicone tubing that mimics the natural course of vessels in the organism a) Simulated Peripheral Catheter; b) Simulated Femoral Artery.

FIG. 13 a) shows a bending test to determine the particle release of a coated catheter balloon; b) shows an edge impact test to determine the particle release of a coated catheter balloon.

FIG. 14 shows rapamycin coatings not according to the invention prepared according to WO 2015/039969 A1 at 1000× magnification. A too large almost round crystal surrounded by many quite small crystals of irregular shape and broad particle size distribution can be seen.

FIG. 15 shows rapamycin coatings not according to the invention prepared according to WO 2015/039969 A1 at 1200× magnification. Numerous quite large crystals surrounded by many quite small crystals of irregular shape and broad particle size distribution can be seen.

FIG. 16 a) shows rapamycin crystal coating with dry crystals not according to the invention; b) shows the coating from a) after “solvent bonding”. The crystals are no longer intact.

FIG. 17 a) shows a rapamycin crystal coating not according to the invention with dry crystals and base coating of adhesive and topcoat with trioctanoylglycerol. b) shows a rapamycin crystal coating not according to the invention with dry crystals and base coating and topcoat with trioctanoylglycerol. c) shows an enlargement of FIG. b. It can be clearly seen that the coating is not uniform and has areas, where no microcrystals are present.

### EXAMPLES

### Example 1

Preparation of Microcrystalline Rapamycin and Microcrystalline Everolimus

For the preparation of the crystal suspensions according to the present invention, microcrystals of rapamycin and everolimus were first provided. Crystallization processes for the preparation of crystalline sirolimus (rapamycin) and crystalline everolimus are known from the prior art. Crystallization processes well known from the prior art include:

Crystallization by cooling: The limus active agent can be dissolved in a solvent at room or higher temperature until saturation and brought to crystallization at lower temperature e.g. at 0° C. The crystal size distribution can be influenced by a controlled cooling rate. Both polar and non-polar organic solvents, such as toluene, acetontrile, ethyl formate, isopropyl acetate, isobutyl acetate, ethanol, dimethyl formamide, anisole, ethyl acetate, methyl ethyl ketone, methyl isopropyl ketone, tetrahydrofuran, nitromethane, proprionitrile are suitable solvents for crystallization of limus active agents.

Crystallization by addition of seed crystals: The limus active agent is dissolved to saturation in a solvent and crystallization is initiated by the addition of seed crystals to achieve a controlled reduction of supersaturation.

Crystallization by addition of anti-solvent: The active agent is dissolved in a solvent and then a non-solvent or water is added. Two-phase mixtures are also possible here. Polar organic solvents such as acetone, acetonitrile, ethyl acetate, methanol, ethanol, isopropyl alcohol, butanol, butyl methyl ether, tetrahydrofuran, dimethyl formamide or dimethyl sulfoxide can be used as solvents for dissolving the limus active agent. Suitable non-solvents include pentane, hexane, cyclohexane or heptane. The solvent mixture can be allowed to stand for crystallization, stirred or slowly concentrated or evaporated in vacuo. The crystal size and crystallinity of the drug can be influenced by controlled addition of the nonpolar solvent. Supersaturation should be slower to produce large crystals and faster to produce small crystals. Controlling the addition rate of the anti-solvent to control the crystal size is well known.

For the production of microcrystals, crystallization can also be assisted by ultrasound. It is generally known that crystal size can be influenced by means of ultrasound. In this context, ultrasound can be used at the beginning of crystallization to initiate crystallization and nucleation, with further crystal growth then proceeding unhindered so that larger crystals can grow. The application of continuous sonication of a supersaturated solution with ultrasound, on the other hand, leads to smaller crystals, as many nuclei are formed in this process, resulting in the growth of numerous small crystals. Another option is to sonicate with ultrasound in pulse mode to influence crystal growth in such a way that tailored crystal sizes are achieved.

Other pmethods known from the prior art, such as micronization, grinding or sieving, can also be used to provide the desired crystal sizes. One possibility is to grind the crystals, which can also be done during crystallization by wet grinding. Milling can be advantageous to obtain different crystal sizes, i.e. a broader crystal size distribution. Milling allows for any desired sizes in the crystal size range. More uniform crystal sizes can be provided by, for example, performing a special sieving process after isolation and drying. Special sieving devices known from the prior art can be used for this purpose. In the sieving process, the limus active agent crystals can be sieved through a stack of sieves, for example, and divided into different size ranges.

For the preparation of microcrystalline rapamycin and microcrystalline everolimus, crystallization procedures with controlled crystallizations were carried out. Rapamycin and everolimus could thus be obtained directly in the form of microcrystals, avoiding subsequent grinding or micronization. Crystallization was performed by addition of anti-solvents (ethyl acetate/heptane). After crystallization, the microcrystals of rapamycin or everolimus were isolated, washed (heptane) and dried. Optionally, further separation into different crystal sizes was then performed by sieving method to provide narrower crystal size distributions of the microcrystals.

To evaluate the crystal size, the crystal size distribution and the shape of the crystals, a sample was placed on the foil of a SEM sample plate. Representative images were taken at 200, 1000 and 3000× magnification for evaluation, with 200× magnification being suitable for good detection of so-called oversize grains (coarse particles). Size estimation is performed using the scaling of the SEM images.

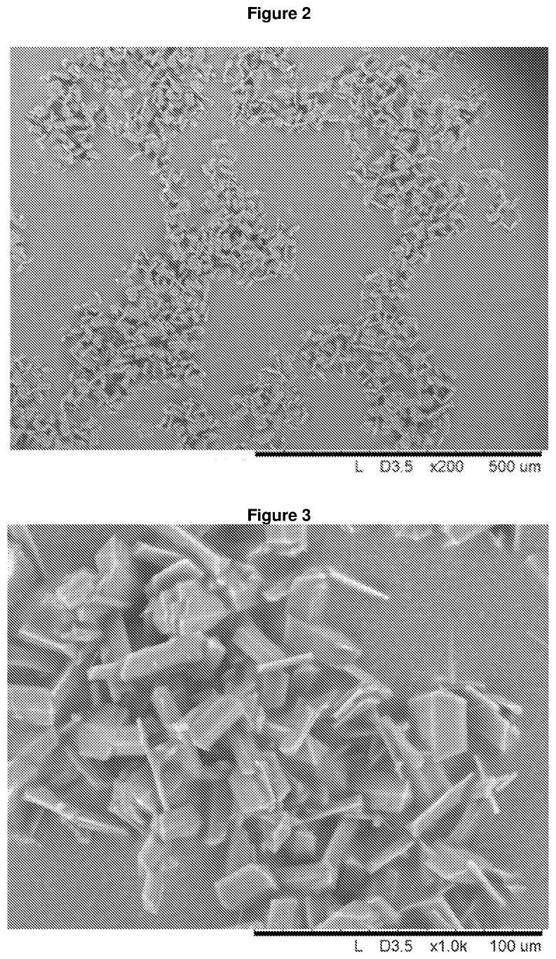

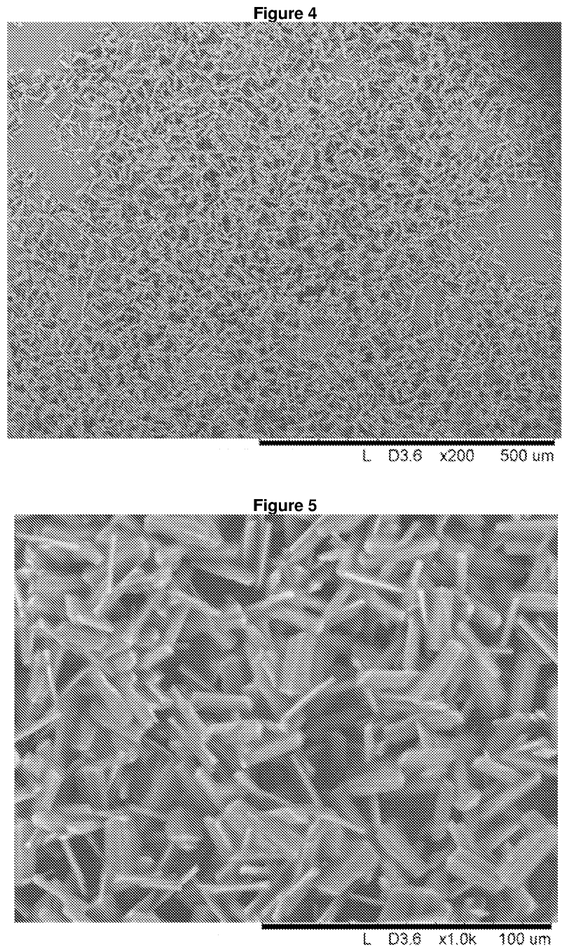

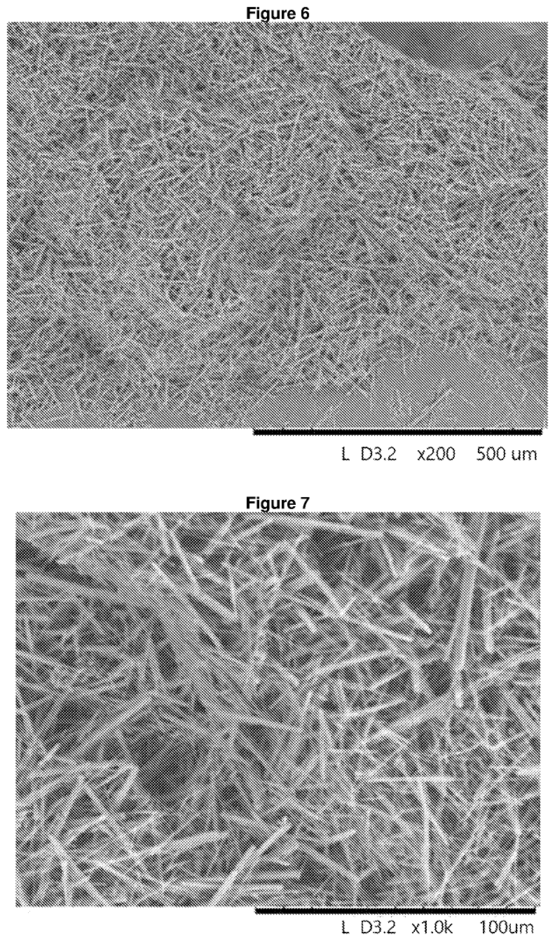

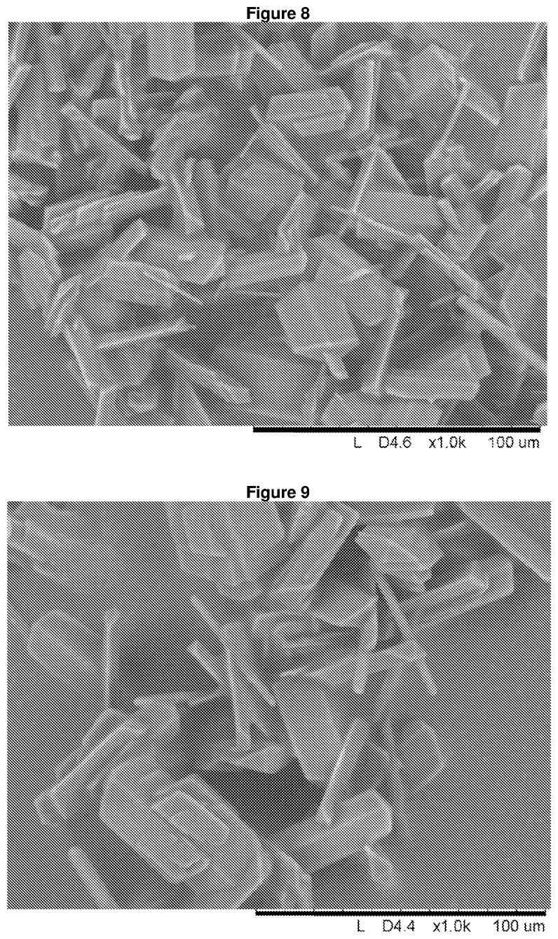

Example images of rapamycin in the form of microcrystals and everolimus in the form of microcrystals as used herein are shown in FIG. 2 to FIG. 9. In FIGS. 2 and 3, rapamycin in the form of microcrystals is shown in rod form with very narrow particle size distribution mainly in the range of 10 μm to 30 μm. In FIGS. 4 and 5, rapamycin is shown in the form of microcrystals in almost identical rod shape with extremely narrow particle size distribution mainly in the range of 15 μm to 30 μm. In FIGS. 7 and 8, rapamycin is shown in the form of microcrystals with a particle size distribution mainly in the range of 20 μm to 40 μm. In FIGS. 6 and 7, everolimus is shown in the form of microcrystals in needle shape with a particle size distribution mainly in the range of 20 μm to 40 μm. In all FIGS. 2 to 9, it can be seen that no larger crystals or agglomerates are present. It can also be clearly seen that everolimus is in the form of needles, while rapamycin is in the form of rhombohedral prisms.

The obtained microcrystals of rapamycin or everolimus were used to prepare crystal suspensions in the following examples.

### Example 2

Preparation of Crystal Suspensions with Tri-O-Acylglycerols and Microcrystalline Rapamycin (SIR) and Everolimus (EVR)

In the first step, solutions of tri-O-acylglycerols were first prepared in a solvent mixture. Subsequently, the solutions were combined with microcrystals of rapamycin and everolimus to investigate which tri-O-acylglycerols could be used to obtain stable crystal suspensions. The composition of the solvents and solvent mixture varied depending on the active agent used. The solutions and solvent mixtures prepared in the example apply to rapamycin (SIR) and everolimus (EVR). For the preparation of the solutions, an ethyl acetate/heptane solvent mixture was used here as an example.

1. Preparation of Solutions with Tri-O-Acylglycerols

To prepare the solutions, the respective tri-O-acylglycerol was first dissolved in a polar organic solvent and then a non-polar solvent was added. In addition, solutions with the addition of an antioxidant (BHT) were prepared as an alternative.

To prepare the solutions, 770 mg of the respective tri-O-acylglycerol and optionally 150 mg BHT were dissolved in 14 g ethyl acetate (15.6 mL). Then, 57.4 g of n-heptane (84.4 mL) was added. Subsequently, homogenization and filtration were performed. The total volume of the solvent mixture is 100 mL.

Solution 1a)

Tri-O-acylglycerol: Trioctanoylglycerol

Antioxidant: ---

Solution 1b)

Tri-O-acylglycerol: Trioctanoylglycerol

Antioxidant: BHT

Solution 1c)

Tri-O-acylglycerol: Tridecanoylglycerol

Antioxidant:

Solution 1d)

Tri-O-acylglycerol: Tridecanoylglycerol

Antioxidant: BHT

Solution 1e)

Tri-O-acylglycerol: Trihexanoylglycerol

Antioxidant:

Solution 1f)

Tri-O-acylglycerol: Trihexanoylglycerol

Antioxidant: BHT

Solution 1g)

Tri-O-acylglycerol: Tributanoylglycerol

Antioxidant:

Solution 1h)

Tri-O-acylglycerol: Tributanoylglycerol

Antioxidant: BHT

Solution 1i)

Tri-O-acylglycerol: 770 mg Triacetin

Antioxidant: ---

Solution 1i)

Tri-O-acylglycerol: Triacetin

Antioxidant: BHT

Solution 1k)

Tri-O-acylglycerol: Tridodecanoylglycerol

Antioxidant:

Solution 1l)

Tri-O-acylglycerol: Tridodecanoylglycerol

Antioxidant: BHT

Solution 1m)

Tri-O-acylglycerol: Citryl/Lactyl/Linoleyl/Oleyl-O-Glycerols (IMWITOR®)

Antioxidant:

Solution 1n)

Tri-O-acylglycerol: Citryl/Lactyl/Linoleyl/Oleyl-O-Glycerols (IMWITOR®)

Antioxidant: BHT

Solution 10)

Tri-O-acylglycerol: Dioctanoylglycerol

Antioxidant:

Solution 1p)

Tri-O-acylglycerol: Dioctanoylglycerol

Antioxidant: BHT

Solution 1q)

Tri-O-acylglycerol: Monooctanoylglycerol

Antioxidant: ---

Solution 1r)

Tri-O-acylglycerol: Monooctanoylglycerol

Antioxidant: BHT

Solution 1s)

Tri-O-acylglycerol: Tritetradecanoylglycerol

Antioxidant: ---

Solution 1t)

Tri-O-acylglycerol: Tritetradecanoylglycerol

Antioxidant: BHT

2. Redispersion of microcrystals of rapamycin and everolimus.

To a precisely weighed amount of dry microcrystals of the previously prepared limus active agent, a defined amount of the solutions containing tri-O-acylglycerol and optionally antioxidant is carefully added. It was investigated whether the microcrystals of the limus active agent are not soluble in the solutions and whether suspensions are formed.

To check whether crystal suspension can be prepared, to each 200 mg of rapamycin in the form of microcrystals or 200 mg of everolimus in the form of microcrystals were carefully added 10 mL of one of the solutions 1a) to 1t) at room temperature. Three mixtures of 10 mL of solution for each solution were prepared. After combining, it was tested whether the microcrystals of limus active agents dissolved directly in the solutions. For solutions where there was no immediate dissolution of the microcrystals of the limus active agents, the suspensions of the solutions without antioxidant were allowed to stand for a period of 100 h and tested again to see if the microcrystals of the limus active agents dissolved. For the suspensions of the solutions with antioxidant, the mixture was heated to 50° C. to check whether the suspensions remained stable under sterilization conditions.

TABLE 9
Overview of results for the preparation of crystal suspensions
with solutions containing different tri-O-acylglycerols (+++++ =
stable crystal suspension, uniform distribution of microcrystals,
intact microcrystals, “floating” of microcrystals; +++ =
suspension, no complete dissolution of microcrystals; +
= sedimentation or partial dissolution of microcrystals,
no intact crystals; −−− = no suspension; −− =
no suspension; − = no suspension; −/− =
not studied).

| | Active | | Suspension | Suspension |
|---|---|---|---|---|
| Solution | agent | | without BHT | with BHT |
| (10 mL) | (300 mg) | Suspension | (100 h) | (50° C.) |
| 1a) | Rapamycin | +++++ | +++++ | +++++ |
| 1b) | Rapamycin | +++++ | +++++ | +++++ |
| 1c) | Rapamycin | +++++ | +++++ | +++++ |
| 1d) | Rapamycin | +++++ | +++++ | +++++ |
| 1e) | Rapamycin | + | −−− | −−− |
| 1f) | Rapamycin | + | −−− | −−− |
| 1g) | Rapamycin | −−− | −/− | −/− |
| 1h) | Rapamycin | −−− | −/− | −/− |
| 1i) | Rapamycin | −− | −/− | −/− |
| 1j) | Rapamycin | −− | −/− | −/− |
| 1k) | Rapamycin | +++ | + | + |
| 1l) | Rapamycin | +++ | + | + |
| 1m) | Rapamycin | − | −/− | −/− |
| 1n) | Rapamycin | − | −/− | −/− |
| 1o) | Rapamycin | −− | −/− | −/− |
| 1p) | Rapamycin | −− | −/− | −/− |
| 1q) | Rapamycin | −− | −/− | −/− |
| 1r) | Rapamycin | −− | −/− | −/− |
| 1s) | Rapamycin | +++ | + | + |
| 1t) | Rapamycin | +++ | + | + |
| 1a) | Everolimus | +++++ | +++++ | +++++ |
| 1b) | Everolimus | +++++ | +++++ | +++++ |
| 1c) | Everolimus | +++++ | +++++ | +++++ |
| 1d) | Everolimus | +++++ | +++++ | +++++ |
| 1e) | Everolimus | + | −−− | −−− |
| 1f) | Everolimus | + | −−− | −−− |
| 1g) | Everolimus | −−− | −/− | −/− |
| 1h) | Everolimus | −−− | −/− | −/− |
| 1i) | Everolimus | −− | −/− | −/− |
| 1j) | Everolimus | −− | −/− | −/− |
| 1k) | Everolimus | +++ | + | + |
| 1l) | Everolimus | +++ | + | + |
| 1m) | Everolimus | − | −/− | −/− |
| 1n) | Everolimus | − | −/− | −/− |
| 1o) | Everolimus | −− | −/− | −/− |
| 1p) | Everolimus | −− | −/− | −/− |
| 1q) | Everolimus | −− | −/− | −/− |
| 1r) | Everolimus | −− | −/− | −/− |
| 1s) | Everolimus | +++ | + | + |
| 1t) | Everolimus | +++ | + | + |

Result:

Stable crystal suspensions could be prepared with the tri-O-acylglycerols trioctanoylglycerol and tridecanoylglycerol, which remained stable even after 100 h and under temperature elevation. The presence of the antioxidant did not affect the stability of the crystal suspension. A stable crystal suspension was obtained with the tri-O-acylglycerols trioctanoylglycerol and tridecanoylglycerol, with and without the presence of BHT. In the solutions with trioctanoylglycerol and tridecanoylglycerol, no sedimentation of the microcrystals occurred, the microcrystals “float” in the crystal suspension and are uniformly distributed.

To evaluate the crystal size, particle size distribution (i.e., crystal size distribution), and shape of the crystals, a sample was taken with a Pasteur pipette in each case and a drop was placed on the slide of the SEM sample plate. SEM images were taken at 200× and 1000× magnification for evaluation.

SEM images showed that the microcrystals of the crystal suspensions containing the tri-O-acylglycerols trioctanoylglycerol and tridecanoylglycerol remained intact. The microcrystals of everolimus were consistently in the form of needles, while the microcrystals of rapamycin continued to be in the form of rhombohedral prisms. The crystal size distribution also continued to correspond to the crystal size distribution of the microcrystalline everolimus or rapamycin originally used. Thus, no crystal growth or aggregation of microcrystals occurred in these crystal suspensions.

In the solutions with trihexanoylglycerol, the microcrystals of everolimus and rapamycin did not dissolve directly. In these suspensions, the microcrystals were not as uniformly present in contrast to the crystal suspensions with trioctanoylglycerol and tridecanoylglycerol. In the case of trihexanoylglycerol, the microcrystals of everolimus and rapamycin were almost completely dissolved after 100 h, and when the temperature was increased, the microcrystals of everolimus and rapamycin dissolved rapidly.

In the solutions with tridodecanoylglycerol and tritetradecanoylglycerol, the microcrystals of everolimus and rapamycin did not dissolve directly. However, these “suspensions” were also found to be unstable. In the case of tridodecanoylglycerol and tritetradecanoylglycerol, the microcrystals of everolimus and rapamycin did not dissolve completely after 100 h. However, the microcrystals of everolimus and rapamycin were still in a stable state. However, in contrast to the crystal suspensions with trioctanoylglycerol and tridecanoylglycerol, the microcrystals were not as uniformly distributed and sedimentation of the crystals occurred. An increase in temperature accelerated this process.

To evaluate the crystal size, particle size distribution (i.e., crystal size distribution), and shape of the crystals, one sample was taken from each using a Pasteur pipette and one drop was placed on the foil of the SEM sample plate. An additional sample was taken from the sediment and one drop was placed on the slide of the SEM sample plate. SEM images were taken at 200× and 1000× magnification for evaluation. SEM images were taken at 200 and 1000× magnification for evaluation.

SEM images showed that the microcrystals of the crystal suspensions containing tridodecanoylglycerol and tritetradecanoylglycerol did not remain intact. The crystal size distribution no longer matched the crystal size distribution of the microcrystalline everolimus or rapamycin originally used, and larger crystals were detected, especially in that of the sample taken from the sediment.

No crystal suspensions could be prepared with the other solutions of tri-O-acylglycerols. Thus, stable crystal suspensions could only be prepared with trioctanoylglycerol and tridecanoylglycerol.

### Example 3

Preparation of Crystal Suspensions with Additional Tri-O-Acylglycerols and Microcrystalline Rapamycin (SIR) and Everolimus (EVR)

Based on the results from Example 2, further solutions of the three tri-O-acylglycerols triheptanoylglycerol, trinonanoylglycerol and triundecanoylglycerol were prepared to investigate whether stable crystal suspensions can be obtained with these. An ethyl acetate/heptane solvent mixture was also used here to prepare the solutions.

1. Preparation of Solutions with Tri-O-Acylqlycerols

To prepare the solutions, 770 mg of the respective tri-O-acylglycerol and optionally 150 mg BHT were dissolved in 14 g ethyl acetate (15.6 mL). Then, 57.4 g of n-heptane (84.4 mL) was added. Subsequently, homogenization and filtration were performed. The total volume of the solvent mixture is 100 mL.

Solution 2a)

Tri-O-acylglycerol: Triheptanoylglycerol

Antioxidant: ---

Solution 2b)

Tri-O-acylglycerol: Triheptanoylglycerol

Antioxidant: BHT

Solution 2c)

Tri-O-acylglycerol: Trinonanoylglycerol

Antioxidant: ---

Solution 2d)

Tri-O-acylglycerol: Trinonanoylglycerol

Antioxidant: BHT

Solution 2e)

Tri-O-acylglycerol: Triundecanoylglycerol

Antioxidant: ---

Solution 2f)

Tri-O-acylglycerol: Triundecanoylglycerol

Antioxidant: BHT

2. Redispersion of microcrystals of rapamycin and everolimus.

To prepare crystal suspension, 200 mg of rapamycin in the form of microcrystals or 200 mg of everolimus in the form of microcrystals each were carefully added to 10 mL of of solutions 2a) to 2f) at room temperature, as in Example 2. Three mixtures of 10 mL of solution for each solution were prepared. After combining, it was tested whether the microcrystals of limus active agents dissolved directly in these solutions. For solutions where there was no immediate dissolution of the microcrystals of the limus active agents, the suspensions of the solutions without antioxidant were allowed to stand for a period of 100 hours and tested again to see if the microcrystals of the limus active agents dissolved. For the suspensions of the solutions with antioxidant, the mixture was heated to 50° C. to check whether the suspensions remained stable under sterilization conditions.

For direct comparison, the results from example 2 for solutions 1a) to 1d) are included in the following table.

TABLE 10
Overview results for the preparation of crystal suspensions with solutions
containing different tri-O-acylglycerols (+++++ =
excellent stable crystal suspension, uniform distribution of microcrystals,
intact microcrystals, “floating” of microcrystals; ++++ =
stable crystal suspension, uniform distribution of microcrystals,
intact microcrystals, “floating” of microcrystals; +++ =
crystal suspension,; ++ = suspension, no complete dissolution
of microcrystals; + = sedimentation or partial dissolution
of microcrystals, no intact crystals).

| | Active | | Suspension | Suspension |
|---|---|---|---|---|
| Solution | agent | | without BHT | with BHT |
| (10 mL) | (300 mg) | Suspension | (100 h) | (50° C.) |
| 1a) | Rapamycin | +++++ | +++++ | +++++ |
| 1b) | Rapamycin | +++++ | +++++ | +++++ |
| 1c) | Rapamycin | +++++ | +++++ | +++++ |
| 1d) | Rapamycin | +++++ | +++++ | +++++ |
| 2a) | Rapamycin | +++ | + | − |
| 2b) | Rapamycin | +++ | + | − |
| 2c) | Rapamycin | +++++ | ++++ | ++++ |
| 2d) | Rapamycin | +++++ | ++++ | ++++ |
| 2e) | Rapamycin | +++++ | ++++ | ++++ |
| 2f) | Rapamycin | +++++ | ++++ | ++++ |
| 1a) | Everolimus | +++++ | +++++ | +++++ |
| 1b) | Everolimus | +++++ | +++++ | +++++ |
| 1c) | Everolimus | +++++ | +++++ | +++++ |
| 1d) | Everolimus | +++++ | +++++ | +++++ |
| 2a) | Everolimus | +++ | + | − |
| 2b) | Everolimus | +++ | + | − |
| 2c) | Everolimus | +++++ | ++++ | ++++ |
| 2d) | Everolimus | +++++ | ++++ | ++++ |
| 2e) | Everolimus | +++++ | ++++ | ++++ |
| 2f) | Everolimus | +++++ | ++++ | ++++ |

Results:

The tri-O-acylglycerols trinonanoylglycerol and triundecanoylglycerol were used to prepare stable crystal suspensions that remained stable after 24-48 hours and under temperature elevation. The presence of the antioxidant had no effect on the stability of the crystal suspension. In the solutions with trinonanoylglycerol and triundecanoylglycerol, no sedimentation of the microcrystals occurred; the microcrystals “floated” in the crystal suspension and were uniformly distributed.

To evaluate the crystal size, particle size distribution (i.e., crystal size distribution), and shape of the crystals, a sample was taken with a Pasteur pipette in each case and a drop was placed on the slide of the SEM sample plate. SEM images were taken at 200× and 1000× magnification for evaluation.

SEM images showed that the microcrystals of the crystal suspensions containing the tri-O-acylglycerols trinonanoylglycerol and triundecanoylglycerol remained intact. The microcrystals of everolimus were consistently in the form of needles, while the microcrystals of rapamycin continued to be in the form of rhombohedral prisms. The crystal size distribution also continued to correspond to the crystal size distribution of the microcrystalline everolimus or rapamycin originally used.

In the solutions with triheptanoylglycerol, the microcrystals of everolimus and rapamycin did not dissolve directly. However, the microcrystals of everolimus and rapamycin were partially dissolved after 24-48h, and under increasing the temperature, the microcrystals of everolimus and rapamycin dissolved.

To evaluate the crystal size, particle size distribution (i.e., crystal size distribution), and shape of the crystals, a sample was taken with a Pasteur pipette in each case and a drop was placed on the slide of the SEM sample plate. SEM images were taken at 200× and 1000× magnification for evaluation.

SEM images showed that the microcrystals of the crystal suspensions with the triheptanoylglycerol did not remain intact. The crystal size distribution no longer matched the crystal size distribution of the microcrystalline everolimus or rapamycin originally used.

Stable crystal suspensions could thus be prepared with the further tri-O-acylglycerols trinonanoylglycerol and triundecanoylglycerol.

### Example 4

Preparation of 3% and 1% Crystal Suspensions Containing Trioctanoylglycerol and Microcrystalline Everolimus (EVR)

I. Preparation of the Solutions of Trioctanoylglycerol

Ia)

Solvent mixture example for a 100 ml batch with 3% EVR crystal content. In 14 g ethyl acetate, 770 mg trioctanoylglycerol is dissolved. To this solution, 57.4 g of n-heptane is added, homogenized and filtered.

Ib) Solvent mixture example for a 100 ml batch with 3% EVR crystal content and BHT. In 14 g ethyl acetate, 770 mg trioctanoylglycerol and 150 mg BHT are dissolved. To this solution, 57.4 g of n-heptane is added, homogenized and filtered.

Ic) Solution mixture example for a 100 ml batch with 1% EVR crystal content. In 14 g ethyl acetate 250 mg trioctanoylglycerol, and 20 mg Tween 80 are dissolved. To this solution 57.4 g of n-heptane is added, homogenized and filtered.

Id) Solution mixture example for a batch of 100 ml 1% EVR crystal content. In 14 g ethyl acetate, 250 mg trioctanoylglycerol, 50 mg BHT and 20 mg Tween 80 are dissolved. To this solution 57.4 g of n-heptane is added, homogenized and filtered.

II. Preparation of the Crystal Suspension

A defined quantity of the solvent mixture is carefully added to a precisely weighed quantity of dry active agent crystals prepared in advance. The crystals, which are insoluble in the solvent mixture, form a suspension with the solvent mixture. For solutions Ia) and Ib), 3 g of everolimus in the form of microcrystals were used and for solutions Ic) and Id), 1 g of everolimus in the form of microcrystals were used.

### Example 5

Preparation of crystal suspensions containing microcrystals of everolimus (EVR) and rapamycin with different proportions of tri-O-acylglycerols

In Examples 2 and 3, it was shown that for the tri-O-acylglycerols trioctanoylglycerol, tridecanoylglycerol, trinonanoylglycerol, and triundecanoyl-glycerol, stable crystal suspensions could be obtained at a mass ratio of tri-O-acylglycerols to microcrystals of limus active agent of 20:80.

For this purpose, further investigation was carried out using solutions of trioctanoylglycerol or tridecanoylglycerol with different proportions of tri-O-acylglycerol to find out the optimum mass ratio of tri-O-acylglycerol to microcrystalline limus active agent. To prepare the solutions, the appropriate amount of each tri-O-acylglycerol was dissolved in 14 g ethyl acetate (15.6 mL). Then, 57.4 g of n-heptane (84.4 mL) was added. Subsequently, homogenization and filtration were performed. The total volume of the solvent mixture is 100 mL.

Solution 3a)

Tri-O-acylglycerol: Trioctanoylglycerol

Weighing-in: 300 mg

Solution 3b)

Tri-O-acylglycerol: Trioctanoylglycerol

Weighing-in: 450 mg

Solution 3c)

Tri-O-acylglycerol: Trioctanoylglycerol

Weighing-in: 600 mg

Solution 3d)

Tri-O-acylglycerol: Trioctanoylglycerol

Weighing-in: 900 mg

Solution 3e)

Tri-O-acylglycerol: Trioctanoylglycerol

Weighing-in: 1200 mg

Solution 3f)

Tri-O-acylglycerol: Trioctanoylglycerol

Weighing-in: 1500 mg

Solution 4a)

Tri-O-acylglycerol: Tridecanoylglycerol

Weighing-in: 300 mg

Solution 4b)

Tri-O-acylglycerol: Tridecanoylglycerol

Weighing-in: 450 mg

Solution 4c)

Tri-O-acylglycerol: Tridecanoylglycerol

Weighing-in: 600 mg

Solution 4d)

Tri-O-acylglycerol: Tridecanoylglycerol

Weighing-in: 900 mg

Solution 4e)

Tri-O-acylglycerol: Tridecanoylglycerol

Weighing-in: 1200 mg

Solution 4f)

Tri-O-acylglycerol: Tridecanoylglycerol

Weighing-in: 1500 mg

TABLE 11
Overview results for the preparation of crystal suspensions with solutions
containing different amounts of tri-O-acylglycerol (+++++ =
excellent stable crystal suspension, uniform distribution of microcrystals,
intact microcrystals, “floating” of microcrystals; ++++ =
stable crystal suspension, uniform distribution of microcrystals,
intact microcrystals, “floating” of microcrystals; +++ =
crystal suspension; ++ = less stable crystal suspension;
+ = less stable crystal suspension, too viscous).

| | Active | |
|---|---|---|
| Solution | substance | Suspension |
| (10 mL) | (300 mg) | (100 h) |
| 1a) | Rapamycin | +++++ |
| 3a) | Rapamycin | +++ |
| 3b) | Rapamycin | ++++ |
| 3c) | Rapamycin | +++++ |
| 3d) | Rapamycin | ++++ |
| 3e) | Rapamycin | +++ |
| 3f) | Rapamycin | ++ |
| 1c) | Rapamycin | +++++ |
| 4a) | Rapamycin | +++ |
| 4b) | Rapamycin | ++++ |
| 4c) | Rapamycin | +++++ |
| 4d) | Rapamycin | ++++ |
| 4e) | Rapamycin | +++ |
| 4f) | Rapamycin | ++ |
| 1a) | Everolimus | +++++ |
| 3a) | Everolimus | +++ |
| 3b) | Everolimus | ++++ |
| 3c) | Everolimus | +++++ |
| 3d) | Everolimus | ++++ |
| 3e) | Everolimus | +++ |
| 3f) | Everolimus | ++ |
| 1c) | Everolimus | +++++ |
| 4a) | Everolimus | +++ |
| 4b) | Everolimus | ++++ |
| 4c) | Everolimus | +++++ |
| 4d) | Everolimus | ++++ |
| 4e) | Everolimus | +++ |
| 4f) | Everolimus | ++ |

Results:

With different proportions of the tri-O-acylglycerols trioctanoylglycerol and tridecanoylglycerol, stable crystal suspensions could be prepared with microcrystalline everolimus and microcrystalline rapamycin. For the solutions with different proportions of trioctanoylglycerol and tridecanoylglycerol, in particular, the crystal suspension with a ratio of 20:80 was still excellent stable after 100 h.

### Example 6

Preparation of Crystal Suspensions Containing Microcrystalline Rapamycin (SIR) and Everolimus (EVR) in Different Solvent Mixtures

Based on the results of the previous examples, different solvent mixtures for the preparation of crystal suspensions of microcrystalline rapamycin and everolimus were tested. The solvent mixture of ethyl acetate/heptane used in the previous examples has a ratio of about 85:15 (heptane:ethyl acetate).

To investigate further solvent mixtures for the preparation of the crystal suspensions, solvent mixtures of the polar organic solvents acetone, ethanol, iso-propanol and ethyl acetate and the non-polar organic solvents hexane, heptane and cyclohexane were prepared in different proportions.

To prepare the solutions, 770 mg of trioctanoylglycerol was dissolved in the polar solvent. Then the nonpolar solvent was added. It was then homogenized and filtered. The total volume of the solvent mixture is 100 mL in each case.

To test whether crystal suspensions could be prepared, 10 mL of each of the solutions was carefully added to 200 mg of everolimus in the form of microcrystals at room temperature. After combining, it was tested whether stable crystal suspensions were obtained.

TABLE 12
Overview of results for the preparation of crystal suspensions
various solvent mixtures (+++ = good; +/− = average; −−− = poor)

| | Non-polar | Ratio polar/ | |
|---|---|---|---|
| Polar organic | organic | non-polar | Crystal |
| solvent | solvent | solvent (v/v) | suspension |
| Ethyl acetate | Heptane | 10:90 | +++ |
| Ethyl acetate | Heptane | 30:70 | +++ |
| Ethyl acetate | Heptane | 40:60 | +++ |
| Ethyl acetate | Heptane | 50:50 | +/− |
| Ethyl acetate | Heptane | 60:40 | +/− |
| Ethyl acetate | Hexane | 10:90 | +++ |
| Ethyl acetate | Hexane | 30:70 | +++ |
| Ethyl acetate | Hexane | 40:60 | +++ |
| Ethyl acetate | Hexane | 50:50 | +/− |
| Ethyl acetate | Hexane | 60:40 | +/− |
| Ethyl acetate | Cyclohexane | 10:90 | +++ |
| Ethyl acetate | Cyclohexane | 30:70 | +++ |
| Ethyl acetate | Cyclohexane | 40:60 | +++ |
| Ethyl acetate | Cyclohexane | 50:50 | +/− |
| Ethyl acetate | Cyclohexane | 60:40 | +/− |
| Ethanol | Heptane | 10:90 | +++ |
| Ethanol | Heptane | 30:70 | ++ |
| Ethanol | Heptane | 40:60 | +/− |
| Ethanol | Heptane | 50:50 | −−− |
| Ethanol | Heptane | 60:40 | −−− |
| Ethanol | Hexane | 10:90 | +++ |
| Ethanol | Hexane | 30:70 | ++ |
| Ethanol | Hexane | 40:60 | +/− |
| Ethanol | Hexane | 50:50 | −−− |
| Ethanol | Hexane | 60:40 | −−− |
| Ethanol | Cyclohexane | 10:90 | +++ |
| Ethanol | Cyclohexane | 30:70 | ++ |
| Ethanol | Cyclohexane | 40:60 | +/− |
| Ethanol | Cyclohexane | 50:50 | −−− |
| Ethanol | Cyclohexane | 60:40 | −−− |
| Acetone | Heptane | 10:90 | +++ |
| Acetone | Heptane | 30:70 | +/− |
| Acetone | Heptane | 40:60 | +/− |
| Acetone | Heptane | 50:50 | −−− |
| Acetone | Heptane | 60:40 | −−− |
| Acetone | Hexane | 10:90 | +++ |
| Acetone | Hexane | 30:70 | +/− |
| Acetone | Hexane | 40:60 | +/− |
| Acetone | Hexane | 50:50 | −−− |
| Acetone | Hexane | 60:40 | −−− |
| Acetone | Cyclohexane | 10:90 | +++ |
| Acetone | Cyclohexane | 30:70 | +/− |
| Acetone | Cyclohexane | 40:60 | +/− |
| Acetone | Cyclohexane | 50:50 | −−− |
| Acetone | Cyclohexane | 60:40 | −−− |
| iso-propanol | Heptane | 10:90 | +++ |
| iso-propanol | Heptane | 30:70 | +/− |
| iso-propanol | Heptane | 40:60 | +/− |
| iso-propanol | Heptane | 50:50 | −−− |
| iso-propanol | Heptane | 60:40 | −−− |
| iso-propanol | Hexane | 10:90 | +++ |
| iso-propanol | Hexane | 30:70 | +/− |
| iso-propanol | Hexane | 40:60 | +/− |
| iso-propanol | Hexane | 50:50 | −−− |
| iso-propanol | Hexane | 60:40 | −−− |
| iso-propanol | Cyclohexane | 10:90 | +++ |
| iso-propanol | Cyclohexane | 30:70 | +/− |
| iso-propanol | Cyclohexane | 40:60 | +/− |
| iso-propanol | Cyclohexane | 50:50 | −−− |
| iso-propanol | Cyclohexane | 60:40 | −−− |

Stable crystal suspensions could be prepared with microcrystalline everolimus using various solvent mixtures. It has been shown that a content of at least 50% by volume of nonpolar solvent leads to very stable crystal suspensions.

### Example 7

Coatings of Balloon Catheters with Crystal Suspensions of Microcrystalline Everolimus

Balloon catheters 4×40 mm were coated with a 2% EVR suspension containing trioctanoylglycerol (20 wt % based on EVR) using a droplet dosing technique in a microdosing method such as the pipetting method, or droplet dragging method. It was possible to produce a uniform coating throughout with an equally uniform concentration of active agent on the balloon surface, where the crystals are evenly distributed. The studies on the recovery rate of active agent on balloon catheters divided into equally sized segments confirm the uniformity of the coating and thus the success in using a crystal suspension as well as a 100% recovery rate (see table 13).

TABLE 13
Balloon catheter 4 × 40 mm, coated with a 2% EVR
suspension (3 cuts as equal as possible into 4 segments).

| | | | Total |
|---|---|---|---|
| Samples | EVR | | [%/total] |
| catheter | content | Recovery | Target |
| segments | [μg/segm] | [%/segm] | 100% |
| Balloon 1: | | | |
| Distal 1 | 359.1 | 95.3 | 108.9 |
| Mid 1 | 402.6 | 106.8 | |
| Mid 1 | 436.8 | 115.9 | |
| Proximal 1 | 429.9 | 114.0 | |
| Balloon 2: | | | |
| Distal 2 | 357.9 | 94.9 | 111.3 |
| Mid 2 | 453.3 | 120.2 | |
| Mid 2 | 511.3 | 135.6 | |
| Proximal 2 | 353.7 | 93.8 | |
| Balloon 3: | | | |
| Distal 3 | 380.5 | 100.9 | 109.4 |
| Mid 3 | 421.9 | 111.9 | |
| Mid 3 | 452.2 | 119.9 | |
| Proximal 3 | 389.3 | 103.3 | |

Balloon catheters 7×150 mm were coated with a 2% EVR suspension containing trioctanoylglycerol (20 wt % based on EVR) using a droplet dosing technique in a microdosing process such as the pipetting process or droplet dragging process. It was possible to produce a uniform coating throughout with an equally uniform concentration of active agent on the balloon surface, ensuring that the crystals were evenly distributed. The studies on the recovery rate of active agent on balloon catheters divided into segments of equal size confirm the uniformity of the coating and thus the success in using a crystal suspension as well as a 100% recovery rate (see table 14).

TABLE 14
Balloon catheter 7 × 150 mm, coated with a 2% EVR suspension
(14 sections as equally as possible in 15 segments).

| | | | Total |
|---|---|---|---|
| Samples | EVR | | [%/total] |
| catheter | content | Recovery | Target |
| segments | [μg/segm] | [%/segm] | 100% |
| Distal 1 | 731.6 | 110.9 | 102.3% |
| mid 2 | 790.2 | 119.8 | |
| mid 3 | 792.4 | 120.1 | |
| mid 4 | 1027.6 | 155.8 | |
| mid 5 | 732.9 | 111.1 | |
| mid 6 | 625.1 | 94.8 | |
| mid 7 | 572.2 | 86.7 | |
| mid 8 | 682.4 | 103.4 | |
| mid 9 | 527.7 | 80.0 | |
| mid 10 | 562.0 | 85.2 | |
| mid 11 | 594.5 | 90.1 | |
| mid 12 | 780.1 | 118.2 | |
| mid 13 | 759.8 | 115.2 | |
| mid14 | 650.7 | 98.6 | |
| Proximal 15 | 194.8 | 44.7 | |

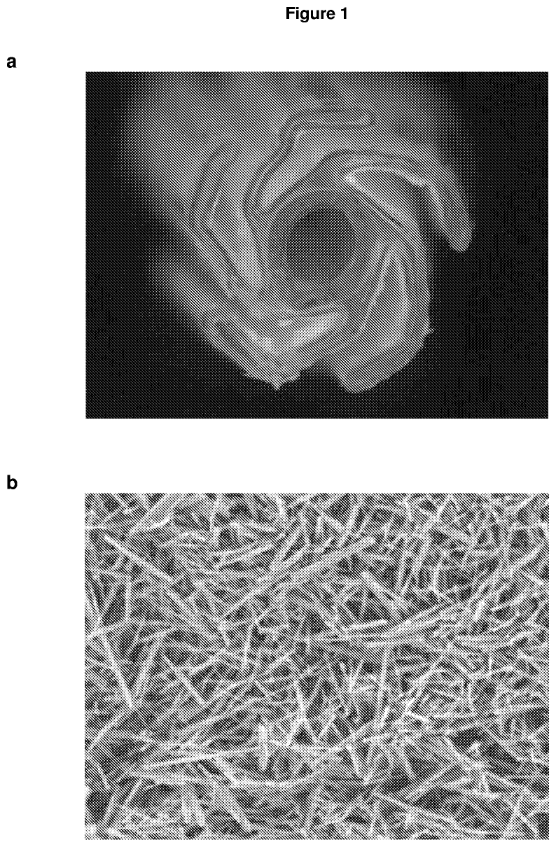

SEM images of the coatings. FIG. 1 a) shows a cross-sectional view of a circumferentially coated and partially folded balloon and b) shows the microcrystalline structure of the everolimus coating under SEM at 1000× magnification.

### Example 8

Coatings of Balloon Catheters with Crystal Suspensions of Microcrystalline Rapamycin (SIR)

Crystal Suspension of Rapamycin (SIR) Containing Trioctanoylqlycerol

Two different 2% crystal suspensions of microcrystalline rapamycin (SIR) containing trioctanoylglycerol (20 wt % based on EVR) were provided for balloon catheter coatings.

The first crystal suspension was prepared with rapamycin in the form of microcrystals with a particle size distribution in the range of 20 μm to 40 μm. Rapamycin is substantially completely present here in the form of rhombohedral prisms.

The second crystal suspension was prepared with rapamycin in the form of microcrystals, and the crystals of rapamycin were previously milled to provide a broader crystal size distribution.

Balloon catheters 4×40 mm were each coated with a 2% SIR suspension containing trioctanoylglycerol (20 wt % based on EVR) using a droplet dosing technique in a microdosing process such as the pipetting process or droplet dragging process. It was possible to produce a consistently uniform coating with likewise uniform drug concentration on the balloon surface. The studies on the recovery rate of active agent on balloon catheters divided into segments of equal size confirm the uniformity of the coating and thus the success in using a crystal suspension as well as a 100% recovery rate.

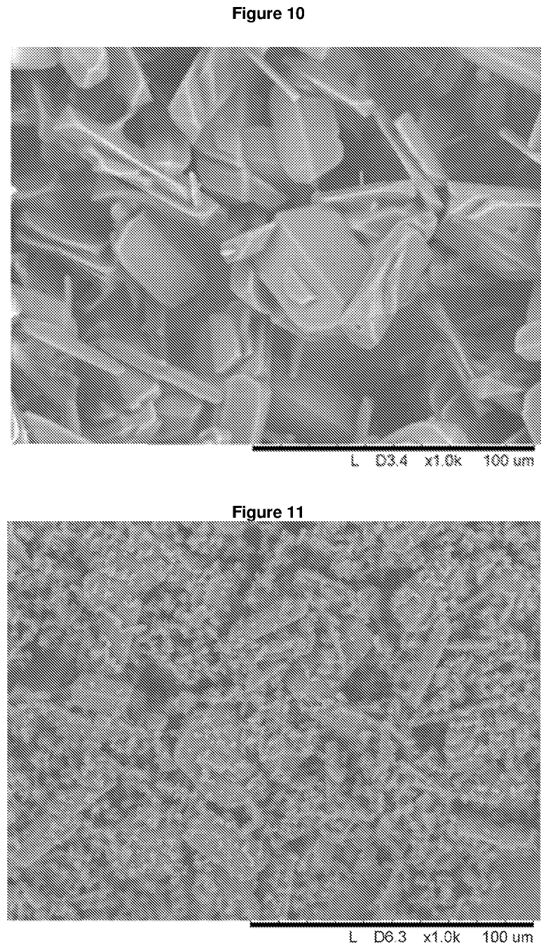

SEM images of the coatings. In FIG. 10, the microcrystalline structure of the rapamycin coating is shown to be substantially completely in the form of rhombohedral prisms of microcrystals of rapamycin (without milling) under SEM at 1000× magnification.

FIG. 11 shows the microcrystalline structure of the rapamycin coating with milled microcrystals of rapamycin under SEM at 1000× magnification.

Subsequently, these coated balloon catheters were struck against an edge of a suitable object over a black pad (edge impact test). The particles collected on the pad were then determined microscopically and the size distribution of the detached coating was determined. The inflated balloon was then immersed in PBS solution to allow any remaining particles still loosely adhering to also fall off and be included in the evaluation. In a further investigation, additional coated balloon catheters were inflated as prescribed over a black pad and bent in different directions (bending test). Particles collected on the pad were then determined microscopically and the size distribution of the detached coating was determined. The inflated balloon was then immersed in PBS solution so that any remaining loosely adhering particles could also fall off and be included in the evaluation.

Crystal Suspension of Rapamycin (SIR) Containing Triacylglycerols not According to the Invention

In Example 2, the microcrystalline active agent was found not to dissolve completely in the solutions containing tridodecanoylglycerol or tritetradecanoylglycerol. The suspensions containing tridodecanoylglycerol or tritetradecanoylglycerol were found to be unstable. To investigate the stability, flexibility and adhesion of coatings of balloon catheters with of microcrystalline rapamycin, suspensions of microcrystalline rapamycin (SIR) containing tridodecanoylglycerol or tritetradecanoylglycerol (20 wt % based on EVR) not according to the invention were freshly prepared and used directly for coating. Rapamycin was provided for this purpose in the form of microcrystals with a particle size distribution in the range of 20 μm to 40 μm.

Balloon catheters 4×40 mm were each coated with a 2% SIR suspension containing tridodecanoylglycerol or tritetradecanoylglycerol (20 wt % based on EVR) using a drop dosing technique in a micro dosing method such as the pipetting method or drop dragging method. It was found that the coating was not applied sufficiently uniformly with these suspensions.

Subsequently, these coated balloon catheters were struck against an edge of a suitable object over a black pad (edge impact test). The particles collected on the pad were then determined microscopically and the size distribution of the detached coating was determined. The inflated balloon was then immersed in PBS solution to allow any remaining particles still loosely adhering to also fall off and be included in the evaluation. In a further investigation, additional coated balloon catheters were inflated as prescribed over a black pad and bent in different directions (bending test). Particles collected on the pad were then determined microscopically and the size distribution of the detached coating was determined. The inflated balloon was then immersed in PBS solution so that any remaining loosely adhering particles could also fall off and be included in the evaluation.

Comparison of the Results of the Edge Impact Test and Bending Test of the Coatings

According to the Invention and the Coatings not According to the Invention

The edge impact test and the bending test clearly showed that the total particle loss as well as the particle size distribution/balloon surface area for the coating with trioctanoylglycerol according to the invention is far below that of the coatings with tridodecanoylglycerol or tritetradecanoylglycerol not according to the invention. The particle release for the coating according to the invention with microcrystals of rapamycin and trioctanoylglycerol shows that almost no particles are detached. The balloon catheters coated according to the invention have a determined particle count far below the prior art.

### Example 9

Particle Release (“Crumble Test”), Determination of the Loss of Active Agent or Coating During Implantation Using an In Vitro Model, Pre-Wetting of Implant Surfaces, Determination of a Uniform Coating

1. Particle Release (“Crumble Test”)

As a check on the mechanical adhesion of a coating on a surface, particle release is measured (“crumble test”), wherein it is determined, how many particles and of what size are released from the surface and thus lost when the coated medical device is impacted on edges and are bent (during and after inflation of the balloon). For this purpose, the coated implants are subjected to up to three mechanical tests. The weighted coated implant is weighed before and after testing.

a) Edge Impact Test

The coated balloon catheter is lightly struck against a hard (sharp) edge of a suitable object over a black pad. Particles collected on the pad are then determined microscopically and the size distribution of the detached coating is determined. The inflated balloon is then immersed in PBS solution. This causes any remaining loosely adhering particles to also fall off and can be included in the evaluation.

b) Bending Test

The coated balloon catheter is inflated as prescribed and bent by hand in various directions over a black pad. Particles collected on the pad are then determined microscopically and the size distribution of the detached coating is determined. The inflated balloon is then immersed in PBS solution. This causes any remaining loosely adhering particles to also fall off and can be included in the evaluation.

c) Adhesion Test

For this purpose, especially in the case of longer balloon catheters, e.g. peripheral balloons with a length of 150 mm are deflated as well as inflated, wrapped around a round vessel (e.g. test tube, standing cylinder or similar) with a suitable diameter and checked whether, on the one hand, no crumbs are formed and, on the other hand, it is checked whether the coating detaches from the balloon catheter and adheres to the surface of the vessel or not. Bend around a smooth object (preferably a glass laboratory vessel that fits from the circumference so that the catheter can be bent sufficiently and check if and if how much falls on the black pad. The hydrolysis tubes have a diameter of 12.8 mm. The balloon is bent around the hydrolysis tube in such a way that it is in contact with the wall. Smeary abrasion on the glass is tolerable, crumbling mass is not tolerable.

The “crumble tests” clearly show that the total particle loss as well as the particle size distribution/balloon surface area of all samples is far below the FDA guidelines. With trioctanoylglycerol, it can be clearly seen in Table 15 that regardless of loading with 1 μg/mm² EVR microcrystals or 3 μg/mm² EVR microcrystals, it releases significantly fewer particles and is well below the values obtained for commercially available and thus approved coated balloon catheters. Particle release in all measured particle size ranges for the coating with microcrystals of everolimus and trioctanoylglycerol according to the invention (here on different BMT catheter balloons) shows that virtually no particles can be detached. All balloon catheters coated according to the invention have a determined particle count far below the state of the art.

Thus, it can be seen that the flexibility and stability of the crystal coatings of microcrystals of everolimus-coated balloons according to the invention are far above the approved standard and state of the art.

TABLE 15
Number of particle losses/mm² balloon surface as a function
of particle size with different drug loading with 1 μg EVR/mm2
and 3 μg EVR/mm² with trioctanoylglycerol (*HTQ = Hemoteq,
trioctanoylglycerol 20 wt. % with respect to EVR)

| Released | HTQ* | HTQ | HTQ | HTQ | HTQ |
|---|---|---|---|---|---|
| Particles/mm² | Trioctanoyl- | Trioctanoyl- | Trioctanoyl- | Trioctanoyl- | Trioctanoyl- |
| Balloon surface | glycerol | glycerol | glycerol | glycerol | glycerol |
| Balloon size | 6.0 × 40 | 6.0 × 40 | 6.0 × 40 | 5.0 × 40 | 2.0 × 40 |
| [mm] | | | | | |
| Loading EVR | 3 | 1 | 1 | 1 | 1 |
| [μg] | | | | | |
| ≥10 μm | 28.4 | 12.8 | 13.2 | 12.9 | 58.4 |
| ≥25 μm | 4.2 | 1.5 | 2.1 | 2.4 | 6.5 |
| ≥65 μm | 0.1 | 0.2 | 0.1 | 0.1 | 0.2 |
| ≥100 μm | 0 | 0 | 0 | 0 | 0 |

The crystal coating with trioctanoylglycerol/EVR during and after inflation shows a uniform coating with a uniform surface structure when inspected visually. The coating does not crumble off during inflation of the balloon.

In addition to excellent flexibility and virtually lossless adhesion, the crystal coating according to the invention also exhibits the required and necessary temperature stability, sterilizability (ETO sterilization is preferred) and shelf life.

2. Determination of the Loss of Active Agent or Coating During Implantation Using an In Vitro Model

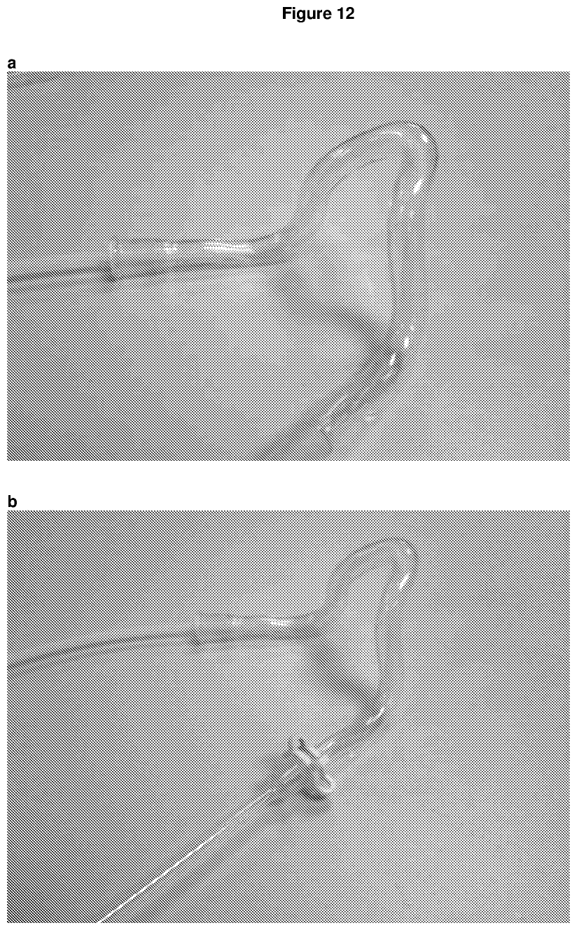

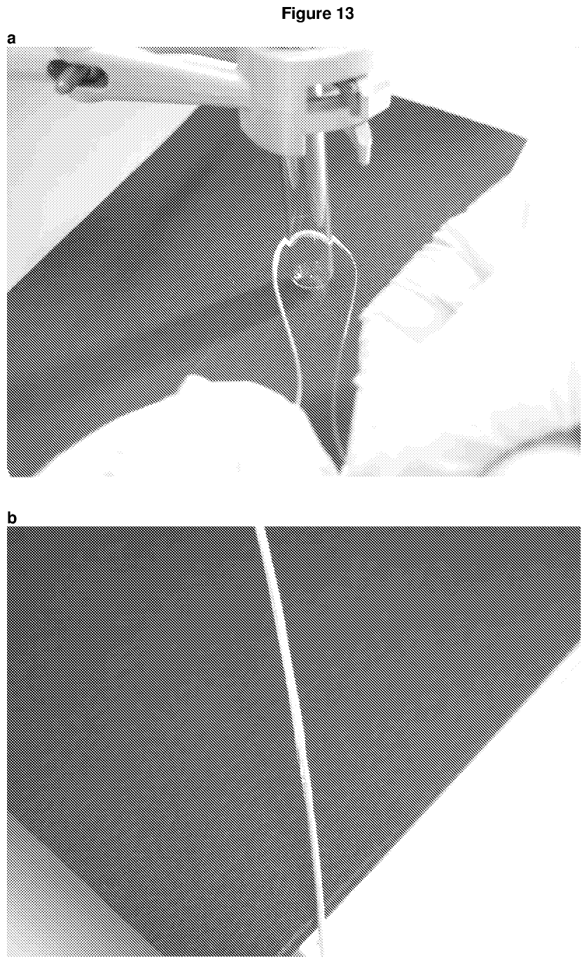

To simulate the natural often curved paths through the vessels that a balloon catheter must travel to the implantation site, a silicone tubing model is formed (see FIG. 12a and FIG. 12b) that mimics the natural course of the vessels in the organism.

The catheter is inserted into the silicone tube simulating the artery and inflated. The silicone tube was previously filled with a defined volume of pyrogen-free water. After 60 sec, the balloon is deflated (pull vacuum) and carefully pulled out. Care is taken to ensure that the liquid from the tube is completely collected in a container. Subsequently, it is rinsed with a defined amount of water and also collected. Particle analysis (particle size distribution and quantification) is performed via LPC (Liquid Particle Counter).

TABLE 16
Particle release of different coatings with everolimus crystal suspensions
containing 20% trioctanoylglycerol relative to everolimus during
inflation in PBS after in vitro determination by LPC.

| | Total particle count | | | |
|---|---|---|---|---|
| Sample | ≥10 μm | ≥25 μm | ≥65 μm | ≥100 μm |
| 6.0 × 40, 3 μg, 20% | 369 | 59 | 0 | 0 |
| Trioctanoylglycerol | | | | |
| 6.0 × 40, 1 μg, 20% | 24 | 25 | 0 | 0 |
| Trioctanoylglycerol | | | | |
| 5.0 × 40, 1 μg, 20% | 123 | 0 | 0 | 0 |
| Trioctanoylglycerol | | | | |
| 2.0 × 40, 1 μg, 20% | 89 | 26 | 0 | 0 |
| trioctanoylglycerol | | | | |

3. Determination of a Uniform Coating

For this purpose, the coated weighed balloon is fixed and inflated. The balloon is then cut with a scalpel into pieces of as equal size as possible, e.g. a 40 mm long balloon into 4 pieces, a 120 mm long balloon can be divided into 6 pieces. First, cut the balloon in half lengthwise and measure the layer thickness with a micrometer. Then the balloon is divided and the pieces are weighed and also the layer thicknesses are measured with the micrometer.

The coatings are each dissolved in a defined amount of acetone and the amount of active agent is determined via HPLC. The results are compared with each other, taking into account the balloon section areas.

In all cases, the presence of trioctanoylglycerol, or tridecanoylglycerol has been found to give a particularly stable and flexible coating, with the active agent also adhering very well in the form of crystals, which are only released during the contact time with the target site.

### Example 10

Comparison with Crystal Coatings not According to the Invention

Crystal Coatings According to WO 2015/039969 A1

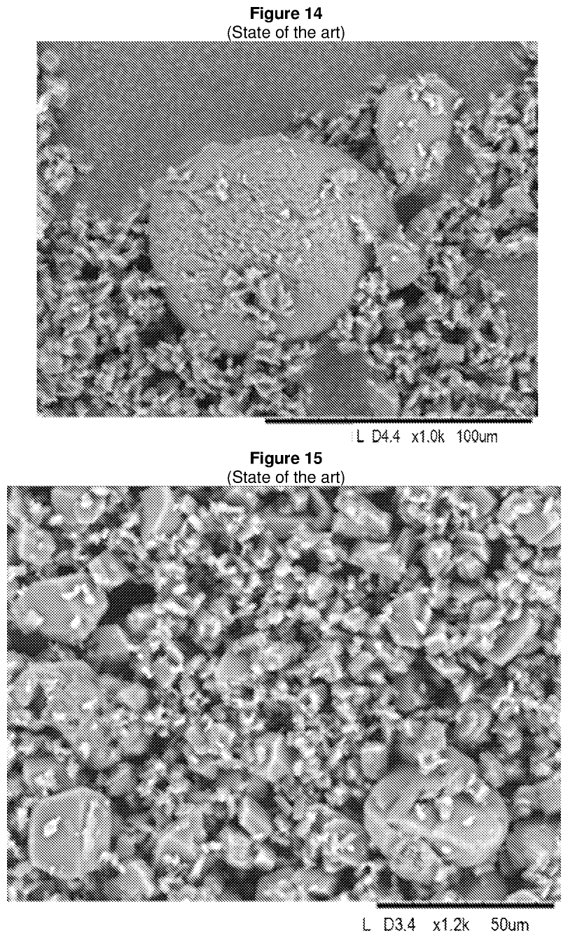

FIG. 14 shows a coating not according to the invention with rapamycin crystals according to WO 2015/039969 A1 at 1000× magnification. A too large almost round crystal surrounded by many quite small crystals of irregular shape and broad particle size distribution can be seen. FIG. 15 shows the non-inventive coating with rapamycin crystals according to WO 2015/039969 A1 at 1200× magnification. Numerous quite large crystals surrounded by many quite small crystals of irregular shape and broad particle size distribution can be seen.

Crystal Coatings of Rapamycin Microcrystals and “Solvent Bonding”

The extent to which limus-crystals can be applied as a dry substance (powder) to balloon catheters was tested. Among other things, the adhesion of the crystals was evaluated. Rapamycin crystals manufactured by Hemoteq were used for the experiments. PTA catheters with a balloon size of 4.0×60 mm were used for the application of the coating.

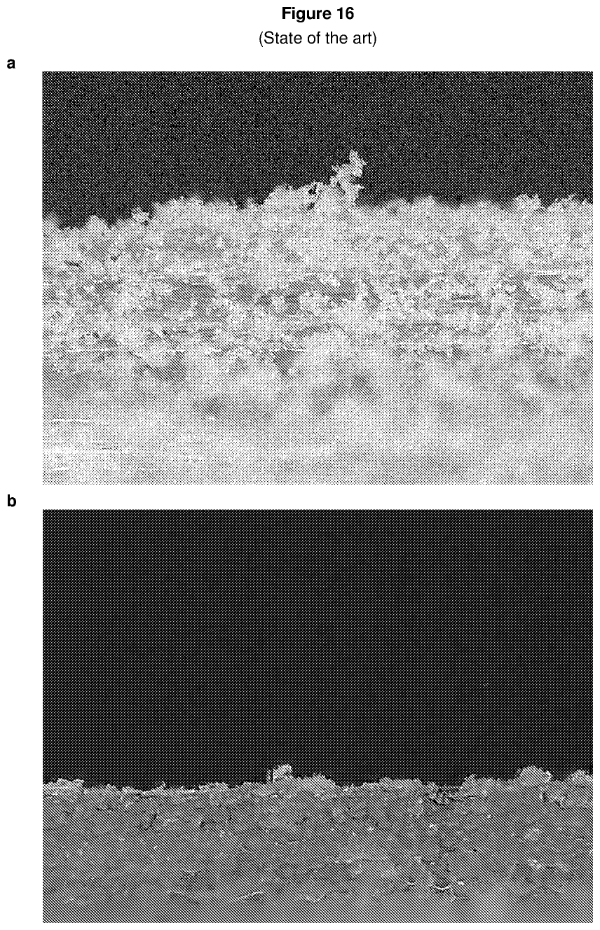

First, the application of the pure crystal powder was performed. For this purpose, the powder is filled into a custom-made bowl and brought into contact with the balloon. In the process, the rotating balloon picks up crystals that adhere to the surface. A solvent was then carefully sprayed on to slightly dissolve the crystals so that they would adhere better to the balloon surface after subsequent drying. In FIG. 16a), the coating with rapamycin crystals not according to the invention is shown at 200× magnification. FIG. 16b) clearly shows that the microcrystals of rapamycin do not remain intact and are dissolved. The “crumble tests” clearly showed that the total particle loss and particle release is very high. The coating adheres poorly to the balloon surface.

Crystal Coatings of Rapamycin Microcrystals with Base Coat of Commercial Adhesive or Trioctanoylglycerol Solution and Optional Topcoat with Trioctanoylglycerol Solution

The extent to which limus-crystals can be applied as a dry substance (powder) to balloon catheters was tested. Among other things, the adhesion of the crystals was evaluated. Sirolimus crystals manufactured by Hemoteq were used for the experiments. PTA catheters with a balloon size of 4.0×60 mm were used for the application of the coating.

First, a base coat was applied to ensure the adhesion of the crystals to the balloon. To test the suitability of the experimental setup, a commercially available medical adhesive (Henkel) was initially used.

Base coating of commercially available adhesive (Uhu): The adhesive is applied thinly directly from the tube and evenly distributed on the rotating balloon. This is followed by the application of the crystals.

Furthermore, trioctanoylglycerol was used as a base coating. The base coating is applied by pipetting on the rotating catheter. After approx. 10 min drying of the base coating, the pure crystal powder is applied. For this purpose, the powder is filled into a custom-made dish and brought into contact with the balloon. In the process, the rotating balloon picks up crystals that adhere to the surface.

Composition of the solution:

84.4% n-Heptane (volume %)

15.6% Ethyl acetate (volume %)

0.05% butylated hydroxytoluene (mass/volume)

200 μl of trioctanoylglycerol is dissolved in 2 ml of the solution. 2×50 μl of the base coating solution are applied.

When the trioctanoylglycerol was used, it could be shown that the crystals adhered to the balloon surface, but the adhesion of the crystals to each other was not sufficient.

In a third coating step, a topcoat with trioctanoylglycerol was therefore applied with a pipette to increase adhesion. The adhesion was evaluated by means of a “bend test”.

The bend test is a method in which the coated balloon is bent 2× around a glass tube of about 14 mm diameter. If many and/or larger fragments detach from the coating during this process, the adhesion is rated as insufficient.

Composition of the topcoat solution:

84.4% n-Heptane (volume %)

15.6% Ethyl acetate (volume %)

0.25% trioctanoylglycerol (mass/volume)

0.05% butylated hydroxytoluene (mass/volume)

2×30 μl top coat-solution was pipetted respectively.

When the topcoat solution was used, the crystals were found to adhere better to the balloon surface, and the adhesion of the crystals to each other was also improved, so that less particle release occurred in the edge impact test. However, the bending test and inflation of the balloon showed that the adhesion of the crystals to each other was not sufficient.

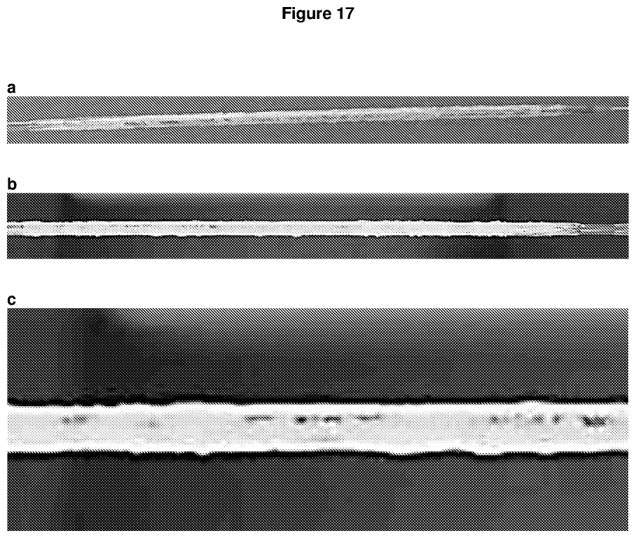

FIGS. 17 a-c show images of the balloon coating with topcoat. FIG. 17c is an enlargement of FIG. 17b. It is clearly visible to the naked eye that the coatings are not uniform and have larger areas where no microcrystals are present. In addition, the reproducibility is very poor with these coatings. Thus, particularly uniform, flexible and very well adherent coatings with microcrystals of rapamycin could not be produced in this way.

### Example 11

In Vivo Study with Sirolimus (SIR, Crystalline) and Everolimus (EVR, Crystalline) and Trioctanoylglycerol (GTC, 20 wt % for Active Agent) on PTA Catheters

The study was designed to determine the local pharmacokinetics of sirolimus and everolimus in the presence of trioctanoylglycerol. Three commercially available sirolimus and everolimus-eluting stents and balloon catheters were used for comparison. For comparison, a commercially available sirolimus-eluting balloon catheter (Magic Touch from Concept Medical) and a sirolimus-eluting stent (Orsiro from Biotronik) were included in the study. Because there is no commercially available everolimus-eluting balloon catheter, only an everolimus-eluting stent (Promus from Boston Scientific) could be included in the study as a comparison.

For this purpose, balloon catheters of different sizes were coated with EVR crystal suspension/GTC (3 μg/mm² EVR, 20 wt % regarding EVR) and SIR crystal suspension/GTC (3 μg/mm² SIR, 20 wt % regarding SIR). Thirty healthy domestic pigs (male, castrated) were available as experimental animals.

After implantation, the remaining drug residues are determined on the surfaces of both embodiments. It is clear that the transfer to the vessel wall worked very well and that only small residues remained on the balloon, which means that it can be assumed that the transfer to the vessel wall was extremely effective (see table 17).

TABLE 17
Average remaining drug content on the
PTA catheters after implantation.

| | Average active | |
|---|---|---|
| Samples | agent content* | SD |
| SCB-2 | | |
| 3.5-4.0 mm | 7.4% | 2.8% |
| 5.0-6.0 mm | 3.9% | 2.4% |
| ECB-4 | | |
| 3.5-4.0 mm | 3.0% | 1.0% |
| 5.0-6.0 mm | 17.1% | 5.6% |

*Averaged active agent residues on balloon after implantation in % in relation to nominal loading

The complementary active agent concentrations in the vessel walls after implantation and after 7 and 28 days also show successful drug delivery—also in comparison to the comparative sample. Thus, as a direct DCB comparison, the Magic Touch delivers much less sirolimus to the vessel wall than the SCB-2 according to the invention. After 7 days, the concentration of sirolimus in the SBC-2 is more comparable to the stent than to the Magic Touch, and this difference continues after 28 days. Thus, the SCB-2 according to the invention is definitely superior to the Magic Touch as DCB and to the DES Orsiro.

TABLE 18
For the sirolimus/trioctanoylglycerol group (SCB), follow-up values for
drug concentration in the artery after 1-2 h, 7 d and 28 d are as follows.

| | | | | Magic Touch DCB | | Sir-eluting stent | |
|---|---|---|---|---|---|---|---|
| Follow- | | SCB-2 | | (Reference) | | (reference) | |
| up | | Mean | SD | Mean | SD | Mean | SD |
| after | | [μg/g] | | [μg/g] | | [μg/g] | |
| 1-2 | h | 22.4 | 20.5 | 4.4 | 3.2 | — | — |
| 7 | d | 0.91 | 1.10 | 0.28 | 0.09 | 1.07 | 0.45 |
| 28 | d | 3.66 | 4.98 | 0.45 | 0.64 | 1.35 | 1.52 |

For the everolimus/trioctanoylglycerol balloon catheter (ECB), the delivery values are still significantly increased and better. The delivery of drug into the vessel wall is optimally increased. The comparison with the everolimus eluting stent shows the superiority of the balloon catheter according to the invention also over the stent, which even remains in the body until explanation.

TABLE 19
For the everolimus/trioctanoylglycerol group (ECB),
follow-up values for drug concentration in arteries
after 1-2 h, 7 d and 28 d are as follows.

| | |
|---|---|
| ECB | Promus EES* (reference) |

| | | | | |
|---|---|---|---|---|
| Follow-up | Mean | SD | Mean | SD |

| | | |
|---|---|---|
| after | [μg/g] | [μg/g] |

| | | | | | |
|---|---|---|---|---|---|
| 1-2 | h | 207.3 | 93.1 | n/a | n/a |
| 7 | d | 13.1 | 21.4 | 1.96 | 2.15 |
| 28 | d | 1.80 | 2.56 | 2.18 | 1.85 |

*EES: Everolimus eluting stent

In addition, it can be seen that the recovery rate of the active agent makes it clear that the active agents have actually arrived at their destination and in the vessel wall. (HPLC measurements). The small missing residue remained on the balloon catheter (see Table 20). These data are further evidence of the stability and flexibility as well as the very good availability of the active agents upon inflation at the target site.

TABLE 20
Recovery rate of the active agents sirolimus
(SCB) and everolimus (ECB) after 28 d (HPLC).

| | | | | | |
|---|---|---|---|---|---|
| | | Recovery rate | | | |
| | Nominal | active substance | SD | Purity | SD |
| Samples | load [μg] | (n = 5) [%] | [%] | (n = 5) [%] | [%] |

SCB

| | | | | | |
|---|---|---|---|---|---|
| 4.0 × 20 mm | 754.0 | 89.6 | 99.0 | 99.0 | 0.0 |
| 5.0 × 40 mm | 1885.0 | 79.0 | 97.6 | 97.6 | 0.2 |
| 6.0 × 40 mm | 2261.9 | 96.9 | 97.6 | 97.9 | 0.2 |

EC

| | | | | | |
|---|---|---|---|---|---|
| 3.5 × 20 mm | 659.7 | 114.6 | 99.5 | 99.5 | 0.1 |
| 4.0 × 20 mm | 754.0 | 106.3 | 99.5 | 99.5 | 0.0 |
| 5.0 × 40 mm | 1885.0 | 96.3 | 98.8 | 98.8 | 0.0 |
| 6.0 × 40 mm | 2261.9 | 91.5 | 98.9 | 98.9 | 0.2 |

What is claimed is:

1. A suspension for coating of a medical device selected from a catheter balloon, a balloon catheter, a stent, or a cannula, the suspension containing:
a) at least one tri-O-acylglycerol selected from the group consisting of trioctanoylglycerol, trinonanoylglycerol, tridecanoylglycerol, and triundecanoylglycerol,
b) at least one limus active agent in the form of microcrystals, and
c) a solvent or a solvent mixture in which the at least one tri-O-acylglycerol dissolves and in which the microcrystals of the at least one limus active agent do not dissolve.

2. The suspension according to claim 1, wherein the at least one limus active agent is selected from the group comprising or consisting of rapamycin, everolimus, zotarolimus, umirolimus, deforolimus, myolimus, novolimus, pimecrolimus, ridaforolimus, tacrolimus, and temsirolimus.

3. The suspension according to claim 1, wherein the at least one tri-O-acylglycerol and the limus active agent are present in a mass ratio of 10%-30% tri-O-acylglycerol to 90%-70% limus active agent.

4. The suspension according to claim 1, wherein the at least one limus active agent is in the form of microcrystals having a crystal size in the range of 1 μm to 300 μm.

5. The suspension according to claim 1, wherein at least 70% of the at least one limus active agent is in the form of microcrystals having a crystal size ranging from 10 μm to 50 μm.

6. The suspension according to claim 1, wherein the at least one limus active agent has a crystallinity of at least 90% by weight.

7. The suspension according to claim 1, wherein the solvent is a non-solvent having a dielectric constant εᵣ at 20° C. of 2.0 or the solvent mixture contains at least 50% by volume of a non-solvent having a dielectric constant εᵣ at 20° C. of 2.0.

8. The suspension according to claim 1, wherein the solvent mixture is a mixture of at least one polar organic solvent having an n-octanol-water partition coefficient log K_{OW} of −0.5 to +1.5 and a dielectric constant εᵣ at 20° C. of 5.0 to 30, and at least one nonpolar organic solvent having a dielectric constant εᵣ at 20° C. of ≤3.0 and an n-octanol-water partition coefficient log K_{OW} of ≥3.0.

9. A method of preparing the suspension according to claim 1 comprising the following steps:
a) dissolving at least one tri-O-acylglycerol selected from the group consisting of trioctanoylglycerol, trinonanoylglycerol, tridecanoylglycerol, and triundecanoylglycerol in a solvent or a solvent mixture;
b) preparing a suspension of at least one limus active agent in the form of microcrystals and the solution from step a),
wherein the microcrystals of the at least one limus active agent do not dissolve in the solution of step a).

10. The method according to claim 9, wherein the at least one limus active agent is selected from the group comprising or consisting of rapamycin, everolimus, zotarolimus, umirolimus, deforolimus, myolimus, novolimus, pimecrolimus, ridaforolimus, tacrolimus, and temsirolimus.

11. The method according to claim 9, wherein the at least one limus active agent is in the form of microcrystals having a crystal size in the range of 1 μm to 300 μm.

12. The method according to claim 9, wherein at least 70% of the at least one limus active agent is in the form of microcrystals having a crystal size in the range of 10 μm to 50 μm.

13. The method according to claim 9, wherein the at least one limus active agent has a crystallinity of at least 90% by weight.

14. The method according to claim 9, wherein the solvent is a non-solvent having a dielectric constant εᵣ at 20° C. of ≤2.0 or the solvent mixture contains at least 50% by volume of a non-solvent having a dielectric constant εᵣ at 20° C. of ≤2.0.

15. The method according to claim 9, wherein the solvent mixture is a mixture of at least one polar organic solvent having an n-octanol-water partition coefficient log K_{OW} of −0.5 to +1.5 and a dielectric constant εᵣ at 20° C. of 5.0 to 30, and at least one nonpolar organic solvent having a dielectric constant εᵣ at 20° C. of ≤3.0 and an n-octanol-water partition coefficient log K_{OW} of ≥3.0.

16. A method for coating of a medical device selected from a catheter balloon, a balloon catheter, a stent, or a cannula, comprising the following steps:
a) providing the medical device with a medical device surface,
b) providing a suspension containing at least one tri-O-acylglycerol selected from the group consisting of trioctanoylglycerol, trinonanoylglycerol, tridecanoylglycerol, and triundecanoylglycerol, at least one limus active agent in the form of microcrystals, and a solvent or a solvent mixture, in which the at least one tri-O-acylglycerol dissolves and in which the microcrystals of the at least one limus active agent do not dissolve, and
c) applying the coating suspension to the surface of the medical device by means of a syringe method, pipetting method, capillary method, fold spraying method, dipping method, spraying method, dragging method, thread dragging method, drop dragging method, or rolling method.

17. The method according to claim 16, further comprising the following step d) drying the coating.

18. (canceled)

19. A medical device selected from a catheter balloon, a balloon catheter, a stent, or a cannula, coated with the suspension according to claim 1 and subsequent drying of the coating.

20. A medical device selected from from a catheter balloon, a balloon catheter, a stent, or a cannula coated with at least one tri-O-acylglycerol selected from the group consisting of trioctanoylglycerol, trinonanoylglycerol, tridecanoylglycerol, and triundecanoylglycerol, and at least one limus active agent in the form of microcrystals.

21. The medical device of claim 20, wherein the limus active agent is selected from the group comprising or consisting of rapamycin, everolimus, biolimus A9, pimecrolimus, zotarolimus, tacrolimus, deforolimus, myolimus, novolimus, ridaforolimus, and temsirolimus.

22. The medical device of claim 20, wherein the at least one tri-O-acylglycerol and the at least one limus active agent are present in a mass ratio of 10%-30% tri-O-acylglycerol to 90%-70% limus active agent.

23. The medical device according to claim 20, wherein the at least one limus active agent is in the form of microcrystals having a crystal size in the range of 1 μm to 300 μm.

24. The medical device according to claim 20, wherein at least 70% of the at least one limus active agent is in the form of microcrystals having a crystal size in the range of 10 μm to 50 μm.

25. The medical device according to claim 20, wherein the at least one limus active agent has a crystallinity of at least 90% by weight.

26. The medical device according to claim 20, wherein a biostable or biodegradable, bioactive or bioinert polymeric, metallic or ceramic layer is present beneath the layer of the at least one tri-O-acylglycerol and the at least one limus active agent.
